SEQUENCE LISTING (198 sequences)

SEQ ID NO: 1 - protein, 117 aa, Artificial Sequence
  F4 Heavy Chain Variable Region
EVQLVESGGGLVKPGGSLKLSCAASGFTFSDYGMHWVRQAPEKGLEWVAFISSGSNTIYYADIVKGRFAISRDNAKNTLFLQMASLRSEDTALYYCARLAEWDVAYWGQGTLVTVSA

SEQ ID NO: 2 - protein, 108 aa, Artificial Sequence
  F4 Light Chain Variable Region
DIVLTQSPATLSVTPGDRISLSCRASQNINNNLHWYQQKSHESPRLLIKFASQSISGIPSRFSGSGSGTDFTLNINGVETEDFGMYFCQQIYSWPQLTFGAGTRLELK

SEQ ID NO: 3 - protein, 118 aa, Artificial Sequence
  F5 Heavy Chain Variable Region
EVQLVESGGELVKPGGSLKLSCAVSGFTFSNYGMSWVRQTPDKRLEWVATISSGGSYTYYPDSVKGRFTISRDNDKNTLYLQMSSLKSEDTAMYYCSTQGSSGYVGYWGQGTTLTVSS

SEQ ID NO: 4 - protein, 106 aa, Artificial Sequence
  F5 Light Chain Variable Region
DIQMTQSPSSLSAFLGGKVTITCKASQDITNFIGWYQHKPGKGPRLLISYTSILESGIPSRFSGSGSGRDYSFSISNLEPEDIATYYCLQYYNLWTFGGGTKLEIK

SEQ ID NO: 5 - protein, 118 aa, Artificial Sequence
  F7 Heavy Chain Variable Region
EFQLQQSGPELVKPGASVKISCKASGYSFTDYNMNWVKQSNGKSLEWIGVIDPNYGTTNYNQKFVGKATLTVDQSSITAYMQLNSLTAEDSAVYFCAIKGYGNPAAYWGQGTLVTVSA

SEQ ID NO: 6 - protein, 106 aa, Artificial Sequence
  F7 Light Chain Variable Region
DIQMTQSPSSLSASLGGKVTITCKASQDINKYLAWYQHEPGKGPRLLIRYTSILESGIPSRFSGSGSGRDYSFSISNLEPEDIATYYCLQYYNLWTFGGGTKLEIK

SEQ ID NO: 7 - protein, 121 aa, Artificial Sequence
  F8 Heavy Chain Variable Region
EVMLVESGGGLVKPGGSLKLSCVASGFTLSTYAMSWVRQTPEKRLEWVATISGGGGDTYHLDTVKGRFTISRDNAKNTLYLQMSSLRSEDTALYYCARQSHYGSSYYFDNWGQGTTLTVSS

SEQ ID NO: 8 - protein, 107 aa, Artificial Sequence
  F8 Light Chain Variable Region
DIQMTQSPASLSASVGEIVTIICRVSENIDSYLAWYQQKQGKSPQLLVYAATNLADGVPSRFSGSGSGSQYSLKINSLQSEDVARYYCQHYYTTPPTFGGGTKLDIK

SEQ ID NO: 9 - protein, 120 aa, Artificial Sequence
  F10 Heavy Chain Variable Region
QVQLQQSGPELVKPGASVKISCKASGYAFSSSWMNWVRQRPGKGLEWIGRIYPGDGYTHYNGMFKGKATLTADKSSSTGYMQLSSLTSEDSAVYFCTRHGDFPYWYFDVWGTGTTVTVSS

SEQ ID NO: 10 - protein, 107 aa, Artificial Sequence
  F10 Light Chain Variable Region
DIQMTQSPASLSASVGESVTITCRASENIDSYLAWYQQKQGKSPQLLVYAATNLAVGVPSRFSGSGSGTQYTLKINSLQSEDVARYYCQHHYSTPPTFGGGTKLEIK

SEQ ID NO: 11 - protein, 117 aa, Artificial Sequence
  F17 Heavy Chain Variable Region
DVQLVESGGGLVQPGGSRKLSCAASGFTFSDFGMHWIRQAPERGLEWVAYMSYTPGTFHYADTVKDRFFISRDNAKNTLFLQMTSLRSDDTAMYYCARVHVGTVDYWGQGTSVTVSS

SEQ ID NO: 12 - protein, 108 aa, Artificial Sequence
  F17 Light Chain Variable Region
DVVLTQSPATLSVTPGDSVSLSCRASQNINNNLHWFQQKSHESPRLLIKYASQSISGIPSRFSGSGSGTDFTLSINNVETEDFGIYFCQQSNSWPALTFGTGTKVELK

SEQ ID NO: 13 - protein, 121 aa, Artificial Sequence
  F19 Heavy Chain Variable Region
EVKLDETGGGLVQPGRPMKLSCVASGFTFSDYWMNWVRQSPDKGLEWVAQIGNKFHNYETYYSDSVKGRFTISRDDSKSSVYLQMNSLRVEDTGIYYCTKLGRGYYVMDYWGQGTSVTVSS

SEQ ID NO: 14 - protein, 107 aa, Artificial Sequence
  F19 Light Chain Variable Region
DIQMTQTTSSLSASLGDRVTLNCRASQDITNHLNWFQQKPDGTFQLLIYYTSRLHSGVPSRFSGSGSGTDYSLTISNLEQEDFATYFCQQDSQHPWTFGGGTKLEIK

SEQ ID NO: 15 - protein, 121 aa, Artificial Sequence
  F20 Heavy Chain Variable Region
QVQLQQSGAELVKPGASVQLSCKASGYTFASYYLYWVKQRPGQGLEWIGEINPRSGGTNFNEKFKSKATVTVDKSSSTAYMQLSSLTSEDSAVYYCSRSGRLRGFYTMDYWGQGTSVTVSS

SEQ ID NO: 16 - protein, 106 aa, Artificial Sequence
  F20 Light Chain Variable Region
NIVMTQSPKSMSVSVGERVTLSCKAGENVGSYVSWYQQKPEQSPELLIYGASNRYTGVPDRFTGSGSATDFTLTISSVQAEDLADYYCGQTYRFLTFGAGTKLELK

SEQ ID NO: 17 - protein, 8 aa, Artificial Sequence
  F4 HCDR1
GFTFSDYG

SEQ ID NO: 18 - protein, 8 aa, Artificial Sequence
  F4 HCDR2
ISSGSNTI

SEQ ID NO: 19 - protein, 10 aa, Artificial Sequence
  F4 HCDR3
ARLAEWDVAY

SEQ ID NO: 20 - protein, 8 aa, Artificial Sequence
  F5 HCDR1
GFTFSNYG

SEQ ID NO: 21 - protein, 8 aa, Artificial Sequence
  F5 HCDR2
ISSGGSYT

SEQ ID NO: 22 - protein, 11 aa, Artificial Sequence
  F5 HCDR3
STQGSSGYVGY

SEQ ID NO: 23 - protein, 8 aa, Artificial Sequence
  F7 HCDR1
GYSFTDYN

SEQ ID NO: 24 - protein, 8 aa, Artificial Sequence
  F7 HCDR2
IDPNYGTT

SEQ ID NO: 25 - protein, 11 aa, Artificial Sequence
  F7 HCDR3
AIKGYGNPAAY

SEQ ID NO: 26 - protein, 8 aa, Artificial Sequence
  F8 HCDR1
GFTLSTYA

SEQ ID NO: 27 - protein, 8 aa, Artificial Sequence
  F8 HCDR2
ISGGGGDT

SEQ ID NO: 28 - protein, 14 aa, Artificial Sequence
  F8 HCDR3
ARQSHYGSSYYFDN

SEQ ID NO: 29 - protein, 8 aa, Artificial Sequence
  F10 HCDR1
GYAFSSSW

SEQ ID NO: 30 - protein, 8 aa, Artificial Sequence
  F10 HCDR2
IYPGDGYT

SEQ ID NO: 31 - protein, 13 aa, Artificial Sequence
  F10 HCDR3
TRHGDFPYWYFDV

SEQ ID NO: 32 - protein, 8 aa, Artificial Sequence
  F17 HCDR1
GFTFSDFG

SEQ ID NO: 33 - protein, 8 aa, Artificial Sequence
  F17 HCDR2
MSYTPGTF

SEQ ID NO: 34 - protein, 10 aa, Artificial Sequence
  F17 HCDR3
ARVHVGTVDY

SEQ ID NO: 35 - protein, 8 aa, Artificial Sequence
  F19 HCDR1
GFTFSDYW

SEQ ID NO: 36 - protein, 10 aa, Artificial Sequence
  F19 HCDR2
IGNKFHNYET

SEQ ID NO: 37 - protein, 12 aa, Artificial Sequence
  F19 HCDR3
TKLGRGYYVMDY

SEQ ID NO: 38 - protein, 8 aa, Artificial Sequence
  F20 HCDR1
GYTFASYY

SEQ ID NO: 39 - protein, 8 aa, Artificial Sequence
  F20 HCDR2
INPRSGGT

SEQ ID NO: 40 - protein, 14 aa, Artificial Sequence
  F20 HCDR3
SRSGRLRGFYTMDY

SEQ ID NO: 41 - protein, 6 aa, Artificial Sequence
  F4 LCDR1
QNINNN

SEQ ID NO: 42 - protein, 3 aa, Artificial Sequence
  F4 LCDR2
FAS

SEQ ID NO: 43 - protein, 10 aa, Artificial Sequence
  F4 LCDR3
QQIYSWPQLT

SEQ ID NO: 44 - protein, 6 aa, Artificial Sequence
  F5 LCDR1
QDITNF

SEQ ID NO: 45 - protein, 3 aa, Artificial Sequence
  F5 LCDR2
YTS

SEQ ID NO: 46 - protein, 8 aa, Artificial Sequence
  F5 LCDR3
LQYYNLWT

SEQ ID NO: 47 - protein, 6 aa, Artificial Sequence
  F7 LCDR1
QDINKY

SEQ ID NO: 48 - protein, 3 aa, Artificial Sequence
  F7 LCDR2
YTS

SEQ ID NO: 49 - protein, 8 aa, Artificial Sequence
  F7 LCDR3
LQYYNLWT

SEQ ID NO: 50 - protein, 6 aa, Artificial Sequence
  F8 LCDR1
ENIDSY

SEQ ID NO: 51 - protein, 3 aa, Artificial Sequence
  F8 LCDR2
AAT

SEQ ID NO: 52 - protein, 9 aa, Artificial Sequence
  F8 LCDR3
QHYYTTPPT

SEQ ID NO: 53 - protein, 6 aa, Artificial Sequence
  F10 LCDR1
ENIDSY

SEQ ID NO: 54 - protein, 3 aa, Artificial Sequence
  F10 LCDR2
AAT

SEQ ID NO: 55 - protein, 9 aa, Artificial Sequence
  F10 LCDR3
QHHYSTPPT

SEQ ID NO: 56 - protein, 6 aa, Artificial Sequence
  F17 LCDR1
QNINNN

SEQ ID NO: 57 - protein, 3 aa, Artificial Sequence
  F17 LCDR2
YAS

SEQ ID NO: 58 - protein, 10 aa, Artificial Sequence
  F17 LCDR3
QQSNSWPALT

SEQ ID NO: 59 - protein, 6 aa, Artificial Sequence
  F19 LCDR1
QDITNH

SEQ ID NO: 60 - protein, 3 aa, Artificial Sequence
  F19 LCDR2
YTS

SEQ ID NO: 61 - protein, 9 aa, Artificial Sequence
  F19 LCDR3
QQDSQHPWT

SEQ ID NO: 62 - protein, 6 aa, Artificial Sequence
  F20 LCDR1
ENVGSY

SEQ ID NO: 63 - protein, 3 aa, Artificial Sequence
  F20 LCDR2
GAS

SEQ ID NO: 64 - protein, 8 aa, Artificial Sequence
  F20 LCDR3
GQTYRFLT

SEQ ID NO: 65 - protein, 10 aa, Artificial Sequence
  F4 HCDR1
GFTFSDYGMH

SEQ ID NO: 66 - protein, 17 aa, Artificial Sequence
  F4 HCDR2
FISSGSNTIYYADIVKG

SEQ ID NO: 67 - protein, 10 aa, Artificial Sequence
  F4 HCDR3
ARLAEWDVAY

SEQ ID NO: 68 - protein, 10 aa, Artificial Sequence
  F5 HCDR1
GFTFSNYGMS

SEQ ID NO: 69 - protein, 17 aa, Artificial Sequence
  F5 HCDR2
TISSGGSYTYYPDSVKG

SEQ ID NO: 70 - protein, 11 aa, Artificial Sequence
  F5 HCDR3
STQGSSGYVGY

SEQ ID NO: 71 - protein, 10 aa, Artificial Sequence
  F7 HCDR1
GYSFTDYNMN

SEQ ID NO: 72 - protein, 17 aa, Artificial Sequence
  F7 HCDR2
VIDPNYGTTNYNQKFVG

SEQ ID NO: 73 - protein, 11 aa, Artificial Sequence
  F7 HCDR3
AIKGYGNPAAY

SEQ ID NO: 74 - protein, 10 aa, Artificial Sequence
  F8 HCDR1
GFTLSTYAMS

SEQ ID NO: 75 - protein, 17 aa, Artificial Sequence
  F8 HCDR2
TISGGGGDTYHLDTVKG

SEQ ID NO: 76 - protein, 14 aa, Artificial Sequence
  F8 HCDR3
ARQSHYGSSYYFDN

SEQ ID NO: 77 - protein, 10 aa, Artificial Sequence
  F10 HCDR1
GYAFSSSWMN

SEQ ID NO: 78 - protein, 17 aa, Artificial Sequence
  F10 HCDR2
RIYPGDGYTHYNGMFKG

SEQ ID NO: 79 - protein, 13 aa, Artificial Sequence
  F10 HCDR3
TRHGDFPYWYFDV

SEQ ID NO: 80 - protein, 10 aa, Artificial Sequence
  F17 HCDR1
GFTFSDFGMH

SEQ ID NO: 81 - protein, 17 aa, Artificial Sequence
  F17 HCDR2
YMSYTPGTFHYADTVKD

SEQ ID NO: 82 - protein, 10 aa, Artificial Sequence
  F17 HCDR3
ARVHVGTVDY

SEQ ID NO: 83 - protein, 10 aa, Artificial Sequence
  F19 HCDR1
GFTFSDYWMN

SEQ ID NO: 84 - protein, 19 aa, Artificial Sequence
  F19 HCDR2
QIGNKFHNYETYYSDSVKG

SEQ ID NO: 85 - protein, 12 aa, Artificial Sequence
  F19 HCDR3
TKLGRGYYVMDY

SEQ ID NO: 86 - protein, 10 aa, Artificial Sequence
  F20 HCDR1
GYTFASYYLY

SEQ ID NO: 87 - protein, 17 aa, Artificial Sequence
  F20 HCDR2
EINPRSGGTNFNEKFKS

SEQ ID NO: 88 - protein, 14 aa, Artificial Sequence
  F20 HCDR3
SRSGRLRGFYTMDY

SEQ ID NO: 89 - protein, 11 aa, Artificial Sequence
  F4 LCDR1
RASQNINNNLH

SEQ ID NO: 90 - protein, 7 aa, Artificial Sequence
  F4 LCDR2
FASQSIS

SEQ ID NO: 91 - protein, 10 aa, Artificial Sequence
  F4 LCDR3
QQIYSWPQLT

SEQ ID NO: 92 - protein, 11 aa, Artificial Sequence
  F5 LCDR1
KASQDITNFIG

SEQ ID NO: 93 - protein, 7 aa, Artificial Sequence
  F5 LCDR2
YTSILES

SEQ ID NO: 94 - protein, 8 aa, Artificial Sequence
  F5 LCDR3
LQYYNLWT

SEQ ID NO: 95 - protein, 11 aa, Artificial Sequence
  F7 LCDR1
KASQDINKYLA

SEQ ID NO: 96 - protein, 7 aa, Artificial Sequence
  F7 LCDR2
YTSILES

SEQ ID NO: 97 - protein, 8 aa, Artificial Sequence
  F7 LCDR3
LQYYNLWT

SEQ ID NO: 98 - protein, 11 aa, Artificial Sequence
  F8 LCDR1
RVSENIDSYLA

SEQ ID NO: 99 - protein, 7 aa, Artificial Sequence
  F8 LCDR2
AATNLAD

SEQ ID NO: 100 - protein, 9 aa, Artificial Sequence
  F8 LCDR3
QHYYTTPPT

SEQ ID NO: 101 - protein, 11 aa, Artificial Sequence
  F10 LCDR1
RASENIDSYLA

SEQ ID NO: 102 - protein, 7 aa, Artificial Sequence
  F10 LCDR2
AATNLAV

SEQ ID NO: 103 - protein, 9 aa, Artificial Sequence
  F10 LCDR3
QHHYSTPPT

SEQ ID NO: 104 - protein, 11 aa, Artificial Sequence
  F17 LCDR1
RASQNINNNLH

SEQ ID NO: 105 - protein, 7 aa, Artificial Sequence
  F17 LCDR2
YASQSIS

SEQ ID NO: 106 - protein, 10 aa, Artificial Sequence
  F17 LCDR3
QQSNSWPALT

SEQ ID NO: 107 - protein, 11 aa, Artificial Sequence
  F19 LCDR1
RASQDITNHLN

SEQ ID NO: 108 - protein, 7 aa, Artificial Sequence
  F19 LCDR2
YTSRLHS

SEQ ID NO: 109 - protein, 9 aa, Artificial Sequence
  F19 LCDR3
QQDSQHPWT

SEQ ID NO: 110 - protein, 11 aa, Artificial Sequence
  F20 LCDR1
KAGENVGSYVS

SEQ ID NO: 111 - protein, 7 aa, Artificial Sequence
  F20 LCDR2
GASNRYT

SEQ ID NO: 112 - protein, 8 aa, Artificial Sequence
  F20 LCDR3
GQTYRFLT

SEQ ID NO: 113 - protein, 118 aa, Artificial Sequence
  F5-H1 Heavy Chain Variable Region
EVQLLESGGGLVQPGGSLRLSCAVSGFTFSNYGMSWVRQAPGKGLEWVATISSGGSYTYYPDSVKGRFTISRDNSKNTLYLQMNSLRAEDTAVYYCSTQGSSGYVGYWGQGTLVTVSS

SEQ ID NO: 114 - protein, 118 aa, Artificial Sequence
  F5-H2 Heavy Chain Variable Region
EVQLVESGGGLVQPGGSLRLSCAVSGFTFSNYGMSWVRQAPGKGLEWVATISSGGSYTYYPDSVKGRFTISRDNSKNTLYLQMNSLRAEDTAVYYCSTQGSSGYVGYWGQGTLVTVSS

SEQ ID NO: 115 - protein, 118 aa, Artificial Sequence
  F5-H3 Heavy Chain Variable Region
EVQLVESGGGLVQPGGSLRLSCAVSGFTFSNYGMSWVRQAPGKGLEWVATISSGGSYTYYPDSVKGRFTISRDNSKNTLYLQMSSLRAEDTAVYYCSTQGSSGYVGYWGQGTLVTVSS

SEQ ID NO: 116 - protein, 118 aa, Artificial Sequence
  F5-H4 Heavy Chain Variable Region
EVQLVESGGGLVQPGGSLRLSCAVSGFTFSNYGMSWVRQAPGKGLEWVATISSGGSYTYYPDSVKGRFTISRDNDKNTLYLQMSSLRAEDTAVYYCSTQGSSGYVGYWGQGTLVTVSS

SEQ ID NO: 117 - protein, 106 aa, Artificial Sequence
  F5-L1 Light Chain Variable Region
DIQMTQSPSSVSASVGDRVTITCKASQDITNFIGWYQHKPGKAPKLLISYTSILESGVPSRFSGSGSGRDYTLTISSLQPEDFATYYCLQYYNLWTFGGGTKVEIK

SEQ ID NO: 118 - protein, 106 aa, Artificial Sequence
  F5-L2 Light Chain Variable Region
DIQMTQSPSSVSASVGDRVTITCKASQDITNFIGWYQHKPGKAPKLLISYTSILESGVPSRFSGSGSGTDYTLTISSLQPEDFATYYCLQYYNLWTFGGGTKVEIK

SEQ ID NO: 119 - protein, 120 aa, Artificial Sequence
  F10-H1 Heavy Chain Variable Region
EVQLVQSGAEVKKPGSSVKVSCKASGYAFSSSWMNWVRQAPGQGLEWIGRIYPGDGYTHYNGMFKGRASLTADKSTSTGYMELSSLRSEDTAVFFCTRHGDFPYWYFDVWGRGTLVTVSP

SEQ ID NO: 120 - protein, 120 aa, Artificial Sequence
  F10-H2 Heavy Chain Variable Region
EVQLVQSGAEVKKPGSSVKVSCKASGYAFSSSWMNWVRQAPGQGLEWIGRIYPGDGYTHYNGMFKGRASLTADKSTSTGYMELSSLRSEDTAVFFCTRHGDFPYWYFDVWGRGTLVTVSS

SEQ ID NO: 121 - protein, 107 aa, Artificial Sequence
  F10-L1 Light Chain Variable Region
DIQMTQSPSTLSASVGDRVTITCRASENIDSYLAWYQQKPGRAPKLLVYAATNLAVGVPSRFSGSGSGTEYTLTISSLQSDDFATYYCQHHYSTPPTFGQGTKLEIK

SEQ ID NO: 122 - protein, 107 aa, Artificial Sequence
  F10-L2 Light Chain Variable Region
DIQMTQSPSTLSASVGDRVTITCRASENIDSYLAWYQQKPGRAPKLLVYAATNLAVGVPSRFSGSGSGTEYTLTISSLQPDDFATYYCQHHYSTPPTFGQGTKLEIK

SEQ ID NO: 123 - protein, 107 aa, Artificial Sequence
  F10-L3 Light Chain Variable Region
DIQMTQSPSTLSASVGDRVTITCRASENIDSYLAWYQQKPGRAPKLLVYAATNLAVGVPSRFSGSGSGTEYTLTISSLQSEDFATYYCQHHYSTPPTFGQGTKLEIK

SEQ ID NO: 124 - protein, 117 aa, Artificial Sequence
  F17-H1 Heavy Chain Variable Region
EVQLVETGGGLIQPGGSLRLSCAASGFTFSDFGMHWIRQAPGKGLEWVAYMSYTPGTFHYADTVKDRFTISRDNSKNTLYLQMNSLRAEDTAVYYCARVHVGTVDYWGQGTLVTVSS

SEQ ID NO: 125 - protein, 117 aa, Artificial Sequence
  F17-H2 Heavy Chain Variable Region
QVQLVESGGGVVQPGRSLRLSCAASGFTFSDFGMHWIRQAPGKGLEWVAYMSYTPGTFHYADTVKDRFTISRDNSKNTLYLQMNSLRAEDTAVYYCARVHVGTVDYWGQGTLVTVSS

SEQ ID NO: 126 - protein, 108 aa, Artificial Sequence
  F17-L1 Light Chain Variable Region
EVVLTQSPATLSLSPGERATLSCRASQNINNNLHWFQQKPGQAPRLLIKYASQSISGIPARFSGSGSGTDFTLTISSLEPEDFAVYFCQQSNSWPALTFGQGTKVEIK

SEQ ID NO: 127 - protein, 108 aa, Artificial Sequence
  F17-L2 Light Chain Variable Region
EVVLTQSPATLSLSPGERATLSCRASQNINNNLHWFQQKPGQAPRLLIKYASQSISGIPARFSGSGSGTDFTLTISSLETEDFAVYFCQQSNSWPALTFGQGTKVEIK

SEQ ID NO: 128 - protein, 121 aa, Artificial Sequence
  F20-H1 Heavy Chain Variable Region
QVQLVQSGAEVKKPGSSVKVSCKASGYTFASYYLYWVRQAPGQGLEWIGEINPRSGGTNFNEKFKSRATVTVDKSTSTAYMELSSLRSEDTAVYYCSRSGRLRGFYTMDYWGQGTLVTVSS

SEQ ID NO: 129 - protein, 121 aa, Artificial Sequence
  F20-H2 Heavy Chain Variable Region
QVQLVQSGAEVKKPGSSVKVSCKASGYTFASYYLYWVRQAPGQGLEWIGEINPRSGGTNFNEKFKSRATVTVDASTSTAYMELSSLRSEDTAVYYCSRSGRLRGFYTMDYWGQGTLVTVSS

SEQ ID NO: 130 - protein, 121 aa, Artificial Sequence
  F20-H3 Heavy Chain Variable Region
QVQLVQSGAEVKKPGSSVKVSCKASGYTFASYYLYWVRQAPGQGLEWIGEINPRSGGTNFNEKFKSKATVTVDKSTNTAYMELSSLRSEDTAVYYCSRSGRLRGFYTMDYWGQGTLVTVSS

SEQ ID NO: 131 - protein, 106 aa, Artificial Sequence
  F20-L1 Light Chain Variable Region
DIVMTQSPDSLAVSLGERATINCKAGENVGSYVSWYQQKPGQPPKLLIYGASNRYTGVPDRFSGSGSATDFTLTISSLQAEDVAVYYCGQTYRFLTFGQGTKVEIK

SEQ ID NO: 132 - protein, 106 aa, Artificial Sequence
  F20-L2 Light Chain Variable Region
DIVMTQSPDSLAVSLGERATINCKAGENVGSYVSWYQQKPGQSPKLLIYGASNRYTGVPDRFSGSGSATDFTLTISSLQAEDVAVYYCGQTYRFLTFGQGTKVEIK

SEQ ID NO: 133 - protein, 106 aa, Artificial Sequence
  F20-L3 Light Chain Variable Region
DIQMTQSPSTLSASVGDRVTITCKAGENVGSYVSWYQQKPGKAPKLLIYGASNRYTGVPARFSGSGSATEFTLTISSLQPDDFATYYCGQTYRFLTFGQGTKVEVK

SEQ ID NO: 134 - protein, 106 aa, Artificial Sequence
  F20-L4 Light Chain Variable Region
DIQMTQSPSTLSASVGDRVTITCKAGENVGSYVSWYQQKPGKAPKLLIYGASNRYTGVPARFSGSGSATEFTLTISSLQPEDFATYYCGQTYRFLTFGQGTKVEVK

SEQ ID NO: 135 - protein, 257 aa, Homo sapiens
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 136 - protein, 117 aa, Artificial Sequence
  F4-H1 Heavy Chain Variable Region
QVQLVQSGAEVRKPGASVKVSCKASGFTFSDYGMHWVRQAPGQGLEWVAFISSGSNTIYYADIVKGRFTITRNNSTSTLYMELSSLRSEDTAIYYCARLAEWDVAYWGAGTLVTVSS

SEQ ID NO: 137 - protein, 117 aa, Artificial Sequence
  F4-H2 Heavy Chain Variable Region
QVQLVQSGAEVKKPGASVKVSCKASGFTFSDYGMHWVRQAPGQGLEWVAFISSGSNTIYYADIVKGRVTITRNNSTSTLYMELSSLRSEDTAIYYCARLAEWDVAYWGAGTLVTVSS

SEQ ID NO: 138 - protein, 117 aa, Artificial Sequence
  F4-H3 Heavy Chain Variable Region
EVQLVESGGGLVQPGRSLRLSCAASGFTFSDYGMHWVRQAPGKGLEWVAGISSGSNTIGYADSVQGRFTISRDNGKNSLYLQMNSLRAEDTALYYCARLAEWDVAYWGQGTMVTVSS

SEQ ID NO: 139 - protein, 108 aa, Artificial Sequence
  F4-L1 Light Chain Variable Region
DIQLTQSPSTLSASIGDRVTITCRASQNINNNLHWYQQKPGKAPKLLIKFASQSISGAPSRFSGSGSGTDFTLTISSLQPDDFATYFCQQIYSWPQLTFGGGTKLEIK

SEQ ID NO: 140 - protein, 108 aa, Artificial Sequence
  F4-L2 Light Chain Variable Region
DIQLTQSPSSLSASVGDRVTITCRASQNINNNLHWYQQKPGKAPKLLIKFASQSISGVPSRFSGSGSGTDFTLTISSLQPDDFATYFCQQIYSWPQLTFGGGTKLEIK

SEQ ID NO: 141 - protein, 108 aa, Artificial Sequence
  F4-L3 Light Chain Variable Region
EIVLTQSPGTLSLSPGERATLSCRASQNINNNLHWYQQKPGQAPRLLIKFASTRATGIPARFSGSGSGTDFTLTISRLEPEDLAVYFCQQIYSWPQLTFGQGTKVEIK

SEQ ID NO: 142 - protein, 118 aa, Artificial Sequence
  F7-H1 Heavy Chain Variable Region
QFQLVQSGAEVKKPGASVKVSCKASGYSFTDYNMHWVRQAPGQGLEWIGWIDPNYGTTNYAQKFQGRATLTVDQSISTAYMELSRLRSDDTAVYFCAIKGYGNPAAYWGQGTLVTVSS

SEQ ID NO: 143 - protein, 118 aa, Artificial Sequence
  F7-H2 Heavy Chain Variable Region
EFQLVESGAEVKKPGSSVKVSCKASGYSFTDYNFTWVRQAPGQGLEWIGRIDPNYGTTHYAPHLQGRATLTVDQSTSTAYLELRNLRSDDTAVYFCAIKGYGNPAAYWGQGTLVTVSS

SEQ ID NO: 144 - protein, 118 aa, Artificial Sequence
  F7-H3 Heavy Chain Variable Region
QVQLVQSGAEVKKPGASVKVSCKASGYSFTDYNFTWVRQAPGQGLEWIGRIDPNYGTTHYAPHLQGRATLTVDQSTSTAYLELRSLRSDDTAVYFCAIKGYGNPAAYWGQGTLVTVSS

SEQ ID NO: 145 - protein, 106 aa, Artificial Sequence
  F7-L1 Light Chain Variable Region
DIVMTQSPDSLAVSLGERATINCKASQDINKYLAWYQHKPGQPPKLLIRYTSTRESGVPDRFSGSGSGRDYTLTISSLQAEDVAVYYCLQYYNLWTFGGGTKVEIK

SEQ ID NO: 146 - protein, 106 aa, Artificial Sequence
  F7-L2 Light Chain Variable Region
DIVMTQSPATLSVSPGERATLSCRASQDINKYLAWYQHKPGQAPRLLIRYTSTRATGVPARFSGSGSGREYTLTISSLQSEDFAVYYCLQYYNLWTFGQGTRLEIK

SEQ ID NO: 147 - protein, 106 aa, Artificial Sequence
  F7-L3 Light Chain Variable Region
DIVMTQSPATLSVSPGERATLSCRASQDINKYLAWYQHKPGQAPRLLIRYTSTRATGIPARFSGSGSGTEFTLTISSLQSEDFAVYYCLQYYNLWTFGQGTRLEIK

SEQ ID NO: 148 - protein, 121 aa, Artificial Sequence
  F8-H1 Heavy Chain Variable Region
LVNLVESGGGVVQPGRSLRLSCAASGFTLSTYAMHWVRQAPGKGLEWVAVISGGGGDTYYADSVKGRFTISRDNSKNTLYLQMNSLRAEDTAVFYCARQSHYGSSYYFDNWGQGTLVTVSS

SEQ ID NO: 149 - protein, 121 aa, Artificial Sequence
  F8-H2 Heavy Chain Variable Region
QVQLVESGGGVVQPGRSLRLSCAASGFTLSTYAMNWVRQAPAKGLEWVAIISGGGGDTYYADSVKGRFTISRDNSKNTLYLQMNGLRAEDTAVYYCARQSHYGSSYYFDNWGQGTLVTVSS

SEQ ID NO: 150 - protein, 121 aa, Artificial Sequence
  F8-H3 Heavy Chain Variable Region
QVQLVESGGGVVQPGRSLRLSCAASGFTLSTYAMSWVRQAPAKGLEWVAIISGGGGDTYYADSVKGRFTISRDNSKNTLYLQMNGLRAEDTAVYYCARQSHYGSSYYFDNWGQGTLVTVSS

SEQ ID NO: 151 - protein, 107 aa, Artificial Sequence
  F8-L1 Light Chain Variable Region
EIQMTQSPATLSLSPGERATLSCRVSENIDSYLAWYQQKPGQAPRLLVYAATNRATGIPARFSGSGSGSDYTLTISSLEPEDFAVYYCQHYYTTPPTFGQGTKVEIK

SEQ ID NO: 152 - protein, 107 aa, Artificial Sequence
  F8-L2 Light Chain Variable Region
EIVMTQSPDFQSVTPKEKVTITCRVSENIDSYLAWYQQKPDQSPKLLVYAATQSFSGVPSRFSGSGSGSDYTLTINSLEAEDAAAYYCQHYYTTPPTFGPGTKVDIK

SEQ ID NO: 153 - protein, 107 aa, Artificial Sequence
  F8-L3 Light Chain Variable Region
EIVLTQSPDFQSVTPKEKVTITCRVSENIDSYLAWYQQKPDQSPKLLVYAATQSFSGVPSRFSGSGSGTDFTLTINSLEAEDAAAYYCQHYYTTPPTFGPGTKVDIK

SEQ ID NO: 154 - protein, 121 aa, Artificial Sequence
  F19-H1 Heavy Chain Variable Region
QVQLQESGPGLVRPSQTLSLTCTASGFTFSDYWMNWVRQPPGRGLEWVAFIGNKFHNYETEYNPSVKGRFTILRDDSKNQVSLRLSSVTAADTAVYYCTKLGRGYYVMDYWGQGSLVTVSS

SEQ ID NO: 155 - protein, 121 aa, Artificial Sequence
  F19-H2 Heavy Chain Variable Region
QVQLQESGPGLVKPSQTLSLTCTASGFTFSDYWMNWVRQPPGKGLEWVAFIGNKFHNYETEYNPSVKGRFTILRDDSKNQVSLKLSSVTAADTAVYYCTKLGRGYYVMDYWGQGSLVTVSS

SEQ ID NO: 156 - protein, 121 aa, Artificial Sequence
  F19-H3 Heavy Chain Variable Region
QVQLVESGGGVVQPGRSLRLSCAASGFTFSDYWMNWVRQAPGKGLEWVAIIGNKFHNYETYYADSVKGRFTISRDDSKNTVYLQMNGLRAEDTAVYYCTKLGRGYYVMDYWGQGTLVTVSS

SEQ ID NO: 157 - protein, 107 aa, Artificial Sequence
  F19-L1 Light Chain Variable Region
DIQMTQSPSSLSASVGDRVTITCKASQDITNHLNWFQQKPGKAFKLLIYYTSNLQTGVPSRFSGSGSGTDYTFTISSLQPEDIATYFCQQDSQHPWTFGQGTKVEIK

SEQ ID NO: 158 - protein, 107 aa, Artificial Sequence
  F19-L2 Light Chain Variable Region
EIVMTQSPDFQSVTPKEKVTITCRASQDITNHLNWFQQKPDQSFKLLIYYTSQSFSGVPSRFSGSGSGTDYTLTINSLEAEDAAAYFCQQDSQHPWTFGPGTKVDIK

SEQ ID NO: 159 - protein, 239 aa, Artificial Sequence
  F5-H2(G4S)3L2
EVQLVESGGGLVQPGGSLRLSCAVSGFTFSNYGMSWVRQAPGKGLEWVATISSGGSYTYYPDSVKGRFTISRDNSKNTLYLQMNSLRAEDTAVYYCSTQGSSGYVGYWGQGTLVTVSSGGGGSGGGGSGGGGSDIQMTQSPSSVSASVGDRVTITCKASQDITNFIGWYQHKPGKAPKLLISYTSILESGVPSRFSGSGSGTDYTLTISSLQPEDFATYYCLQYYNLWTFGGGTKVEIK

SEQ ID NO: 160 - protein, 244 aa, Artificial Sequence
  F5-H2(G4S)4L2
EVQLVESGGGLVQPGGSLRLSCAVSGFTFSNYGMSWVRQAPGKGLEWVATISSGGSYTYYPDSVKGRFTISRDNSKNTLYLQMNSLRAEDTAVYYCSTQGSSGYVGYWGQGTLVTVSSGGGGSGGGGSGGGGSGGGGSDIQMTQSPSSVSASVGDRVTITCKASQDITNFIGWYQHKPGKAPKLLISYTSILESGVPSRFSGSGSGTDYTLTISSLQPEDFATYYCLQYYNLWTFGGGTKVEIK

SEQ ID NO: 161 - protein, 239 aa, Artificial Sequence
  F5-L2(G4S)3H2
DIQMTQSPSSVSASVGDRVTITCKASQDITNFIGWYQHKPGKAPKLLISYTSILESGVPSRFSGSGSGTDYTLTISSLQPEDFATYYCLQYYNLWTFGGGTKVEIKGGGGSGGGGSGGGGSEVQLVESGGGLVQPGGSLRLSCAVSGFTFSNYGMSWVRQAPGKGLEWVATISSGGSYTYYPDSVKGRFTISRDNSKNTLYLQMNSLRAEDTAVYYCSTQGSSGYVGYWGQGTLVTVSS

SEQ ID NO: 162 - protein, 242 aa, Artificial Sequence
  F10-H1(G4S)3L2
EVQLVQSGAEVKKPGSSVKVSCKASGYAFSSSWMNWVRQAPGQGLEWIGRIYPGDGYTHYNGMFKGRASLTADKSTSTGYMELSSLRSEDTAVFFCTRHGDFPYWYFDVWGRGTLVTVSPGGGGSGGGGSGGGGSDIQMTQSPSTLSASVGDRVTITCRASENIDSYLAWYQQKPGRAPKLLVYAATNLAVGVPSRFSGSGSGTEYTLTISSLQPDDFATYYCQHHYSTPPTFGQGTKLEIK

SEQ ID NO: 163 - protein, 247 aa, Artificial Sequence
  F10-H1(G4S)4L2
EVQLVQSGAEVKKPGSSVKVSCKASGYAFSSSWMNWVRQAPGQGLEWIGRIYPGDGYTHYNGMFKGRASLTADKSTSTGYMELSSLRSEDTAVFFCTRHGDFPYWYFDVWGRGTLVTVSPGGGGSGGGGSGGGGSGGGGSDIQMTQSPSTLSASVGDRVTITCRASENIDSYLAWYQQKPGRAPKLLVYAATNLAVGVPSRFSGSGSGTEYTLTISSLQPDDFATYYCQHHYSTPPTFGQGTKLEIK

SEQ ID NO: 164 - protein, 242 aa, Artificial Sequence
  F10-L2(G4S)3H1
DIQMTQSPSTLSASVGDRVTITCRASENIDSYLAWYQQKPGRAPKLLVYAATNLAVGVPSRFSGSGSGTEYTLTISSLQPDDFATYYCQHHYSTPPTFGQGTKLEIKGGGGSGGGGSGGGGSEVQLVQSGAEVKKPGSSVKVSCKASGYAFSSSWMNWVRQAPGQGLEWIGRIYPGDGYTHYNGMFKGRASLTADKSTSTGYMELSSLRSEDTAVFFCTRHGDFPYWYFDVWGRGTLVTVSP

SEQ ID NO: 165 - protein, 240 aa, Artificial Sequence
  F17-H1(G4S)3L2
EVQLVETGGGLIQPGGSLRLSCAASGFTFSDFGMHWIRQAPGKGLEWVAYMSYTPGTFHYADTVKDRFTISRDNSKNTLYLQMNSLRAEDTAVYYCARVHVGTVDYWGQGTLVTVSSGGGGSGGGGSGGGGSEVVLTQSPATLSLSPGERATLSCRASQNINNNLHWFQQKPGQAPRLLIKYASQSISGIPARFSGSGSGTDFTLTISSLETEDFAVYFCQQSNSWPALTFGQGTKVEIK

SEQ ID NO: 166 - protein, 245 aa, Artificial Sequence
  F17-H1(G4S)4L2
EVQLVETGGGLIQPGGSLRLSCAASGFTFSDFGMHWIRQAPGKGLEWVAYMSYTPGTFHYADTVKDRFTISRDNSKNTLYLQMNSLRAEDTAVYYCARVHVGTVDYWGQGTLVTVSSGGGGSGGGGSGGGGSGGGGSEVVLTQSPATLSLSPGERATLSCRASQNINNNLHWFQQKPGQAPRLLIKYASQSISGIPARFSGSGSGTDFTLTISSLETEDFAVYFCQQSNSWPALTFGQGTKVEIK

SEQ ID NO: 167 - protein, 240 aa, Artificial Sequence
  F17-L2(G4S)3H1
EVVLTQSPATLSLSPGERATLSCRASQNINNNLHWFQQKPGQAPRLLIKYASQSISGIPARFSGSGSGTDFTLTISSLETEDFAVYFCQQSNSWPALTFGQGTKVEIKGGGGSGGGGSGGGGSEVQLVETGGGLIQPGGSLRLSCAASGFTFSDFGMHWIRQAPGKGLEWVAYMSYTPGTFHYADTVKDRFTISRDNSKNTLYLQMNSLRAEDTAVYYCARVHVGTVDYWGQGTLVTVSS

SEQ ID NO: 168 - protein, 242 aa, Artificial Sequence
  F20-H1(G4S)3L3
QVQLVQSGAEVKKPGSSVKVSCKASGYTFASYYLYWVRQAPGQGLEWIGEINPRSGGTNFNEKFKSRATVTVDKSTSTAYMELSSLRSEDTAVYYCSRSGRLRGFYTMDYWGQGTLVTVSSGGGGSGGGGSGGGGSDIQMTQSPSTLSASVGDRVTITCKAGENVGSYVSWYQQKPGKAPKLLIYGASNRYTGVPARFSGSGSATEFTLTISSLQPDDFATYYCGQTYRFLTFGQGTKVEVK

SEQ ID NO: 169 - protein, 247 aa, Artificial Sequence
  F20-H1(G4S)4L3
QVQLVQSGAEVKKPGSSVKVSCKASGYTFASYYLYWVRQAPGQGLEWIGEINPRSGGTNFNEKFKSRATVTVDKSTSTAYMELSSLRSEDTAVYYCSRSGRLRGFYTMDYWGQGTLVTVSSGGGGSGGGGSGGGGSGGGGSDIQMTQSPSTLSASVGDRVTITCKAGENVGSYVSWYQQKPGKAPKLLIYGASNRYTGVPARFSGSGSATEFTLTISSLQPDDFATYYCGQTYRFLTFGQGTKVEVK

SEQ ID NO: 170 - protein, 242 aa, Artificial Sequence
  F20-L3(G4S)3H1
DIQMTQSPSTLSASVGDRVTITCKAGENVGSYVSWYQQKPGKAPKLLIYGASNRYTGVPARFSGSGSATEFTLTISSLQPDDFATYYCGQTYRFLTFGQGTKVEVKGGGGSGGGGSGGGGSQVQLVQSGAEVKKPGSSVKVSCKASGYTFASYYLYWVRQAPGQGLEWIGEINPRSGGTNFNEKFKSRATVTVDKSTSTAYMELSSLRSEDTAVYYCSRSGRLRGFYTMDYWGQGTLVTVSS

SEQ ID NO: 171 - protein, 121 aa, Artificial Sequence
  F8-H4 Heavy Chain Variable Region
QVQLVQSGGGLVKPGGSLRLSCAASGFTLSTYAMSWVRQAPGKGLEWVAAISGGGGDTYYADSVKGRFTISRDNSKNTLYLQMSSLRAEDTAVYYCARQSHYGSSYYFDNWGQGTMVTVSS

SEQ ID NO: 172 - protein, 121 aa, Artificial Sequence
  F8-H5 Heavy Chain Variable Region
QVQLVQSGGGLVKPGGSLRLSCAASGFTLSTYAMSWVRQAPGKGLEWVAAISGGGGDTYHLDSVKGRFTISRDNSKNTLYLQMSSLRAEDTAVYYCARQSHYGSSYYFDNWGQGTMVTVSS

SEQ ID NO: 173 - protein, 121 aa, Artificial Sequence
  F8-H6 Heavy Chain Variable Region
EVQLVVSGGGLIQPGGSLRLSCAASGFTLSTYAMTWVRQAPGKGLEWVAVISGGGGDTYYADSVVGRFTISRDNSKNTLYLQMNSLRAEDTAVYYCARQSHYGSSYYFDNWGQGTLVTVSS

SEQ ID NO: 174 - protein, 121 aa, Artificial Sequence
  F19-H4 Heavy Chain Variable Region
EVVLVESGGGLVQPGGSLRLSCAASGFTFSDYWISWVRQAPGKGLEWVAHIGNKFHNYETDYADSVKGRFTISRDDSKNTVYLQMNSLRAEDTAVYYCTKLGRGYYVMDYWGQGTLVTVSS

SEQ ID NO: 175 - protein, 121 aa, Artificial Sequence
  F19-H5 Heavy Chain Variable Region
EVQLVESGGGLVQPGGSLRLSCAASGFTFSDYWISWVRQAPGKGLEWVAHIGNKFHNYETDYADSVKGRFTISKDDSKNTVYLQMNSLRAEDTAVYYCTKLGRGYYVMDYWGQGTLVTVSS

SEQ ID NO: 176 - protein, 121 aa, Artificial Sequence
  F19-H6 Heavy Chain Variable Region
QVQLVESGGGVVQPGRSLRLSCAASGFTFSDYWMHWVRQAPGKGLEWVAVIGNKFHNYETYYADSVKGRFTISRDDSKNTVYLQMNSLRAEDTAVYYCTKLGRGYYVMDYWGQGTTVTVSS

SEQ ID NO: 177 - protein, 121 aa, Artificial Sequence
  F19-H7 Heavy Chain Variable Region
EVQLVESGGGLVQPGGSLRLSCAASGFTFSDYWMNWVRQAPGKGLEWVAHIGNKFHNYETDYADSVKGRFTISKDDSKNTVYLQMNSLRAEDTAVYYCTKLGRGYYVMDYWGQGTLVTVSS

SEQ ID NO: 178 - protein, 121 aa, Artificial Sequence
  F19-H8 Heavy Chain Variable Region
QVQLVESGGGVVQPGRSLRLSCAASGFTFSDYWMHWVRQAPGKGLEWVAVIGNKFHNYETYYTDSVKGRFTISRDDSKNTVYLQMNTLRAEDTAVYYCTKLGRGYYVMDYWGKGTTVTVSS

SEQ ID NO: 179 - protein, 121 aa, Artificial Sequence
  F19-H9 Heavy Chain Variable Region
QVQLVESGGGVVQPGRSLRLSCAASGFTFSDYWMHWVRQAPGKGLEWVAVIGNKFHNYETYYTDSVKGRFTISRDNSKNTLYLQMNSLRAEDTAVYYCTKLGRGYYVMDYWGKGTTVTVSS

SEQ ID NO: 180 - protein, 121 aa, Artificial Sequence
  F19-H10 Heavy Chain Variable Region
QVVLVESGGGLVQPGGSLRLSCAASGFTFSDYWMHWVRQAPGKGLEWVASIGNKFHNYETYYADSVKGRFTISRDDSKNTVYLQMNSLFAEDTAVYYCTKLGRGYYVMDYWGQGTLVTVSS

SEQ ID NO: 181 - protein, 121 aa, Artificial Sequence
  F19-H11 Heavy Chain Variable Region
QVQLVESGGGVVQPGGSLRLSCAASGFTFSDYWMHWVRQAPGKGLEWVAFIGNKFHNYETYYADSVKGRFTISRDNSKNTLYLQMNSLRAEDTAVYYCTKLGRGYYVMDYWGQGTLVTVSS

SEQ ID NO: 182 - protein, 121 aa, Artificial Sequence
  F19-H12 Heavy Chain Variable Region
EVQLVESGGGLVQPGGSLRLSCVASGFTFSDYWMNWVRQAPGKGLEWVAQIGNKFHNYETYYSDSVKGRFTISKDDSKNTVYLQMNSLRAEDTAVYYCTKLGRGYYVMDYWGQGTLVTVSS

SEQ ID NO: 183 - protein, 118 aa, Artificial Sequence
  F5-H5 Heavy Chain Variable Region
QVELVESGGGVVQPGRSLRLDCKVSGFTFSNYGMHWVRQAPGKGLEWVAVISSGGSYTYYADSVKGRFTISRDNSKNTLFLQMNSLRAEDTAVYYCSTQGSSGYVGYWGQGTLVTVSS

SEQ ID NO: 184 - protein, 118 aa, Artificial Sequence
  F5-H6 Heavy Chain Variable Region
QVQLVESGGGVVQPGRSLRLSCKVSGFTFSNYGMHWVRQAPGKGLEWVAVISSGGSYTYYADSVKGRFTISRDNSKNTLFLQMNSLRAEDTAVYYCSTQGSSGYVGYWGQGTLVTVSS

SEQ ID NO: 185 - protein, 107 aa, Artificial Sequence
  F8-L4 Light Chain Variable Region
DIQMTQSPSSLSASVGDRVTITCRVSENIDSYLAWYQQKPGKAPKLLVYAATSLQSGVPSRFSGSGSGSDYTLTISSLQPEDFAVYYCQHYYTTPPTFGDGTKVEIK

SEQ ID NO: 186 - protein, 107 aa, Artificial Sequence
  F8-L5 Light Chain Variable Region
DIQMTQSPSSLSASVGDRVTITCRVSENIDSYLAWYQQKPGKAPKLLVYAATNLASGVPSRFSGSGSGSDYTLTISSLQPEDFAVYYCQHYYTTPPTFGDGTKVEIK

SEQ ID NO: 187 - protein, 107 aa, Artificial Sequence
  F8-L6 Light Chain Variable Region
DIQMTQSPSTLSASVGDRVTITCRVSENIDSYLAWYQQKPGKAPKLLVYAATSLESGVPSRFSGSGSGSEYTLTISSLQPDDFATYYCQHYYTTPPTFGQGTKVDIK

SEQ ID NO: 188 - protein, 107 aa, Artificial Sequence
  F19-L3 Light Chain Variable Region
DIQMTQSPSSLSASVGDRVTITCRASQDITNHLNWFQQKPGKAFKLLIYYTSSRASGVPSRFSGSGSGTDYTLTISSLQPEDFATYFCQQDSQHPWTFGQGTKVEIK

SEQ ID NO: 189 - protein, 107 aa, Artificial Sequence
  F19-L4 Light Chain Variable Region
DIQMTQSPSSLSASVGDRVTITCRASQDITNHLNWFQQKPGKAFKLLIYYTSSLQSGVPSRFSGSGSGTDYTLTISSLQPEDFATYFCQQDSQHPWTFGPGTKVEIK

SEQ ID NO: 190 - protein, 107 aa, Artificial Sequence
  F19-L5 Light Chain Variable Region
DIQMTQSPSSLSASVGDRVTITCRASQDITNHLNWFQQKPGKAFKLLIYYTSRLHSGVPSRFSGSGSGTDYTLTISSLQPEDFATYFCQQDSQHPWTFGGGTKVEIK

SEQ ID NO: 191 - protein, 107 aa, Artificial Sequence
  F19-L8 Light Chain Variable Region
DIQMTQSPSSLSASVGDRVTITCRASQDITNHLNWFQQKPGKAFKLLIYYTSSLQSGVPSRFSGSGSGTDYTLTISSLQPEDFATYFCQQDSQHPWTFGGGTKVEIK

SEQ ID NO: 192 - protein, 107 aa, Artificial Sequence
  F19-L9 Light Chain Variable Region
DIQMTQSPSSLSASVGDRVTITCRASQDITNHLNWYQQKPGKAPKLLIYYTSSLQSGVPSRFSGSGSGTDFTLTISSLQPEDFATYYCQQDSQHPWTFGGGTKVEIK

SEQ ID NO: 193 - protein, 107 aa, Artificial Sequence
  F19-L10 Light Chain Variable Region
EIVMTQSPATLSLSPGERATLSCRASQDITNHLNWFQQKPGQAFRLLIYYTSSRATGVPARFSGSGSGTDFTLTISSLEPEDFAVYFCQQDSQHPWTFGQGTKVEIK

SEQ ID NO: 194 - protein, 107 aa, Artificial Sequence
  F19-L11 Light Chain Variable Region
EIVLTQSPATLSLSPGERATLSCRASQDITNHLAWYQQKPGQAPRLLIYYTSNRATGIPARFSGSGSGTDFTLTISSLEPEDFAVYYCQQDSQHPWTFGQGTKVEIK

SEQ ID NO: 195 - protein, 106 aa, Artificial Sequence
  F5-L3 Light Chain Variable Region
AIQMTQSPSSLSASVGDRVTITCRASQDITNFLGWYQHKPGEAPKLLISYTSVLQSGVPSRFSGSGSGRDYTLTISSLQPEDFATYYCLQYYNLWTFGQGTKVEIK

SEQ ID NO: 196 - protein, 106 aa, Artificial Sequence
  F5-L4 Light Chain Variable Region
EIVMTQSPDFQSVTPKEKVTITCRASQDITNFIGWYQHKPDQSPKLLISYTSQSFSGVPSRFSGSGSGRDYTLTINSLEAEDAAAYYCLQYYNLWTFGPGTKVDIK

SEQ ID NO: 197 - protein, 106 aa, Artificial Sequence
  F5-L5 Light Chain Variable Region
EIVMTQSPATLSLSPGERATLSCRASQDITNFLGWYQHKPGQAPRLLISYTSNRATGIPARFSGSGSGRDYTLTISSLEPEDFAVYYCLQYYNLWTFGQGTKVEIK

SEQ ID NO: 198 - protein, 106 aa, Artificial Sequence
  F5-L6 Light Chain Variable Region
EIVMTQSPATLSLSPGERATLSCRASQDITNFLGWYQQKPGQAPRLLISYTSIRATGIPARFSGSGSGTDYTLTISSLEPEDFAVYYCLQYYNLWTFGQGTKVEIK

(19) United States
(12) Patent Application Publication
(10) Pub. No.: US 2022/0162301 A1
(43) Pub. Date: May 26, 2022

(54) HUMANIZED ANTI-FOLATE RECEPTOR 1 CHIMERIC ANTIGEN RECEPTORS AND USES THEREOF

(71) Applicant: PhanesTherapeutics, Inc., San Diego, CA (US)

(72) Inventors: Minghan Wang, San Diego, CA (US); Hui Zou, San Diego, CA (US); Haiqun Jia, San Diego, CA (US)

(21) Appl. No.: 17/593,094

(22) PCT Filed: Apr. 7, 2020
(86) PCT No.: PCT/US20/27092

§ 371 Date: Sep. 9, 2021

Related U.S. Application Data
(60) Provisional application No. 62/832,975, filed on Apr. 12, 2019.
(60) Provisional application No. 62/863,330, filed on Jun. 19, 2019.
(60) Provisional application No. 62/931,988, filed on Nov. 7, 2019.

(51) Int. Cl. C07K 16/28; A61K 35/17; A61K 45/06; A61K 39/395; A61K 38/17; C07K 14/705; C07K 14/725

(52) U.S. Cl. CPC C07K 16/28; C07K 2317/24; A61K 45/06; A61K 39/3955; A61K 38/1774; C07K 14/70517; C07K 14/70521; C07K 14/7051; C07K 2319/30; C07K 2319/33; C07K 2319/03; C07K 2319/02; C07K 2317/565; C07K 2317/622; A61K 35/17

(57) ABSTRACT

Chimeric antigen receptors (CARs) specific to FOLR1, vectors encoding the FOLR1 CAR, recombinant host cells comprising the FOLR1 CAR (CAR-Ts or CAR-NKs), and methods of using the CAR-Ts or CAR-NKs to treat a disease associated with the expression of FOLR1 thereof are described. Humanized anti-FOLR1 monoclonal antibodies and antigen-binding fragments thereof are also described.

### CROSS REFERENCE TO RELATED APPLICATIONS

This application claims priority to U.S. Provisional Application No. 62/832,975, filed on Apr. 12, 2019; U.S. Provisional Application No. 62/863,330, filed on Jun. 19, 2019; and U.S. Provisional Application No. 62/931,988, filed on Nov. 7, 2019. Each disclosure is incorporated herein by reference in its entirety.

### FIELD OF THE INVENTION

This invention relates to anti-folate receptor 1 (FOLR1) chimeric antigen receptors (CARs), nucleic acids and expression vectors encoding the CARs, T cells engineered to express the CARs (CAR-T) and NK cells engineered to express the CARs (CAR-NK). Methods of making the CARs, methods of making the CAR-Ts/CAR-NKs, and methods of using the CAR-Ts/CAR-NKs to treat a disease associated with the expression of FOLR1, including cancer, are also provided.

### REFERENCE TO SEQUENCE LISTING SUBMITTED ELECTRONICALLY

This application contains a sequence listing, which is submitted electronically via EFS-Web as an ASCII formatted sequence listing with a file name “065799.21WO1 Sequence Listing” and a creation date of Mar. 26, 2020 and having a size of 140 kb. The sequence listing submitted via EFS-Web is part of the specification and is herein incorporated by reference in its entirety.

### BACKGROUND OF THE INVENTION

The standard of care anti-cancer medicines provides significant benefits. Recently, the availability of immuno-oncology drugs such as anti-PD-1 mAbs, anti-PD-L1 mAbs and anti-CD3 bispecific T cell engagers has advanced the concept of leveraging and activating patients' immune system to fight various types of cancer. However, poor response, insufficient efficacy, and/or safety issues remain to be resolved. CAR-T (chimeric antigen receptor-T) cell therapies involve genetically engineering a patient's own immune cells, such as T cells, and redirecting them to a suitable cell surface antigen on cancer cells (Mayor et al., Immunotherapy. 2016; 8:491-494). This approach has demonstrated success in patients suffering from chemorefractory B cell malignancies and other cancers (Pettitt et al., Mol Ther. 2018; 26:342-353). T cells can be engineered to possess specificity to one or more cancer cell surface targets/antigens to recognize and kill the cancer cell. The process includes transducing T cells with DNA or other genetic material encoding the chimeric antigen receptor (CAR), which comprises an extracellular antigen specific binding domain, such as one or more single chain variable fragments (scFv) of a monoclonal antibody (mAb), a hinge and transmembrane region, and an intracellular signaling domain (including one or more costimulatory domains and one or more activating domains) (Kochenderfer et al., Nat Rev Clin Oncol. 2013; 10:267-276). CAR-expressing immune cells, such as T cells and NK cells, can be used to treat various diseases, including liquid and solid tumors. Successful CAR-T cell therapies can specifically recognize and destroy targeted cells and maintain the ability to persist and proliferate over time.

Folate receptor 1 (FOLR1), also known as folate receptor α (FRα) or folate binding protein (FBP), is a glycosylphosphatidylinositol (GPI)-anchored membrane protein on cell surface that has high affinity for and transports the active form of folate, 5-methyltetrahydrofolate (5-MTF), and its derivatives into cells (Salazar and Ratnam, Cancer Metastasis Rev 2007; 26:141-52). FOLR1 has become an oncology target because it is overexpressed in certain solid tumors such as ovarian, lung and breast cancers (Toffoli et al., Int J Cancer 1997; 74:193-198 and Boogerd et al., Oncotarget 2016; 7:17442-17454), but its expression is at low levels in limited normal human tissues (Weitman, et al., Cancer Res 1992; 52:3396-3401). Consistent with this observation, phase 1 clinical trials conducted so far with FOLR1-targeting small and large molecules revealed good drug tolerability (Cheung et al., Oncotarget 2016; 7:52553-52574). Therefore, FOLR1 is an ideal target for CAR-T cell therapies to treat and cure FOLR1-positive cancers.

### BRIEF SUMMARY OF THE INVENTION

In one general aspect, the invention relates to a chimeric antigen receptor (CAR) construct that induces T cell mediated cancer killing, wherein the CAR construct comprises at least one antigen binding domain that specifically binds human folate receptor 1 (FOLR1), a hinge region, a transmembrane region, and an intracellular signaling domain.

Provided are isolated polynucleotides comprising a nucleic acid sequence encoding a chimeric antigen receptor (CAR). The CAR can comprise (a) an extracellular domain comprising at least one antigen binding domain that specifically binds folate receptor 1 (FOLR1); (b) a hinge region; (c) a transmembrane region; and (d) an intracellular signaling domain.

In certain embodiments, the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:

(1) SEQ ID NOs: 17, 18, 19, 41, 42 and 43, respectively;

(2) SEQ ID NOs: 20, 21, 22, 44, 45 and 46, respectively;

(3) SEQ ID NOs: 23, 24, 25, 47, 48 and 49, respectively;

(4) SEQ ID NOs: 26, 27, 28, 50, 51 and 52, respectively;

(5) SEQ ID NOs: 29, 30, 31, 53, 54 and 55, respectively;

(6) SEQ ID NOs: 32, 33, 34, 56, 57 and 58, respectively;

(7) SEQ ID NOs: 35, 36, 37, 59, 60 and 61, respectively; or

(8) SEQ ID NOs: 38, 39, 40, 62, 63 and 64, respectively;

wherein the antigen binding domain specifically binds FOLR1, preferably human FOLR1.

In certain embodiments, the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:

(1) SEQ ID NOs: 65, 66, 67, 89, 90 and 91, respectively;

(2) SEQ ID NOs: 68, 69, 70, 92, 93 and 94, respectively;

(3) SEQ ID NOs: 71, 72, 73, 95, 96 and 97, respectively;

(4) SEQ ID NOs: 74, 75, 76, 98, 99 and 100, respectively;

(5) SEQ ID NOs: 77, 78, 79, 101, 102 and 103, respectively;

(6) SEQ ID NOs: 80, 81, 82, 104, 105 and 106, respectively;

(7) SEQ ID NOs: 83, 84, 85, 107, 108 and 109, respectively; or

(8) SEQ ID NOs: 86, 87, 88, 110, 111 and 112, respectively;

wherein the antigen binding domain specifically binds FOLR1, preferably human FOLR1.

In certain embodiments, the antigen binding domain comprises a heavy chain variable region having a polypeptide sequence at least 95%, at least 96%, at least 97%, at least 98%, or at least 99% identical to SEQ ID NO: 1, 3, 5, 7, 9, 11, 13, or 15, or a light chain variable region having a polypeptide sequence at least 95%, at least 96%, at least 97%, at least 98%, or at least 99% identical to SEQ ID NO: 2, 4, 6, 8, 10, 12, 14, or 16.

In certain embodiments, the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14; or
(8) a heavy chain variable region having the polypeptide sequence of SEQ ID NO:15, and a light chain variable region having the polypeptide sequence of SEQ ID NO:16.

In certain embodiments, the antigen binding domain is humanized and comprises a heavy chain variable region having a polypeptide sequence at least 95%, at least 96%, at least 97%, at least 98%, or at least 99% identical to SEQ ID NO: 113, 114, 115, 116, 119, 120, 124, 125, 128, 129, 130, 136, 137, 138, 142, 143, 144, 148, 149, 150, 154, 155, 156 or 171-184, or a light chain variable region having a polypeptide sequence at least 95%, at least 96%, at least 97%, at least 98%, or at least 99% identical to SEQ ID NO: 117, 118, 121, 122, 123, 126, 127, 131, 132, 133, 134, 139, 140, 141, 145, 146, 147, 151, 152, 153, 157, 158 or 185-198.

In certain embodiments, the antigen binding domain is humanized and comprises:
(1) a heavy chain variable region having the polypeptide sequence of SEQ ID NO:113, and a light chain variable region having the polypeptide sequence of SEQ ID NO:117;
(2) a heavy chain variable region having the polypeptide sequence of SEQ ID NO:113, and a light chain variable region having the polypeptide sequence of SEQ ID NO:118;
(3) a heavy chain variable region having the polypeptide sequence of SEQ ID NO:114, and a light chain variable region having the polypeptide sequence of SEQ ID NO:117;
(4) a heavy chain variable region having the polypeptide sequence of SEQ ID NO:114, and a light chain variable region having the polypeptide sequence of SEQ ID NO:118;
(5) a heavy chain variable region having the polypeptide sequence of SEQ ID NO:115, and a light chain variable region having the polypeptide sequence of SEQ ID NO:117;
(6) a heavy chain variable region having the polypeptide sequence of SEQ ID NO:115, and a light chain variable region having the polypeptide sequence of SEQ ID NO:118;
(7) a heavy chain variable region having the polypeptide sequence of SEQ ID NO:116, and a light chain variable region having the polypeptide sequence of SEQ ID NO:117;
(8) a heavy chain variable region having the polypeptide sequence of SEQ ID NO:116, and a light chain variable region having the polypeptide sequence of SEQ ID NO:118;
(9) a heavy chain variable region having the polypeptide sequence of SEQ ID NO:119, and a light chain variable region having the polypeptide sequence of SEQ ID NO:121;
(10) a heavy chain variable region having the polypeptide sequence of SEQ ID NO:119, and a light chain variable region having the polypeptide sequence of SEQ ID NO:122;
(11) a heavy chain variable region having the polypeptide sequence of SEQ ID NO:119, and a light chain variable region having the polypeptide sequence of SEQ ID NO:123;
(12) a heavy chain variable region having the polypeptide sequence of SEQ ID NO:120, and a light chain variable region having the polypeptide sequence of SEQ ID NO:121;
(13) a heavy chain variable region having the polypeptide sequence of SEQ ID NO:120, and a light chain variable region having the polypeptide sequence of SEQ ID NO:122;
(14) a heavy chain variable region having the polypeptide sequence of SEQ ID NO:120, and a light chain variable region having the polypeptide sequence of SEQ ID NO:123;
(15) a heavy chain variable region having the polypeptide sequence of SEQ ID NO:124, and a light chain variable region having the polypeptide sequence of SEQ ID NO:126;
(16) a heavy chain variable region having the polypeptide sequence of SEQ ID NO:124, and a light chain variable region having the polypeptide sequence of SEQ ID NO:127;
(17) a heavy chain variable region having the polypeptide sequence of SEQ ID NO:128, and a light chain variable region having the polypeptide sequence of SEQ ID NO:131;
(18) a heavy chain variable region having the polypeptide sequence of SEQ ID NO:128, and a light chain variable region having the polypeptide sequence of SEQ ID NO:132;
(19) a heavy chain variable region having the polypeptide sequence of SEQ ID NO:128, and a light chain variable region having the polypeptide sequence of SEQ ID NO:133;
(20) a heavy chain variable region having the polypeptide sequence of SEQ ID NO:128, and a light chain variable region having the polypeptide sequence of SEQ ID NO:134;
(21) a heavy chain variable region having the polypeptide sequence of SEQ ID NO:129, and a light chain variable region having the polypeptide sequence of SEQ ID NO:131;
(22) a heavy chain variable region having the polypeptide sequence of SEQ ID NO:129, and a light chain variable region having the polypeptide sequence of SEQ ID NO:132;
(23) a heavy chain variable region having the polypeptide sequence of SEQ ID NO:129, and a light chain variable region having the polypeptide sequence of SEQ ID NO:133;
(24) a heavy chain variable region having the polypeptide sequence of SEQ ID NO:129, and a light chain variable region having the polypeptide sequence of SEQ ID NO:134;
(25) a heavy chain variable region having the polypeptide sequence of SEQ ID NO:130, and a light chain variable region having the polypeptide sequence of SEQ ID NO:131;
(26) a heavy chain variable region having the polypeptide sequence of SEQ ID NO:130, and a light chain variable region having the polypeptide sequence of SEQ ID NO:132;
(27) a heavy chain variable region having the polypeptide sequence of SEQ ID NO:130, and a light chain variable region having the polypeptide sequence of SEQ ID NO:133;
(28) a heavy chain variable region having the polypeptide sequence of SEQ ID NO:130, and a light chain variable region having the polypeptide sequence of SEQ ID NO:134;
(29) a heavy chain variable region having the polypeptide sequence of SEQ ID NO:136, and a light chain variable region having the polypeptide sequence of SEQ ID NO:139;
(30) a heavy chain variable region having the polypeptide sequence of SEQ ID NO:136, and a light chain variable region having the polypeptide sequence of SEQ ID NO:140;
(31) a heavy chain variable region having the polypeptide sequence of SEQ ID NO:136, and a light chain variable region having the polypeptide sequence of SEQ ID NO:141;
(32) a heavy chain variable region having the polypeptide sequence of SEQ ID NO:137, and a light chain variable region having the polypeptide sequence of SEQ ID NO:139;
(33) a heavy chain variable region having the polypeptide sequence of SEQ ID NO:137, and a light chain variable region having the polypeptide sequence of SEQ ID NO:140;
(34) a heavy chain variable region having the polypeptide sequence of SEQ ID NO:137, and a light chain variable region having the polypeptide sequence of SEQ ID NO:141;
(35) a heavy chain variable region having the polypeptide sequence of SEQ ID NO:138, and a light chain variable region having the polypeptide sequence of SEQ ID NO:139;
(36) a heavy chain variable region having the polypeptide sequence of SEQ ID NO:138, and a light chain variable region having the polypeptide sequence of SEQ ID NO:140;
(37) a heavy chain variable region having the polypeptide sequence of SEQ ID NO:138, and a light chain variable region having the polypeptide sequence of SEQ ID NO:141;
(38) a heavy chain variable region having the polypeptide sequence of SEQ ID NO:142, and a light chain variable region having the polypeptide sequence of SEQ ID NO:145;
(39) a heavy chain variable region having the polypeptide sequence of SEQ ID NO:142, and a light chain variable region having the polypeptide sequence of SEQ ID NO:146;
(40) a heavy chain variable region having the polypeptide sequence of SEQ ID NO:142, and a light chain variable region having the polypeptide sequence of SEQ ID NO:147;
(41) a heavy chain variable region having the polypeptide sequence of SEQ ID NO:143, and a light chain variable region having the polypeptide sequence of SEQ ID NO:145;
(42) a heavy chain variable region having the polypeptide sequence of SEQ ID NO:143, and a light chain variable region having the polypeptide sequence of SEQ ID NO:146;
(43) a heavy chain variable region having the polypeptide sequence of SEQ ID NO:143, and a light chain variable region having the polypeptide sequence of SEQ ID NO:147;
(44) a heavy chain variable region having the polypeptide sequence of SEQ ID NO:144, and a light chain variable region having the polypeptide sequence of SEQ ID NO:145;
(45) a heavy chain variable region having the polypeptide sequence of SEQ ID NO:144, and a light chain variable region having the polypeptide sequence of SEQ ID NO:146;
(46) a heavy chain variable region having the polypeptide sequence of SEQ ID NO:144, and a light chain variable region having the polypeptide sequence of SEQ ID NO:147;
(47) a heavy chain variable region having the polypeptide sequence of SEQ ID NO:148, and a light chain variable region having the polypeptide sequence of SEQ ID NO:151;
(48) a heavy chain variable region having the polypeptide sequence of SEQ ID NO:150, and a light chain variable region having the polypeptide sequence of SEQ ID NO:153;
(49) a heavy chain variable region having the polypeptide sequence of SEQ ID NO:171, and a light chain variable region having the polypeptide sequence of SEQ ID NO:185;
(50) a heavy chain variable region having the polypeptide sequence of SEQ ID NO:171, and a light chain variable region having the polypeptide sequence of SEQ ID NO:186;
(51) a heavy chain variable region having the polypeptide sequence of SEQ ID NO:171, and a light chain variable region having the polypeptide sequence of SEQ ID NO:187;
(52) a heavy chain variable region having the polypeptide sequence of SEQ ID NO:173, and a light chain variable region having the polypeptide sequence of SEQ ID NO:185;
(53) a heavy chain variable region having the polypeptide sequence of SEQ ID NO:173, and a light chain variable region having the polypeptide sequence of SEQ ID NO:186;
(54) a heavy chain variable region having the polypeptide sequence of SEQ ID NO:173, and a light chain variable region having the polypeptide sequence of SEQ ID NO:187;
(55) a heavy chain variable region having the polypeptide sequence of SEQ ID NO:182, and a light chain variable region having the polypeptide sequence of SEQ ID NO:190;
(56) a heavy chain variable region having the polypeptide sequence of SEQ ID NO:183, and a light chain variable region having the polypeptide sequence of SEQ ID NO:195;
(57) a heavy chain variable region having the polypeptide sequence of SEQ ID NO:183, and a light chain variable region having the polypeptide sequence of SEQ ID NO:196;
(58) a heavy chain variable region having the polypeptide sequence of SEQ ID NO:183, and a light chain variable region having the polypeptide sequence of SEQ ID NO:197;
(59) a heavy chain variable region having the polypeptide sequence of SEQ ID NO:183, and a light chain variable region having the polypeptide sequence of SEQ ID NO:198;
(60) a heavy chain variable region having the polypeptide sequence of SEQ ID NO:184, and a light chain variable region having the polypeptide sequence of SEQ ID NO:195;
(61) a heavy chain variable region having the polypeptide sequence of SEQ ID NO:184, and a light chain variable region having the polypeptide sequence of SEQ ID NO:196;
(62) a heavy chain variable region having the polypeptide sequence of SEQ ID NO:184, and a light chain variable region having the polypeptide sequence of SEQ ID NO:197; or
(63) a heavy chain variable region having the polypeptide sequence of SEQ ID NO:184, and a light chain variable region having the polypeptide sequence of SEQ ID NO:198.

In certain embodiments, the antigen binding domain is a single chain variable fragment (scFv) that specifically binds FOLR1, preferably human FOLR1.

In certain embodiments, the antigen binding domain is a humanized single chain variable fragment (scFv) that specifically binds FOLR1, preferably human FOLR1. In certain embodiments, the humanized single chain variable fragment (scFv) comprises a polypeptide sequence at least 95% identical to any one of SEQ ID NOs: 159-170.

In certain embodiments, the chimeric antigen receptor (CAR) comprises one or more antigen binding domains.

In certain embodiments, the intracellular signaling domain comprises one or more costimulatory domains and one or more activating domains.

Also provided are chimeric antigen receptors (CARs) encoded by the isolated polynucleotides of the invention.

Also provided are vectors comprising the isolated polynucleotides comprising nucleic acids encoding the CARs of the invention.

Also provided are host cells comprising the vectors of the invention.

In certain embodiments, the host cell is a T cell, preferably a human T cell. In certain embodiments, the host cell is a NK cell, preferably a human NK cell. The T cell or NK cell can, for example, be engineered to express the CAR of the invention to treat diseases such as cancer.

Also provided are methods of making a host cell expressing a chimeric antigen receptor (CAR) of the invention. The methods comprise transducing a T cell or a NK cell with a vector comprising the isolated nucleic acids encoding the CARs of the invention.

Also provided are methods of producing a CAR-T cell or CAR-NK cell of the invention. The methods comprise culturing T cells or NK cells comprising the isolated polynucleotide comprising a nucleic acid encoding a chimeric antigen receptor (CAR) of the invention under conditions to produce the CAR-T cell or CAR-NK cell, and recovering the CAR-T cell or CAR-NK cell.

Also provided are methods of generating a population of RNA-engineered cells comprising a chimeric antigen receptor (CAR) of the invention. The methods comprise contacting a cell with the isolated polynucleotide comprising a nucleic acid encoding a chimeric antigen receptor (CAR) of the invention, wherein the isolated polynucleotide is an in vitro transcribed RNA or synthetic RNA.

Also provided are methods of treating cancer in a subject in need thereof, comprising administering to the subject the CAR-T cells and/or CAR-NK cells of the invention. The cancer can be any liquid or solid cancer, for example, it can be selected from, but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In certain embodiments, the methods of treating cancer in a subject in need thereof further comprise administering to the subject in need thereof an agent that increases the efficacy of a cell expressing a CAR molecule.

In certain embodiments, the methods of treating cancer in a subject in need thereof further comprise administering to the subject in need thereof an agent that ameliorates one or more side effects associated with administration of a cell expressing a CAR molecule.

In certain embodiments, the methods of treating cancer in a subject in need thereof further comprise administering to the subject in need thereof an agent that treats the disease associated with FOLR1.

Also provided are humanized anti-FOLR1 monoclonal antibodies or antigen-binding fragments thereof, wherein the antibodies or antigen-binding fragments thereof comprise a heavy chain variable region having a polypeptide sequence at least 95% identical to any one of SEQ ID NO: 113, 114, 115, 116, 119, 120, 124, 125, 128, 129, 130, 136, 137, 138, 142, 143, 144, 148, 149, 150, 154, 155, 156 or 171-184, or a light chain variable region having a polypeptide sequence at least 95% identical to SEQ ID NO: 117, 118, 121, 122, 123, 126, 127, 131, 132, 133, 134, 139, 140, 141, 145, 146, 147, 151, 152, 153, 157, 158 or 185-198.

In certain embodiments, the humanized anti-FOLR1 monoclonal antibodies or antigen-binding fragments thereof comprise:
(1) a heavy chain variable region having the polypeptide sequence of SEQ ID NO:113, and a light chain variable region having the polypeptide sequence of SEQ ID NO:117;
(2) a heavy chain variable region having the polypeptide sequence of SEQ ID NO:113, and a light chain variable region having the polypeptide sequence of SEQ ID NO:118;
(3) a heavy chain variable region having the polypeptide sequence of SEQ ID NO:114, and a light chain variable region having the polypeptide sequence of SEQ ID NO:117;
(4) a heavy chain variable region having the polypeptide sequence of SEQ ID NO:114, and a light chain variable region having the polypeptide sequence of SEQ ID NO:118;
(5) a heavy chain variable region having the polypeptide sequence of SEQ ID NO:115, and a light chain variable region having the polypeptide sequence of SEQ ID NO:117;
(6) a heavy chain variable region having the polypeptide sequence of SEQ ID NO:115, and a light chain variable region having the polypeptide sequence of SEQ ID NO:118;
(7) a heavy chain variable region having the polypeptide sequence of SEQ ID NO:116, and a light chain variable region having the polypeptide sequence of SEQ ID NO:117;
(8) a heavy chain variable region having the polypeptide sequence of SEQ ID NO:116, and a light chain variable region having the polypeptide sequence of SEQ ID NO:118;
(9) a heavy chain variable region having the polypeptide sequence of SEQ ID NO:119, and a light chain variable region having the polypeptide sequence of SEQ ID NO:121;
(10) a heavy chain variable region having the polypeptide sequence of SEQ ID NO:119, and a light chain variable region having the polypeptide sequence of SEQ ID NO:122;
(11) a heavy chain variable region having the polypeptide sequence of SEQ ID NO:119, and a light chain variable region having the polypeptide sequence of SEQ ID NO:123;
(12) a heavy chain variable region having the polypeptide sequence of SEQ ID NO:120, and a light chain variable region having the polypeptide sequence of SEQ ID NO:121;
(13) a heavy chain variable region having the polypeptide sequence of SEQ ID NO:120, and a light chain variable region having the polypeptide sequence of SEQ ID NO:122;
(14) a heavy chain variable region having the polypeptide sequence of SEQ ID NO:120, and a light chain variable region having the polypeptide sequence of SEQ ID NO:123;
(15) a heavy chain variable region having the polypeptide sequence of SEQ ID NO:124, and a light chain variable region having the polypeptide sequence of SEQ ID NO:126;
(16) a heavy chain variable region having the polypeptide sequence of SEQ ID NO:124, and a light chain variable region having the polypeptide sequence of SEQ ID NO:127;
(17) a heavy chain variable region having the polypeptide sequence of SEQ ID NO:128, and a light chain variable region having the polypeptide sequence of SEQ ID NO:131;
(18) a heavy chain variable region having the polypeptide sequence of SEQ ID NO:128, and a light chain variable region having the polypeptide sequence of SEQ ID NO:132;
(19) a heavy chain variable region having the polypeptide sequence of SEQ ID NO:128, and a light chain variable region having the polypeptide sequence of SEQ ID NO:133;
(20) a heavy chain variable region having the polypeptide sequence of SEQ ID NO:128, and a light chain variable region having the polypeptide sequence of SEQ ID NO:134;
(21) a heavy chain variable region having the polypeptide sequence of SEQ ID NO:129, and a light chain variable region having the polypeptide sequence of SEQ ID NO:131;
(22) a heavy chain variable region having the polypeptide sequence of SEQ ID NO:129, and a light chain variable region having the polypeptide sequence of SEQ ID NO:132;
(23) a heavy chain variable region having the polypeptide sequence of SEQ ID NO:129, and a light chain variable region having the polypeptide sequence of SEQ ID NO:133;
(24) a heavy chain variable region having the polypeptide sequence of SEQ ID NO:129, and a light chain variable region having the polypeptide sequence of SEQ ID NO:134;
(25) a heavy chain variable region having the polypeptide sequence of SEQ ID NO:130, and a light chain variable region having the polypeptide sequence of SEQ ID NO:131;
(26) a heavy chain variable region having the polypeptide sequence of SEQ ID NO:130, and a light chain variable region having the polypeptide sequence of SEQ ID NO:132;
(27) a heavy chain variable region having the polypeptide sequence of SEQ ID NO:130, and a light chain variable region having the polypeptide sequence of SEQ ID NO:133;
(28) a heavy chain variable region having the polypeptide sequence of SEQ ID NO:130, and a light chain variable region having the polypeptide sequence of SEQ ID NO:134;
(29) a heavy chain variable region having the polypeptide sequence of SEQ ID NO:136, and a light chain variable region having the polypeptide sequence of SEQ ID NO:139;
(30) a heavy chain variable region having the polypeptide sequence of SEQ ID NO:136, and a light chain variable region having the polypeptide sequence of SEQ ID NO:140;
(31) a heavy chain variable region having the polypeptide sequence of SEQ ID NO:136, and a light chain variable region having the polypeptide sequence of SEQ ID NO:141;
(32) a heavy chain variable region having the polypeptide sequence of SEQ ID NO:137, and a light chain variable region having the polypeptide sequence of SEQ ID NO:139;
(33) a heavy chain variable region having the polypeptide sequence of SEQ ID NO:137, and a light chain variable region having the polypeptide sequence of SEQ ID NO:140;
(34) a heavy chain variable region having the polypeptide sequence of SEQ ID NO:137, and a light chain variable region having the polypeptide sequence of SEQ ID NO:141;
(35) a heavy chain variable region having the polypeptide sequence of SEQ ID NO:138, and a light chain variable region having the polypeptide sequence of SEQ ID NO:139;
(36) a heavy chain variable region having the polypeptide sequence of SEQ ID NO:138, and a light chain variable region having the polypeptide sequence of SEQ ID NO:140;
(37) a heavy chain variable region having the polypeptide sequence of SEQ ID NO:138, and a light chain variable region having the polypeptide sequence of SEQ ID NO:141;
(38) a heavy chain variable region having the polypeptide sequence of SEQ ID NO:142, and a light chain variable region having the polypeptide sequence of SEQ ID NO:145;
(39) a heavy chain variable region having the polypeptide sequence of SEQ ID NO:142, and a light chain variable region having the polypeptide sequence of SEQ ID NO:146;
(40) a heavy chain variable region having the polypeptide sequence of SEQ ID NO:142, and a light chain variable region having the polypeptide sequence of SEQ ID NO:147;
(41) a heavy chain variable region having the polypeptide sequence of SEQ ID NO:143, and a light chain variable region having the polypeptide sequence of SEQ ID NO:145;
(42) a heavy chain variable region having the polypeptide sequence of SEQ ID NO:143, and a light chain variable region having the polypeptide sequence of SEQ ID NO:146;
(43) a heavy chain variable region having the polypeptide sequence of SEQ ID NO:143, and a light chain variable region having the polypeptide sequence of SEQ ID NO:147;
(44) a heavy chain variable region having the polypeptide sequence of SEQ ID NO:144, and a light chain variable region having the polypeptide sequence of SEQ ID NO:145;
(45) a heavy chain variable region having the polypeptide sequence of SEQ ID NO:144, and a light chain variable region having the polypeptide sequence of SEQ ID NO:146;
(46) a heavy chain variable region having the polypeptide sequence of SEQ ID NO:144, and a light chain variable region having the polypeptide sequence of SEQ ID NO:147;
(47) a heavy chain variable region having the polypeptide sequence of SEQ ID NO:148, and a light chain variable region having the polypeptide sequence of SEQ ID NO:151;
(48) a heavy chain variable region having the polypeptide sequence of SEQ ID NO:150, and a light chain variable region having the polypeptide sequence of SEQ ID NO:153;
(49) a heavy chain variable region having the polypeptide sequence of SEQ ID NO:171, and a light chain variable region having the polypeptide sequence of SEQ ID NO:185;
(50) a heavy chain variable region having the polypeptide sequence of SEQ ID NO:171, and a light chain variable region having the polypeptide sequence of SEQ ID NO:186;
(51) a heavy chain variable region having the polypeptide sequence of SEQ ID NO:171, and a light chain variable region having the polypeptide sequence of SEQ ID NO:187;
(52) a heavy chain variable region having the polypeptide sequence of SEQ ID NO:173, and a light chain variable region having the polypeptide sequence of SEQ ID NO:185;
(53) a heavy chain variable region having the polypeptide sequence of SEQ ID NO:173, and a light chain variable region having the polypeptide sequence of SEQ ID NO:186;
(54) a heavy chain variable region having the polypeptide sequence of SEQ ID NO:173, and a light chain variable region having the polypeptide sequence of SEQ ID NO:187;
(55) a heavy chain variable region having the polypeptide sequence of SEQ ID NO:182, and a light chain variable region having the polypeptide sequence of SEQ ID NO:190;
(56) a heavy chain variable region having the polypeptide sequence of SEQ ID NO:183, and a light chain variable region having the polypeptide sequence of SEQ ID NO:195;
(57) a heavy chain variable region having the polypeptide sequence of SEQ ID NO:183, and a light chain variable region having the polypeptide sequence of SEQ ID NO:196;
(58) a heavy chain variable region having the polypeptide sequence of SEQ ID NO:183, and a light chain variable region having the polypeptide sequence of SEQ ID NO:197;
(59) a heavy chain variable region having the polypeptide sequence of SEQ ID NO:183, and a light chain variable region having the polypeptide sequence of SEQ ID NO:198;
(60) a heavy chain variable region having the polypeptide sequence of SEQ ID NO:184, and a light chain variable region having the polypeptide sequence of SEQ ID NO:195;
(61) a heavy chain variable region having the polypeptide sequence of SEQ ID NO:184, and a light chain variable region having the polypeptide sequence of SEQ ID NO:196;
(62) a heavy chain variable region having the polypeptide sequence of SEQ ID NO:184, and a light chain variable region having the polypeptide sequence of SEQ ID NO:197; or
(63) a heavy chain variable region having the polypeptide sequence of SEQ ID NO:184, and a light chain variable region having the polypeptide sequence of SEQ ID NO:198.

In certain embodiments, the humanized anti-FOLR1 monoclonal antibody or antigen-binding fragment thereof is capable of binding FOLR1, inducing effector-mediated tumor cell lysis, mediating the recruitment of conjugated drugs, and/or forming a bispecific antibody with another monoclonal antibody or antigen-binding fragment with a cancer-killing effect.

Also provided are isolated nucleic acids encoding the humanized anti-FOLR1 monoclonal antibody or antigen-binding fragment thereof of the invention.

Also provided are vectors comprising the isolated nucleic acid encoding the humanized anti-FOLR1 monoclonal antibody or antigen-binding fragment thereof of the invention.

Also provided are host cells comprising the vector comprising the isolated nucleic acid encoding the humanized anti-FOLR1 monoclonal antibody or antigen-binding fragment thereof of the invention.

Also provided is a pharmaceutical composition comprising the humanized anti-FOLR1 monoclonal antibody or antigen-binding fragment thereof of the invention and a pharmaceutically acceptable carrier.

Also provided are methods of targeting FOLR1 on a cancer cell surface in a subject in need thereof, comprising administering to the subject in need thereof a pharmaceutical composition comprising the humanized anti-FOLR1 monoclonal antibody or antigen-binding fragment thereof of the invention.

Also provided are methods of treating cancer in a subject in need thereof, comprising administering to the subject the pharmaceutical composition comprising the humanized anti-FOLR1 monoclonal antibody or antigen-binding fragment thereof of the invention. The cancer can be any liquid or solid cancer, for example, it can be selected from, but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Also provided are methods of producing the humanized anti-FOLR1 monoclonal antibody or antigen-binding fragment thereof of the invention, comprising culturing a cell comprising a nucleic acid encoding the monoclonal antibody or antigen-binding fragment under conditions to produce the monoclonal antibody or antigen-binding fragment, and recovering the antibody or antigen-binding fragment from the cell or culture.

Also provided are method of producing a pharmaceutical composition comprising the humanized anti-FOLR1 monoclonal antibody or antigen-binding fragment thereof of the invention, comprising combining the monoclonal antibody or antigen-binding fragment thereof with a pharmaceutically acceptable carrier to obtain the pharmaceutical composition.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-1L show the binding of humanized mAbs to immobilized recombinant human FOLR1 protein by ELISA.

FIGS. 2A-2E show the binding of humanized mAbs to SK-OV-3 cells. The experiment was carried out by FACS analysis.

FIGS. 3A-3G show the binding of humanized scFvs to immobilized recombinant human FOLR1 protein by ELISA.

FIGS. 4A-4G show the binding of humanized scFvs to SK-OV-3 cells. The experiment was carried out by FACS analysis.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

The words “right,” “left,” “lower,” and “upper” designate directions in the drawings to which reference is made.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., chimeric antigen receptors (CARs) comprising antigen binding domains specific for FOLR1 and polynucleotides that encode them, FOLR1 polypeptides and FOLR1 polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 1981; 2:482, by the homology alignment algorithm of Needleman & Wunsch, J. Mol. Biol. 1970; 48:443, by the search for similarity method of Pearson & Lipman, Proc. Nat'l. Acad. Sci. USA 1988; 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., 1995 Supplement (Ausubel)).

Examples of algorithms that are suitable for determining percent sequence identity and sequence similarity are the BLAST and BLAST 2.0 algorithms, which are described in Altschul et al., J. Mol. Biol. 1990; 215: 403-410 and Altschul et al., Nucleic Acids Res. 1997; 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 1989; 89:10915).

In addition to calculating percent sequence identity, the BLAST algorithm also performs a statistical analysis of the similarity between two sequences (see, e.g., Karlin & Altschul, Proc. Nat'l. Acad. Sci. USA 1993; 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been “isolated” thus include nucleic acids and proteins purified by standard purification methods. “Isolated” nucleic acids, peptides and proteins can be part of a composition and still be isolated if the composition is not part of the native environment of the nucleic acid, peptide, or protein. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

As used herein, the term “vector” is a replicon in which another nucleic acid segment can be operably inserted so as to bring about the replication or expression of the segment.

As used herein, the term “host cell” refers to a cell comprising a nucleic acid molecule of the invention. The “host cell” can be any type of cell, e.g., a primary cell, a cell in culture, or a cell from a cell line. In one embodiment, a “host cell” is a cell transfected or transduced with a nucleic acid molecule of the invention. In another embodiment, a “host cell” is a progeny or potential progeny of such a transfected or transduced cell. A progeny of a cell may or may not be identical to the parent cell, e.g., due to mutations or environmental influences that can occur in succeeding generations or integration of the nucleic acid molecule into the host cell genome.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the term “immune cell” or “immune effector cell” refers to a cell that is involved in an immune response, e.g., in the promotion of an immune effector response. Examples of immune cells include T cells, B cells, natural killer (NK) cells, mast cells, and myeloid-derived phagocytes. According to particular embodiments, the engineered immune cells are T cells, and are referred to as CAR-T cells because they are engineered to express CARs of the invention.

As used herein, the term “engineered immune cell” refers to an immune cell, also referred to as an immune effector cell, that has been genetically modified by the addition of extra genetic material in the form of DNA or RNA to the total genetic material of the cell. According to embodiments herein, the engineered immune cells have been genetically modified to express a CAR construct according to the invention.

### Chimeric Antigen Receptor (CAR)

As used herein, the term “chimeric antigen receptor” (CAR) refers to a recombinant polypeptide comprising at least an extracellular domain that binds specifically to an antigen or a target, a transmembrane domain and an intracellular T cell receptor-activating signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

In one aspect, the CAR comprises an antigen binding domain, a hinge region, a costimulatory domain, an activating domain and a transmembrane region. In one aspect, the CAR comprises an antigen binding domain, a hinge region, two costimulatory domains, an activating domain and a transmembrane region. In one aspect, the CAR comprises two antigen binding domains, a hinge region, a costimulatory domain, an activating domain and a transmembrane region. In one aspect, the CAR comprises two antigen binding domains, a hinge region, two costimulatory domains, an activating domain and a transmembrane region.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” refers to the part of a CAR that connects two adjacent domains of the CAR protein, e.g., the extracellular domain and the transmembrane domain.

As used herein, the term “transmembrane domain” refers to the portion of a CAR that extends across the cell membrane and anchors the CAR to cell membrane. It is sometimes referred to as “transmembrane region”.

### Costimulatory Domains

As used herein, chimeric antigen receptors can incorporate costimulatory (signaling) domains to increase their potency. A costimulatory (signaling) domain can be derived from a costimulatory molecule. Costimulatory molecules are cell surface molecules other than antigen receptors or their ligands that are required for an efficient immune response. Costimulatory domains can be derived from costimulatory molecules, which can include, but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, or fragments or any combination thereof.

### Activating Domains

As used herein, chimeric antigen receptors can comprise activating domains. Activating domains can include, but are not limited to, CD3. CD3 is an element of the T cell receptor on native T cells and has been shown to be an important intracellular activating element in CARs. In a preferred embodiment, the CD3 is CD3 zeta.

### Hinge Region

As described herein, the chimeric antigen receptor can comprise a hinge region. This is a portion of the extracellular domain, sometimes referred to as a “spacer” region. A variety of hinges can be employed in accordance with the invention, including costimulatory molecules, as discussed above, immunoglobulin (Ig) sequences, or other suitable molecules to achieve the desired special distance from the target cell. In some embodiments, the entire extracellular region comprises a hinge region.

### Transmembrane Region

As used herein, chimeric antigen receptors (CARs) can comprise a transmembrane region/domain. The CAR can be designed to comprise a transmembrane domain that is fused to the extracellular domain of the CAR. It can similarly be fused to the intracellular domain of the CAR. In one embodiment, the transmembrane domain that is naturally associated with one of the domains in a CAR is used. In some instances, the transmembrane domain can be selected or modified by amino acid substitution to avoid binding of such domains to the transmembrane domains of the same or different surface membrane proteins to minimize interactions with other members of the receptor complex. The transmembrane domain may be derived either from a natural or from a synthetic source. Where the source is natural, the domain may be derived from any membrane-bound or transmembrane protein. Transmembrane regions of particular use in this invention can be derived from (i.e. comprise or engineered from), but are not limited to, CD28, CD28T, OX40, 4-1BB/CD137, CD2, CD3 (alpha, beta, delta, epsilon, gamma, zeta), CD4, CD5, CD7, CD9, CD16, CD22, CD27, CD30, CD33, CD37, CD40, CD45, CD64, CD80, CD86, CD134, CD137, CD154, programmed death-1 (PD-1), inducible T cell costimulator (ICOS), lymphocyte function-associated antigen-1 (LFA-1; CD11a and CD18), CD247, CD276 (B7-H3), LIGHT (tumor necrosis factor superfamily member 14; TNFSF14), NKG2C, Ig alpha (CD79a), DAP10, Fc gamma receptor, MHC class I molecule, TNFR, integrin, signaling lymphocytic activation molecule, BTLA, Toll ligand receptor, ICAM-1, CDS, GITR, BAFFR, LIGHT, HVEM (LIGHTR), KIRDS2, SLAMF7, NKp80 (KLRF1), NKp44, NKp30, NKp46, CD19, CD8 alpha, CD8 beta, IL-2R beta, IL-2R gamma, IL-7R alpha, ITGA4, VLA1, CD49a, IA4, CD49D, ITGA6, VLA-6, CD49f, ITGAD, ITGAE, CD103, ITGAL, CD1a, CD1b, CD1c, CD1d, ITGAM, ITGAX, ITGB1, CD29, ITGB2 (CD18), ITGB7, NKG2D, TNFR2, TRANCE/RANKL, DNAM1 (CD226), SLAMF4 (CD244, 2B4), CD84, CD96 (Tactile), CEACAM1, CRTAM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD83 ligand, cytokine receptor, activating NK cell receptors, an immunoglobulin protein, or fragments or any combination thereof.

### Immune Cells

According to particular aspects, the invention provides cells that are immune cells that comprise the isolated polynucleotides or vectors comprising the isolated polynucleotides comprising the nucleotide sequence encoding the CAR are provided herein. The immune cells comprising the isolated polynucleotides and/or vectors of the invention can be referred to as “engineered immune cells.” Preferably, the engineered immune cells are derived from a human (are of human origin prior to being made recombinant).

The engineered immune cells can, for example, be cells of the lymphoid lineage. Non-limiting examples of cells of the lymphoid lineage can include T cells and Natural Killer (NK) cells. T cells express the T cell receptor (TCR), with most cells expressing α and β chains and a smaller population expressing γ and δ chains. T cells useful as engineered immune cells of the invention can be CD4⁺ or CD8⁺ and can include, but are not limited to, T helper cells (CD4⁺), cytotoxic T cells (also referred to as cytotoxic T lymphocytes, CTL; CD8⁺ cells), and memory T cells, including central memory T cells, stem-like memory T cells, and effector memory T cells, natural killer T cells, mucosal associated invariant T cells, and γδ T cells. Other exemplary immune cells include, but are not limited to, macrophages, antigen presenting cells (APCs), or any immune cell that expresses an inhibitor of a cell-mediated immune response, for example, an immune checkpoint inhibitor pathway receptor (e.g., PD-1). Precursor cells of immune cells that can be used according to the invention, include, hematopoietic stem and/or progenitor cells. Hematopoietic stem and/or progenitor cells can be derived from bone marrow, umbilical cord blood, adult peripheral blood after cytokine mobilization, and the like, by methods known in the art. The immune cells are engineered to recombinantly express the CARs of the invention.

Immune cells and precursor cells thereof can be isolated by methods known in the art, including commercially available methods (see, e.g., Rowland Jones et al., Lymphocytes: A Practical Approach, Oxford University Press, N Y 1999). Sources for immune cells or precursors thereof include, but are not limited to, peripheral blood, umbilical cord blood, bone marrow, or other sources of hematopoietic cells. Various techniques can be employed to separate the cells to isolated or enrich desired immune cells. For instance, negative selection methods can be used to remove cells that are not the desired immune cells. Additionally, positive selection methods can be used to isolate or enrich for the desired immune cells or precursors thereof, or a combination of positive and negative selection methods can be employed. If a particular type of cell is to be isolated, e.g., a particular T cell, various cell surface markers or combinations of markers (e.g., CD3, CD4, CD8, CD34) can be used to separate the cells.

The immune cells or precursor cells thereof can be autologous or non-autologous to the subject to which they are administered in the methods of treatment of the invention. Autologous cells are isolated from the subject to which the engineered immune cells recombinantly expressing the CAR are to be administered. Optionally, the cells can be obtained by leukapheresis, where leukocytes are selectively removed from withdrawn blood, made recombinant, and then retransfused into the donor. Alternatively, allogeneic cells from a non-autologous donor that is not the subject can be used. In the case of a non-autologous donor, the cells are typed and matched for human leukocyte antigen (HLA) to determine the appropriate level of compatibility. For both autologous and non-autologous cells, the cells can optionally be cryopreserved until ready for use.

Various methods for isolating immune cells that can be used for recombinant expression of the CARs of the invention have been described previously, and can be used, including, but not limited to, using peripheral donor lymphocytes (Sadelain et al., Nat. Rev. Cancer 2003; 3:35-45; Morgan et al., Science 2006; 314:126-9), using lymphocyte cultures derived from tumor infiltrating lymphocytes (TILs) in tumor biopsies (Panelli et al., J. Immunol. 2000; 164:495-504; Panelli et al., J. Immunol. 2000; 164:4382-92) and using selectively in vitro expanded antigen-specific peripheral blood leukocytes employing artificial antigen-presenting cells (AAPCs) or dendritic cells (Dupont et al., Cancer Res. 2005; 65:5417-427; Papanicolaou et al., Blood 2003; 102:2498-505). In the case of using stem cells, the cells can be isolated by methods well known in the art (see, e.g., Klug et al., Hematopoietic Stem Cell Protocols, Humana Press, N J 2002; Freshney et al., Culture of Human Stem Cells, John Wiley & Sons 2007).

According to particular embodiments, the method of making the engineered immune cells comprises transfecting or transducing immune effector cells isolated from an individual such that the immune effector cells express one or more CAR(s) according to embodiments of the invention. Methods of preparing immune cells for immunotherapy are described, e.g., in WO2014/130635, WO2013/176916 and WO2013/176915, which are incorporated herein by reference. Individual steps that can be used for preparing engineered immune cells are disclosed, e.g., in WO2014/039523, WO2014/184741, WO2014/191128, WO2014/184744 and WO2014/184143, which are incorporated herein by reference.

In a particular embodiment, the immune effector cells, such as T cells, are genetically modified with CARs of the invention (e.g., transduced with a viral vector comprising a nucleic acid encoding a CAR) and then are activated and expanded in vitro. In various embodiments, T cells can be activated and expanded before or after genetic modification to express a CAR, using methods as described, for example, in U.S. Pat. Nos. 6,352,694, 6,534,055, 6,905,680, 6,692,964, 5,858,358, 6,887,466, 6,905,681, 7,144,575, 7,067,318, 7,172,869, 7,232,566, 7,175,843, 5,883,223, 6,905,874, 6,797,514, 6,867,041, US2006/121005, which are incorporated herein by reference. T cells can be expanded in vitro or in vivo. Generally, the T cells of the invention can be expanded by contact with a surface having attached thereto an agent that stimulates a CD3/TCR complex-associated signal and a ligand that stimulates a co-stimulatory molecule on the surface of the T cells. As non-limiting examples, T cell populations can be stimulated as described herein, such as by contact with an anti-CD3 antibody, or antigen-binding fragment thereof, or an anti-CD3 antibody immobilized on a surface, or by contact with a protein kinase C activator (e.g., bryostatin) in conjunction with a calcium ionophore, or by activation of the CAR itself. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include, e.g., an appropriate media (e.g., Minimal Essential Media or RPMI Media 1640 or, X-vivo 5 (Lonza)) that can contain factors necessary for proliferation and viability, including serum (e.g., fetal bovine or human serum), cytokines, such as IL-2, IL-7, IL-15, and/or IL-21, insulin, IFN-g, GM-CSF, TGFβ and/or any other additives for the growth of cells known to the skilled artisan. In other embodiments, the T cells can be activated and stimulated to proliferate with feeder cells and appropriate antibodies and cytokines using methods such as those described in U.S. Pat. Nos. 6,040,177, 5,827,642, and WO2012129514, which are incorporated herein by reference.

### Antibodies and Antigen Binding Domains

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to FOLR1 is substantially free of antibodies that do not bind to FOLR1). In addition, an isolated antibody is substantially free of other cellular material and/or chemicals.

As used herein, the term “monoclonal antibody” or “mAb” refers to an antibody obtained from a population of substantially homogeneous antibodies, i.e., the individual antibodies comprising the population are identical except for possible naturally occurring mutations that may be present in minor amounts. The monoclonal antibodies of the invention can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” and/or “antigen binding domain” refers to an antibody fragment such as, for example, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen binding domain is capable of binding to the same antigen to which the parent antibody binds. According to particular embodiments, the antigen binding domain comprises a single-chain antibody molecule (scFv).

As used herein, the term “single-chain antibody” refers to a conventional single-chain antibody in the field, which comprises a heavy chain variable region and a light chain variable region connected by a short peptide of about 5 to about 20 amino acids. As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” and/or “humanized antigen binding domain” refers to a non-human antibody and/or non-human antigen binding domain that is modified to increase the sequence homology to that of a human antibody and/or a human antigen binding domain, such that the antigen-binding properties of the antigen binding domain are retained, but its antigenicity in the human body is reduced.

As used herein, the term “chimeric antibody” and/or “chimeric antigen binding domain” refers to an antibody and/or antigen binding domain wherein the amino acid sequence of the immunoglobulin molecule is derived from two or more species. The variable region of both the light and heavy chains often corresponds to the variable region of an antibody and/or antigen binding domain derived from one species of mammal (e.g., mouse, rat, rabbit, etc.) having the desired specificity, affinity, and capability, while the constant regions correspond to the sequences of an antibody and/or antigen binding domain derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecifc antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, the first epitope is located on FOLR1 and the second epitope is located on PD-1, PD-L1, TIM-3, LAG-3, CD73, apelin, CTLA-4, EGFR, HER-2, CD3, CD19, CD20, CD33, CD47, TIP-1, CLDN18.2, DLL3, and/or other tumor associated immune suppressors or surface antigens.

As used herein, the term “FOLR1” refers to folate receptor 1 (FOLR1), also known as folate receptor α (FRα) or folate binding protein (FBP), which is a glycosylphosphatidylinositol (GPI)-anchored membrane protein on a cell surface that has high affinity for and transports the active form of folate, 5-methyltetrahydrofolate (5-MTF), and its derivatives into cells (Salazar and Ratnam, Cancer Metastasis Rev 2007; 26:141-52). FOLR1 has become an oncology target because it is overexpressed in certain solid tumors such as ovarian, lung and breast cancers (Toffoli et al., Int J Cancer 1997; 74:193-198 and Boogerd et al., Oncotarget 2016; 7:17442-17454), but its expression is at low levels in limited normal human tissues (Weitman, et al., Cancer Res 1992; 52:3396-3401). Consistent with this observation, phase 1 clinical trials conducted so far with FOLR1-targeted small and large molecules revealed good drug tolerability (Cheung et al., Oncotarget 2016; 7:52553-52574). Therefore, FOLR1 is an ideal target for CAR-T cell therapies to treat and cure FOLR1-positive cancers. An exemplary amino acid sequence of a human FOLR1 is represented by GenBank Accession No. NP_057937 (SEQ ID NO:135).

As used herein, an antibody and/or antigen binding domain that “specifically binds to FOLR1” refers to an antibody and/or antigen binding domain that binds to a FOLR1, preferably a human FOLR1, with a KD of 1×10⁻⁷ M or less, preferably 1×10⁻⁸ M or less, more preferably 5×10⁻⁹ M or less, 1×10⁻⁹ M or less, 5×10⁻¹⁹ M or less, or 1×10⁻¹⁹ M or less. The term “KD” refers to the dissociation constant, which is obtained from the ratio of Kd to Ka (i.e., Kd/Ka) and is expressed as a molar concentration (M). KD values for antigen binding domains can be determined using methods in the art in view of the present disclosure. For example, the KD of an antibody and/or antigen binding domain can be determined by using surface plasmon resonance, such as by using a biosensor system, e.g., a Biacore® system, or by using bio-layer interferometry technology, such as an Octet RED96 system.

The smaller the value of the KD of an antibody and/or antigen binding domain, the higher affinity that the antibody and/or antigen binding domain binds to a target antigen.

According to a particular aspect, the invention relates to chimeric antigen receptors (CAR)s comprising an antigen binding domain, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:

(1) SEQ ID NOs: 17, 18, 19, 41, 42 and 43, respectively;

(2) SEQ ID NOs: 20, 21, 22, 44, 45 and 46, respectively;

(3) SEQ ID NOs: 23, 24, 25, 47, 48 and 49, respectively;

(4) SEQ ID NOs: 26, 27, 28, 50, 51 and 52, respectively;

(5) SEQ ID NOs: 29, 30, 31, 53, 54 and 55, respectively;

(6) SEQ ID NOs: 32, 33, 34, 56, 57 and 58, respectively;

(7) SEQ ID NOs: 35, 36, 37, 59, 60 and 61, respectively; or

(8) SEQ ID NOs: 38, 39, 40, 62, 63 and 64, respectively;

wherein the antigen binding domain specifically binds FOLR1, preferably human FOLR1.

According to another particular aspect, the invention relates to chimeric antigen receptors (CARs) comprising an antigen binding domain, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:

(1) SEQ ID NOs: 65, 66, 67, 89, 90 and 91, respectively;

(2) SEQ ID NOs: 68, 69, 70, 92, 93 and 94, respectively;

(3) SEQ ID NOs: 71, 72, 73, 95, 96 and 97, respectively;

(4) SEQ ID NOs: 74, 75, 76, 98, 99 and 100, respectively;

(5) SEQ ID NOs: 77, 78, 79, 101, 102 and 103, respectively;

(6) SEQ ID NOs: 80, 81, 82, 104, 105 and 106, respectively;

(7) SEQ ID NOs: 83, 84, 85, 107, 108 and 109, respectively; or

(8) SEQ ID NOs: 86, 87, 88, 110, 111 and 112, respectively;

wherein the antigen binding domain specifically binds FOLR1, preferably human FOLR1.

According to another particular aspect, the invention relates to an antigen binding domain comprising a heavy chain variable region having a polypeptide sequence at least 95%, at least 96%, at least 97%, at least 98%, or at least 99% identical to SEQ ID NO: 1, 3, 5, 7, 9, 11, 13, or 15, or a light chain variable region having a polypeptide sequence at least 95%, at least 96%, at least 97%, at least 98%, or at least 99% identical to SEQ ID NO: 2, 4, 6, 8, 10, 12, 14, or 16.

According to another particular aspect, the invention relates to an antigen binding domain, comprising:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14; or
(8) a heavy chain variable region having the polypeptide sequence of SEQ ID NO:15, and a light chain variable region having the polypeptide sequence of SEQ ID NO:16.

According to another particular aspect, the antigen binding domain is humanized and comprises a heavy chain variable region having a polypeptide sequence at least 95%, at least 96%, at least 97%, at least 98%, or at least 99% identical to SEQ ID NO: 113, 114, 115, 116, 119, 120, 124, 125, 128, 129, 130, 136, 137, 138, 142, 143, 144, 148, 149, 150, 154, 155, 156 or 171-184, or a light chain variable region having a polypeptide sequence at least 95%, at least 96%, at least 97%, at least 98%, or at least 99% identical to SEQ ID NO: 117, 118, 121, 122, 123, 126, 127, 131, 132, 133, 134, 139, 140, 141, 145, 146, 147, 151, 152, 153, 157, 158 or 185-198.

According to another particular aspect, the antigen binding domain is humanized and comprises:
(1) a heavy chain variable region having the polypeptide sequence of SEQ ID NO:113, and a light chain variable region having the polypeptide sequence of SEQ ID NO: 17;
(2) a heavy chain variable region having the polypeptide sequence of SEQ ID NO:113, and a light chain variable region having the polypeptide sequence of SEQ ID NO:118;
(3) a heavy chain variable region having the polypeptide sequence of SEQ ID NO:114, and a light chain variable region having the polypeptide sequence of SEQ ID NO: 17;
(4) a heavy chain variable region having the polypeptide sequence of SEQ ID NO:114, and a light chain variable region having the polypeptide sequence of SEQ ID NO:118;
(5) a heavy chain variable region having the polypeptide sequence of SEQ ID NO:115, and a light chain variable region having the polypeptide sequence of SEQ ID NO: 17;
(6) a heavy chain variable region having the polypeptide sequence of SEQ ID NO:115, and a light chain variable region having the polypeptide sequence of SEQ ID NO:118;
(7) a heavy chain variable region having the polypeptide sequence of SEQ ID NO:116, and a light chain variable region having the polypeptide sequence of SEQ ID NO:117;
(8) a heavy chain variable region having the polypeptide sequence of SEQ ID NO:116, and a light chain variable region having the polypeptide sequence of SEQ ID NO:118;
(9) a heavy chain variable region having the polypeptide sequence of SEQ ID NO:119, and a light chain variable region having the polypeptide sequence of SEQ ID NO:121;
(10) a heavy chain variable region having the polypeptide sequence of SEQ ID NO:119, and a light chain variable region having the polypeptide sequence of SEQ ID NO:122;
(11) a heavy chain variable region having the polypeptide sequence of SEQ ID NO:119, and a light chain variable region having the polypeptide sequence of SEQ ID NO:123;
(12) a heavy chain variable region having the polypeptide sequence of SEQ ID NO:120, and a light chain variable region having the polypeptide sequence of SEQ ID NO:121;
(13) a heavy chain variable region having the polypeptide sequence of SEQ ID NO:120, and a light chain variable region having the polypeptide sequence of SEQ ID NO:122;
(14) a heavy chain variable region having the polypeptide sequence of SEQ ID NO:120, and a light chain variable region having the polypeptide sequence of SEQ ID NO:123;
(15) a heavy chain variable region having the polypeptide sequence of SEQ ID NO:124, and a light chain variable region having the polypeptide sequence of SEQ ID NO:126;
(16) a heavy chain variable region having the polypeptide sequence of SEQ ID NO:124, and a light chain variable region having the polypeptide sequence of SEQ ID NO:127;
(17) a heavy chain variable region having the polypeptide sequence of SEQ ID NO:128, and a light chain variable region having the polypeptide sequence of SEQ ID NO:131;
(18) a heavy chain variable region having the polypeptide sequence of SEQ ID NO:128, and a light chain variable region having the polypeptide sequence of SEQ ID NO:132;
(19) a heavy chain variable region having the polypeptide sequence of SEQ ID NO:128, and a light chain variable region having the polypeptide sequence of SEQ ID NO:133;
(20) a heavy chain variable region having the polypeptide sequence of SEQ ID NO:128, and a light chain variable region having the polypeptide sequence of SEQ ID NO:134;
(21) a heavy chain variable region having the polypeptide sequence of SEQ ID NO:129, and a light chain variable region having the polypeptide sequence of SEQ ID NO:131;
(22) a heavy chain variable region having the polypeptide sequence of SEQ ID NO:129, and a light chain variable region having the polypeptide sequence of SEQ ID NO:132;
(23) a heavy chain variable region having the polypeptide sequence of SEQ ID NO:129, and a light chain variable region having the polypeptide sequence of SEQ ID NO:133;
(24) a heavy chain variable region having the polypeptide sequence of SEQ ID NO:129, and a light chain variable region having the polypeptide sequence of SEQ ID NO:134;
(25) a heavy chain variable region having the polypeptide sequence of SEQ ID NO:130, and a light chain variable region having the polypeptide sequence of SEQ ID NO:131;
(26) a heavy chain variable region having the polypeptide sequence of SEQ ID NO:130, and a light chain variable region having the polypeptide sequence of SEQ ID NO:132;
(27) a heavy chain variable region having the polypeptide sequence of SEQ ID NO:130, and a light chain variable region having the polypeptide sequence of SEQ ID NO:133;
(28) a heavy chain variable region having the polypeptide sequence of SEQ ID NO:130, and a light chain variable region having the polypeptide sequence of SEQ ID NO:134;
(29) a heavy chain variable region having the polypeptide sequence of SEQ ID NO:136, and a light chain variable region having the polypeptide sequence of SEQ ID NO:139;
(30) a heavy chain variable region having the polypeptide sequence of SEQ ID NO:136, and a light chain variable region having the polypeptide sequence of SEQ ID NO:140;
(31) a heavy chain variable region having the polypeptide sequence of SEQ ID NO:136, and a light chain variable region having the polypeptide sequence of SEQ ID NO:141;
(32) a heavy chain variable region having the polypeptide sequence of SEQ ID NO:137, and a light chain variable region having the polypeptide sequence of SEQ ID NO:139;
(33) a heavy chain variable region having the polypeptide sequence of SEQ ID NO:137, and a light chain variable region having the polypeptide sequence of SEQ ID NO:140;
(34) a heavy chain variable region having the polypeptide sequence of SEQ ID NO:137, and a light chain variable region having the polypeptide sequence of SEQ ID NO:141;
(35) a heavy chain variable region having the polypeptide sequence of SEQ ID NO:138, and a light chain variable region having the polypeptide sequence of SEQ ID NO:139;
(36) a heavy chain variable region having the polypeptide sequence of SEQ ID NO:138, and a light chain variable region having the polypeptide sequence of SEQ ID NO:140;
(37) a heavy chain variable region having the polypeptide sequence of SEQ ID NO:138, and a light chain variable region having the polypeptide sequence of SEQ ID NO:141;
(38) a heavy chain variable region having the polypeptide sequence of SEQ ID NO:142, and a light chain variable region having the polypeptide sequence of SEQ ID NO:145;
(39) a heavy chain variable region having the polypeptide sequence of SEQ ID NO:142, and a light chain variable region having the polypeptide sequence of SEQ ID NO:146;
(40) a heavy chain variable region having the polypeptide sequence of SEQ ID NO:142, and a light chain variable region having the polypeptide sequence of SEQ ID NO:147;
(41) a heavy chain variable region having the polypeptide sequence of SEQ ID NO:143, and a light chain variable region having the polypeptide sequence of SEQ ID NO:145;
(42) a heavy chain variable region having the polypeptide sequence of SEQ ID NO:143, and a light chain variable region having the polypeptide sequence of SEQ ID NO:146;
(43) a heavy chain variable region having the polypeptide sequence of SEQ ID NO:143, and a light chain variable region having the polypeptide sequence of SEQ ID NO:147;
(44) a heavy chain variable region having the polypeptide sequence of SEQ ID NO:144, and a light chain variable region having the polypeptide sequence of SEQ ID NO:145;
(45) a heavy chain variable region having the polypeptide sequence of SEQ ID NO:144, and a light chain variable region having the polypeptide sequence of SEQ ID NO:146;
(46) a heavy chain variable region having the polypeptide sequence of SEQ ID NO:144, and a light chain variable region having the polypeptide sequence of SEQ ID NO:147;
(47) a heavy chain variable region having the polypeptide sequence of SEQ ID NO:148, and a light chain variable region having the polypeptide sequence of SEQ ID NO:151;
(48) a heavy chain variable region having the polypeptide sequence of SEQ ID NO:150, and a light chain variable region having the polypeptide sequence of SEQ ID NO:153;
(49) a heavy chain variable region having the polypeptide sequence of SEQ ID NO:171, and a light chain variable region having the polypeptide sequence of SEQ ID NO:185;
(50) a heavy chain variable region having the polypeptide sequence of SEQ ID NO:171, and a light chain variable region having the polypeptide sequence of SEQ ID NO:186;
(51) a heavy chain variable region having the polypeptide sequence of SEQ ID NO:171, and a light chain variable region having the polypeptide sequence of SEQ ID NO:187;
(52) a heavy chain variable region having the polypeptide sequence of SEQ ID NO:173, and a light chain variable region having the polypeptide sequence of SEQ ID NO:185;
(53) a heavy chain variable region having the polypeptide sequence of SEQ ID NO:173, and a light chain variable region having the polypeptide sequence of SEQ ID NO:186;
(54) a heavy chain variable region having the polypeptide sequence of SEQ ID NO:173, and a light chain variable region having the polypeptide sequence of SEQ ID NO:187;
(55) a heavy chain variable region having the polypeptide sequence of SEQ ID NO:182, and a light chain variable region having the polypeptide sequence of SEQ ID NO:190;
(56) a heavy chain variable region having the polypeptide sequence of SEQ ID NO:183, and a light chain variable region having the polypeptide sequence of SEQ ID NO:195;
(57) a heavy chain variable region having the polypeptide sequence of SEQ ID NO:183, and a light chain variable region having the polypeptide sequence of SEQ ID NO:196;
(58) a heavy chain variable region having the polypeptide sequence of SEQ ID NO:183, and a light chain variable region having the polypeptide sequence of SEQ ID NO:197;
(59) a heavy chain variable region having the polypeptide sequence of SEQ ID NO:183, and a light chain variable region having the polypeptide sequence of SEQ ID NO:198;
(60) a heavy chain variable region having the polypeptide sequence of SEQ ID NO:184, and a light chain variable region having the polypeptide sequence of SEQ ID NO:195;
(61) a heavy chain variable region having the polypeptide sequence of SEQ ID NO:184, and a light chain variable region having the polypeptide sequence of SEQ ID NO:196;
(62) a heavy chain variable region having the polypeptide sequence of SEQ ID NO:184, and a light chain variable region having the polypeptide sequence of SEQ ID NO:197; or
(63) a heavy chain variable region having the polypeptide sequence of SEQ ID NO:184, and a light chain variable region having the polypeptide sequence of SEQ ID NO:198.

According to another particular aspect, the antigen binding domain is a single chain variable fragment (scFv) that specifically binds FOLR1, preferably human FOLR1.

In certain embodiments, the encoded antigen binding domain is a humanized single chain variable fragment (scFv) that specifically binds FOLR1, preferably human FOLR1. In certain embodiments, the antigen binding domain is a humanized single chain variable fragment (scFv) that specifically binds FOLR1, preferably human FOLR1. In certain embodiments, the humanized single chain variable fragment (scFv) comprises a polypeptide sequence at least 95%, at least 96%, at least 97%, at least 98%, or at least 99% identical to any one of SEQ ID NOs:159-170. In certain embodiments, the humanized single chain variable fragment (scFv) comprises a polypeptide sequence having an amino acid sequence selected from the group consisting of SEQ ID NOs:159-170.

According to another particular aspect, the chimeric antigen receptor comprises one or more antigen binding domains.

According to another particular aspect, the intracellular signaling domain comprises one or more costimulatory domains and one or more activating domains.

In another general aspect, the invention relates to an isolated polynucleotide comprising a nucleic acid encoding chimeric antigen receptor (CAR), wherein the CAR comprises an antigen binding domain thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding antigen binding domains thereof of the invention can be altered without changing the amino acid sequences of the proteins.

In another general aspect, the invention relates to a vector comprising the isolated polynucleotide comprising the nucleic acid encoding the CAR, wherein the CAR comprises an antigen binding domain thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an antigen binding domain thereof in the cell. Conventional cloning techniques or artificial gene synthesis can be used to generate a recombinant expression vector according to embodiments of the invention.

In another general aspect, the invention relates to a cell transduced with the vector comprising the isolated nucleic acids encoding the CARs of the invention. The term “transduced” or “transduction” refers to a process by which exogenous nucleic acid is transferred or introduced into the host cell. A “transduced” cell is one which has been transduced with exogenous nucleic acid. The cell includes the primary subject cell and its progeny. In certain embodiments, the cell is a CAR-T cell, preferably a human CAR-T cell, wherein the T cell is engineered to express the CAR of the invention to treat diseases such as cancer. In certain embodiments, the cell is a CAR-NK cell, preferably a human CAR-NK cell, wherein the NK cell engineered to express the CAR of the invention is used to treat diseases such as cancer.

In another general aspect, the invention relates to a method of making a CAR-T cell by transducing a T cell with a vector comprising the isolated nucleic acids encoding the CARs of the invention.

In another general aspect, the invention relates to a method of producing the CAR-T cell thereof of the invention, comprising culturing T cells comprising a nucleic acid encoding a chimeric antigen receptor (CAR) of the invention under conditions to produce the CAR-T cell, and recovering the CAR-T cell.

In another general aspect, the invention relates to a method of making a CAR-NK cell by transducing a NK cell with a vector comprising the isolated nucleic acids encoding the CARs of the invention.

In another general aspect, the invention relates to a method of producing a CAR-NK cell of the invention, comprising culturing NK cells comprising nucleic acids encoding the chimeric antigen receptor (CAR) thereof under conditions to produce the CAR-NK cell, and recovering the CAR-NK cell.

In another general aspect, the invention relates to a method of generating a population of RNA-engineered cells comprising a chimeric antigen receptor (CAR) of the invention. The methods comprise contacting a population of cells with isolated polynucleotides comprising a nucleic acid encoding a CAR of the invention, wherein the isolated polynucleotides are in vitro transcribed RNA or synthetic RNA.

In another general aspect, the invention relates to a humanized anti-FOLR1 monoclonal antibody or antigen-binding fragment thereof, wherein the antibody or antigen-binding fragment thereof comprises a heavy chain variable region having a polypeptide sequence at least 95% identical to any one of SEQ ID NO: 113, 114, 115, 116, 119, 120, 124, 125, 128, 129, 130, 136, 137, 138, 142, 143, 144, 148, 149, 150, 154, 155, 156 or 171-184, or a light chain variable region having a polypeptide sequence at least 95% identical to SEQ ID NO: 117, 118, 121, 122, 123, 126, 127, 131, 132, 133, 134, 139, 140, 141, 145, 146, 147, 151, 152, 153, 157, 158 or 185-198.

According to another particular aspect, the humanized anti-FOLR1 monoclonal antibody or antigen-binding fragment thereof comprises:
(1) a heavy chain variable region having the polypeptide sequence of SEQ ID NO:113, and a light chain variable region having the polypeptide sequence of SEQ ID NO:117;
(2) a heavy chain variable region having the polypeptide sequence of SEQ ID NO:113, and a light chain variable region having the polypeptide sequence of SEQ ID NO:118;
(3) a heavy chain variable region having the polypeptide sequence of SEQ ID NO:114, and a light chain variable region having the polypeptide sequence of SEQ ID NO:117;
(4) a heavy chain variable region having the polypeptide sequence of SEQ ID NO:114, and a light chain variable region having the polypeptide sequence of SEQ ID NO:118;
(5) a heavy chain variable region having the polypeptide sequence of SEQ ID NO:115, and a light chain variable region having the polypeptide sequence of SEQ ID NO:117;
(6) a heavy chain variable region having the polypeptide sequence of SEQ ID NO:115, and a light chain variable region having the polypeptide sequence of SEQ ID NO:118;
(7) a heavy chain variable region having the polypeptide sequence of SEQ ID NO:116, and a light chain variable region having the polypeptide sequence of SEQ ID NO:117;
(8) a heavy chain variable region having the polypeptide sequence of SEQ ID NO:116, and a light chain variable region having the polypeptide sequence of SEQ ID NO:118;
(9) a heavy chain variable region having the polypeptide sequence of SEQ ID NO:119, and a light chain variable region having the polypeptide sequence of SEQ ID NO:121;
(10) a heavy chain variable region having the polypeptide sequence of SEQ ID NO:119, and a light chain variable region having the polypeptide sequence of SEQ ID NO:122;
(11) a heavy chain variable region having the polypeptide sequence of SEQ ID NO:119, and a light chain variable region having the polypeptide sequence of SEQ ID NO:123;
(12) a heavy chain variable region having the polypeptide sequence of SEQ ID NO:120, and a light chain variable region having the polypeptide sequence of SEQ ID NO:121;
(13) a heavy chain variable region having the polypeptide sequence of SEQ ID NO:120, and a light chain variable region having the polypeptide sequence of SEQ ID NO:122;
(14) a heavy chain variable region having the polypeptide sequence of SEQ ID NO:120, and a light chain variable region having the polypeptide sequence of SEQ ID NO:123;
(15) a heavy chain variable region having the polypeptide sequence of SEQ ID NO:124, and a light chain variable region having the polypeptide sequence of SEQ ID NO:126;
(16) a heavy chain variable region having the polypeptide sequence of SEQ ID NO:124, and a light chain variable region having the polypeptide sequence of SEQ ID NO:127;
(17) a heavy chain variable region having the polypeptide sequence of SEQ ID NO:128, and a light chain variable region having the polypeptide sequence of SEQ ID NO:131;
(18) a heavy chain variable region having the polypeptide sequence of SEQ ID NO:128, and a light chain variable region having the polypeptide sequence of SEQ ID NO:132;
(19) a heavy chain variable region having the polypeptide sequence of SEQ ID NO:128, and a light chain variable region having the polypeptide sequence of SEQ ID NO:133;
(20) a heavy chain variable region having the polypeptide sequence of SEQ ID NO:128, and a light chain variable region having the polypeptide sequence of SEQ ID NO:134;
(21) a heavy chain variable region having the polypeptide sequence of SEQ ID NO:129, and a light chain variable region having the polypeptide sequence of SEQ ID NO:131;
(22) a heavy chain variable region having the polypeptide sequence of SEQ ID NO:129, and a light chain variable region having the polypeptide sequence of SEQ ID NO:132;
(23) a heavy chain variable region having the polypeptide sequence of SEQ ID NO:129, and a light chain variable region having the polypeptide sequence of SEQ ID NO:133;
(24) a heavy chain variable region having the polypeptide sequence of SEQ ID NO:129, and a light chain variable region having the polypeptide sequence of SEQ ID NO:134;
(25) a heavy chain variable region having the polypeptide sequence of SEQ ID NO:130, and a light chain variable region having the polypeptide sequence of SEQ ID NO:131;
(26) a heavy chain variable region having the polypeptide sequence of SEQ ID NO:130, and a light chain variable region having the polypeptide sequence of SEQ ID NO:132;
(27) a heavy chain variable region having the polypeptide sequence of SEQ ID NO:130, and a light chain variable region having the polypeptide sequence of SEQ ID NO:133;
(28) a heavy chain variable region having the polypeptide sequence of SEQ ID NO:130, and a light chain variable region having the polypeptide sequence of SEQ ID NO:134;
(29) a heavy chain variable region having the polypeptide sequence of SEQ ID NO:136, and a light chain variable region having the polypeptide sequence of SEQ ID NO:139;
(30) a heavy chain variable region having the polypeptide sequence of SEQ ID NO:136, and a light chain variable region having the polypeptide sequence of SEQ ID NO:140;
(31) a heavy chain variable region having the polypeptide sequence of SEQ ID NO:136, and a light chain variable region having the polypeptide sequence of SEQ ID NO:141;
(32) a heavy chain variable region having the polypeptide sequence of SEQ ID NO:137, and a light chain variable region having the polypeptide sequence of SEQ ID NO:139;
(33) a heavy chain variable region having the polypeptide sequence of SEQ ID NO:137, and a light chain variable region having the polypeptide sequence of SEQ ID NO:140;
(34) a heavy chain variable region having the polypeptide sequence of SEQ ID NO:137, and a light chain variable region having the polypeptide sequence of SEQ ID NO:141;
(35) a heavy chain variable region having the polypeptide sequence of SEQ ID NO:138, and a light chain variable region having the polypeptide sequence of SEQ ID NO:139;
(36) a heavy chain variable region having the polypeptide sequence of SEQ ID NO:138, and a light chain variable region having the polypeptide sequence of SEQ ID NO:140;
(37) a heavy chain variable region having the polypeptide sequence of SEQ ID NO:138, and a light chain variable region having the polypeptide sequence of SEQ ID NO:141;
(38) a heavy chain variable region having the polypeptide sequence of SEQ ID NO:142, and a light chain variable region having the polypeptide sequence of SEQ ID NO:145;
(39) a heavy chain variable region having the polypeptide sequence of SEQ ID NO:142, and a light chain variable region having the polypeptide sequence of SEQ ID NO:146;
(40) a heavy chain variable region having the polypeptide sequence of SEQ ID NO:142, and a light chain variable region having the polypeptide sequence of SEQ ID NO:147;
(41) a heavy chain variable region having the polypeptide sequence of SEQ ID NO:143, and a light chain variable region having the polypeptide sequence of SEQ ID NO:145;
(42) a heavy chain variable region having the polypeptide sequence of SEQ ID NO:143, and a light chain variable region having the polypeptide sequence of SEQ ID NO:146;
(43) a heavy chain variable region having the polypeptide sequence of SEQ ID NO:143, and a light chain variable region having the polypeptide sequence of SEQ ID NO:147;
(44) a heavy chain variable region having the polypeptide sequence of SEQ ID NO:144, and a light chain variable region having the polypeptide sequence of SEQ ID NO:145;
(45) a heavy chain variable region having the polypeptide sequence of SEQ ID NO:144, and a light chain variable region having the polypeptide sequence of SEQ ID NO:146;
(46) a heavy chain variable region having the polypeptide sequence of SEQ ID NO:144, and a light chain variable region having the polypeptide sequence of SEQ ID NO:147;
(47) a heavy chain variable region having the polypeptide sequence of SEQ ID NO:148, and a light chain variable region having the polypeptide sequence of SEQ ID NO:151;
(48) a heavy chain variable region having the polypeptide sequence of SEQ ID NO:150, and a light chain variable region having the polypeptide sequence of SEQ ID NO:153;
(49) a heavy chain variable region having the polypeptide sequence of SEQ ID NO:171, and a light chain variable region having the polypeptide sequence of SEQ ID NO:185;
(50) a heavy chain variable region having the polypeptide sequence of SEQ ID NO:171, and a light chain variable region having the polypeptide sequence of SEQ ID NO:186;
(51) a heavy chain variable region having the polypeptide sequence of SEQ ID NO:171, and a light chain variable region having the polypeptide sequence of SEQ ID NO:187;
(52) a heavy chain variable region having the polypeptide sequence of SEQ ID NO:173, and a light chain variable region having the polypeptide sequence of SEQ ID NO:185;
(53) a heavy chain variable region having the polypeptide sequence of SEQ ID NO:173, and a light chain variable region having the polypeptide sequence of SEQ ID NO:186;
(54) a heavy chain variable region having the polypeptide sequence of SEQ ID NO:173, and a light chain variable region having the polypeptide sequence of SEQ ID NO:187;
(55) a heavy chain variable region having the polypeptide sequence of SEQ ID NO:182, and a light chain variable region having the polypeptide sequence of SEQ ID NO:190;
(56) a heavy chain variable region having the polypeptide sequence of SEQ ID NO:183, and a light chain variable region having the polypeptide sequence of SEQ ID NO:195;
(57) a heavy chain variable region having the polypeptide sequence of SEQ ID NO:183, and a light chain variable region having the polypeptide sequence of SEQ ID NO:196;
(58) a heavy chain variable region having the polypeptide sequence of SEQ ID NO:183, and a light chain variable region having the polypeptide sequence of SEQ ID NO:197;
(59) a heavy chain variable region having the polypeptide sequence of SEQ ID NO:183, and a light chain variable region having the polypeptide sequence of SEQ ID NO:198;
(60) a heavy chain variable region having the polypeptide sequence of SEQ ID NO:184, and a light chain variable region having the polypeptide sequence of SEQ ID NO:195;
(61) a heavy chain variable region having the polypeptide sequence of SEQ ID NO:184, and a light chain variable region having the polypeptide sequence of SEQ ID NO:196;
(62) a heavy chain variable region having the polypeptide sequence of SEQ ID NO:184, and a light chain variable region having the polypeptide sequence of SEQ ID NO:197; or
(63) a heavy chain variable region having the polypeptide sequence of SEQ ID NO:184, and a light chain variable region having the polypeptide sequence of SEQ ID NO:198.

According to another particular aspect, the humanized anti-FOLR1 monoclonal antibody or antigen-binding fragment thereof is capable of binding FOLR1, inducing effector-mediated tumor cell lysis, mediating the recruitment of conjugated drugs, and/or forming a bispecific antibody with another monoclonal antibody or antigen-binding fragment with a cancer-killing effect.

In another general aspect, the invention relates to an isolated nucleic acid encoding the humanized anti-FOLR1 monoclonal antibody or antigen-binding fragment thereof of the invention.

In another general aspect, the invention relates to a vector comprising the isolated nucleic acid encoding the humanized anti-FOLR1 monoclonal antibody or antigen-binding fragment thereof of the invention.

In another general aspect, the invention relates to a host cell comprising the vector comprising the isolated nucleic acid encoding the humanized anti-FOLR1 monoclonal antibody or antigen-binding fragment thereof of the invention.

In another general aspect, the invention relates to a method of producing the humanized anti-FOLR1 monoclonal antibody or antigen-binding fragment thereof of the invention, comprising culturing a cell comprising a nucleic acid encoding the monoclonal antibody or antigen-binding fragment under conditions to produce the monoclonal antibody or antigen-binding fragment, and recovering the antibody or antigen-binding fragment from the cell or culture.

### Pharmaceutical Compositions

In another general aspect, the invention relates to a pharmaceutical composition comprising an isolated polynucleotide of the invention, an isolated polypeptide of the invention, a host cell of the invention, and/or an engineered immune cell of the invention and a pharmaceutically acceptable carrier.

In another general aspect, the invention relates to a pharmaceutical composition comprising a humanized anti-FOLR1 monoclonal antibody or antigen-binding fragment thereof of the invention and a pharmaceutically acceptable carrier.

The term “pharmaceutical composition” as used herein means a product comprising an isolated polynucleotide of the invention, an isolated polypeptide of the invention, a host cell of the invention, an engineered immune cell of the invention, and/or a humanized anti-FOLR1 monoclonal antibody or antigen-binding fragment of the invention together with a pharmaceutically acceptable carrier. Polynucleotides, polypeptides, host cells, engineered immune cells of the invention, and/or a humanized anti-FOLR1 monoclonal antibody or antigen-binding fragment of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in a polynucleotide, polypeptide, host cell, and/or engineered immune cell pharmaceutical composition can be used in the invention.

The formulation of pharmaceutically active ingredients with pharmaceutically acceptable carriers is known in the art, e.g., Remington: The Science and Practice of Pharmacy (e.g. 21st edition 2005, and any later editions). Non-limiting examples of additional ingredients include: buffers, diluents, solvents, tonicity regulating agents, preservatives, stabilizers, and chelating agents. One or more pharmaceutically acceptable carriers may be used in formulating the pharmaceutical compositions of the invention.

In another general aspect, the invention relates to a method of producing a pharmaceutical composition comprising the humanized anti-FOLR1 monoclonal antibody or antigen-binding fragment thereof of the invention, comprising combining the monoclonal antibody or antigen-binding fragment thereof with a pharmaceutically acceptable carrier to obtain the pharmaceutical composition.

### Methods of Use

In another general aspect, the invention relates to a method of treating a cancer in a subject in need thereof, comprising administering to the subject the CAR-T cells and/or CAR-NK cells of the invention. The cancer can, for example, be selected from but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

In another general aspect, the invention relates to a method of targeting FOLR1 on a cancer cell surface in a subject in need thereof, comprising administering to the subject in need thereof a pharmaceutical composition comprising the humanized anti-FOLR1 monoclonal antibody or antigen-binding fragment thereof of the invention.

In another general aspect, the invention relates to a method of treating cancer in a subject in need thereof, comprising administering to the subject the pharmaceutical composition comprising the humanized anti-FOLR1 monoclonal antibody or antigen-binding fragment thereof of the invention. The cancer can be any liquid or solid cancer, for example, it can be selected from, but not limited to, a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to embodiments of the invention, the CAR-T cell or CAR-NK cells comprise a therapeutically effective amount of the expressed CARs of the invention and the pharmaceutical compositions comprise a therapeutically effective amount of the humanized anti-FOLR1 monoclonal antibody or antigen-binding fragment thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to CARs, a therapeutically effective amount means an amount of the CAR molecule expressed in the transduced T cell or NK cell that modulates an immune response in a subject in need thereof. Also, as used herein with reference to CARs, a therapeutically effective amount means an amount of the CAR molecule expressed in the transduced T cell or NK cell that results in treatment of a disease, disorder, or condition; prevents or slows the progression of the disease, disorder, or condition; or reduces or completely alleviates symptoms associated with the disease, disorder, or condition.

As used herein with reference to CAR-T cell or CAR-NK cell, a therapeutically effective amount means an amount of the CAR-T cells or CAR-NK cells that modulates an immune response in a subject in need thereof. Also, as used herein with reference to CAR-T cell or CAR-NK cell, a therapeutically effective amount means an amount of the CAR-T cells or CAR-NK cells that results in treatment of a disease, disorder, or condition; prevents or slows the progression of the disease, disorder, or condition; or reduces or completely alleviates symptoms associated with the disease, disorder, or condition.

As used herein with reference to a humanized anti-FOLR1 monoclonal antibody or antigen-binding fragment thereof, a therapeutically effective amount means an amount of the humanized anti-FOLR1 monoclonal antibody or antigen-binding fragment thereof that modulates an immune response in a subject in need thereof. Also, as used herein with reference to a humanized anti-FOLR1 monoclonal antibody or antigen-binding fragment thereof, a therapeutically effective amount means an amount of the humanized anti-FOLR1 monoclonal antibody or antigen binding fragment thereof that results in treatment of a disease, disorder, or condition; prevents or slows the progression of the disease, disorder, or condition; or reduces or completely alleviates symptoms associated with the disease, disorder, or condition.

According to particular embodiments, the disease, disorder or condition to be treated is cancer, preferably a cancer selected from the group consisting of a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the invention can be administered in any convenient manner known to those skilled in the art. For example, the cells of the invention can be administered to the subject by aerosol inhalation, injection, ingestion, transfusion, implantation, and/or transplantation. The compositions comprising the cells of the invention can be administered transarterially, subcutaneously, intradermaly, intratumorally, intranodally, intramedullary, intramuscularly, intrapleurally, by intravenous (i.v.) injection, or intraperitoneally. In certain embodiments, the cells of the invention can be administered with or without lymphodepletion of the subject.

The pharmaceutical compositions comprising cells of the invention expressing CARs of the invention can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the invention in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the invention.

The cells of the invention can be administered in any physiologically acceptable vehicle. A cell population comprising cells of the invention can comprise a purified population of cells. Those skilled in the art can readily determine the cells in a cell population using various well known methods. The ranges in purity in cell populations comprising genetically modified cells of the invention can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the invention are generally administered as a dose based on cells per kilogram (cells/kg) of body weight of the subject to which the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the invention are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

According to particular embodiments, provided are compositions used in the treatment of a cancer. For cancer therapy, the provided compositions can be used in combination with another treatment including, but not limited to, a chemotherapy, an anti-CD20 mAb, an anti-TIM-3 mAb, an anti-LAG-3 mAb, an anti-EGFR mAb, an anti-HER-2 mAb, an anti-CD19 mAb, an anti-CD33 mAb, an anti-CD47 mAb, an anti-CD73 mAb, an anti-DLL-3 mAb, an anti-apelin mAb, an anti-TIP-1 mAb, an anti-Claudin18.2 mAb, an anti-CTLA-4 mAb, an anti-PD-L1 mAb, an anti-PD-1 mAb, other immuno-oncology drugs, an antiangiogenic agent, a radiation therapy, an antibody-drug conjugate (ADC), a targeted therapy, or other anticancer drugs.

According to particular embodiments, the methods of treating cancer in a subject in need thereof comprise administering to the subject the CAR-T cells and/or CAR-NK cells of the invention in combination with an agent that increases the efficacy of a cell expressing a CAR molecule. Such agents include, but are not limited to, an antibody fragment that binds to CD73, CD39, PD1, PD-L1, PD-L2, CTLA4, TIM3 or LAG3, or an adenosine A2a receptor antagonist.

According to particular embodiments, the methods of treating cancer in a subject in need thereof comprise administering to the subject the CAR-T cells and/or CAR-NK cells of the invention in combination with an agent that ameliorates one or more side effects associated with administration of a cell expressing a CAR molecule. Such agents include, but are not limited to, a steroid, an inhibitor of TNFα, or an inhibitor of IL-6.

According to particular embodiments, the methods of treating cancer in a subject in need thereof comprise administering to the subject the CAR-T cells and/or CAR-NK cells of the invention in combination with an agent that treats the disease associated with FOLR1. Such agents include, but are not limited to, an anti-FOLR1 monoclonal antibody or bispecific antibody.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject.

### Embodiments

The invention provides also the following non-limiting embodiments.

Embodiment 1 is an isolated polynucleotide comprising a nucleic acid sequence encoding a chimeric antigen receptor (CAR), wherein the CAR comprises: (a) an extracellular domain comprising at least one antigen binding domain that specifically binds Folate receptor 1 (FOLR1); (b) a hinge region; (c) a transmembrane region; and (d) an intracellular signaling domain.

Embodiment 2 is the isolated polynucleotide of embodiment 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:

(1) SEQ ID NOs: 17, 18, 19, 41, 42 and 43, respectively;

(2) SEQ ID NOs: 20, 21, 22, 44, 45 and 46, respectively;

(3) SEQ ID NOs: 23, 24, 25, 47, 48 and 49, respectively;

(4) SEQ ID NOs: 26, 27, 28, 50, 51 and 52, respectively;

(5) SEQ ID NOs: 29, 30, 31, 53, 54 and 55, respectively;

(6) SEQ ID NOs: 32, 33, 34, 56, 57 and 58, respectively;

(7) SEQ ID NOs: 35, 36, 37, 59, 60 and 61, respectively; or

(8) SEQ ID NOs: 38, 39, 40, 62, 63 and 64, respectively;

wherein the antigen binding domain specifically binds FOLR1, preferably human FOLR1.

Embodiment 3 is the isolated polynucleotide of embodiment 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:

(1) SEQ ID NOs: 65, 66, 67, 89, 90 and 91, respectively;

(2) SEQ ID NOs: 68, 69, 70, 92, 93 and 94, respectively;

(3) SEQ ID NOs: 71, 72, 73, 95, 96 and 97, respectively;

(4) SEQ ID NOs: 74, 75, 76, 98, 99 and 100, respectively;

(5) SEQ ID NOs: 77, 78, 79, 101, 102 and 103, respectively;

(6) SEQ ID NOs: 80, 81, 82, 104, 105 and 106, respectively;

(7) SEQ ID NOs: 83, 84, 85, 107, 108 and 109, respectively; or

(8) SEQ ID NOs: 86, 87, 88, 110, 111 and 112, respectively;

wherein the antigen binding domain specifically binds FOLR1, preferably human FOLR1.

Embodiment 4 is the isolated polynucleotide of any one of embodiments 1-3, wherein the antigen binding domain comprises a heavy chain variable region having a polypeptide sequence at least 95%, at least 96%, at least 97%, at least 98%, or at least 99% identical to SEQ ID NO: 1, 3, 5, 7, 9, 11, 13, or 15, or a light chain variable region having a polypeptide sequence at least 95%, at least 96%, at least 97%, at least 98%, or at least 99% identical to SEQ ID NO: 2, 4, 6, 8, 10, 12, 14, or 16.

Embodiment 5 is the isolated polynucleotide of any one of embodiments 1-4, wherein the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14; or
(8) a heavy chain variable region having the polypeptide sequence of SEQ ID NO:15, and a light chain variable region having the polypeptide sequence of SEQ ID NO:16.

Embodiment 6 is the isolated polynucleotide of any one of embodiments 1-4, wherein the antigen binding domain is humanized and comprises a heavy chain variable region having a polypeptide sequence at least 95%, at least 96%, at least 97%, at least 98%, or at least 99% identical to SEQ ID NO: 113, 114, 115, 116, 119, 120, 124, 125, 128, 129, 130, 136, 137, 138, 142, 143, 144, 148, 149, 150, 154, 155, 156 or 171-184, or a light chain variable region having a polypeptide sequence at least 95%, at least 96%, at least 97%, at least 98%, or at least 99% identical to SEQ ID NO: 117, 118, 121, 122, 123, 126, 127, 131, 132, 133, 134, 139, 140, 141, 145, 146, 147, 151, 152, 153, 157, 158 or 185-198.

Embodiment 7 is the isolated polynucleotide of embodiment 6, wherein the antigen binding domain is humanized and comprises:
(1) a heavy chain variable region having the polypeptide sequence of SEQ ID NO:113, and a light chain variable region having the polypeptide sequence of SEQ ID NO:117;
(2) a heavy chain variable region having the polypeptide sequence of SEQ ID NO:113, and a light chain variable region having the polypeptide sequence of SEQ ID NO:118;
(3) a heavy chain variable region having the polypeptide sequence of SEQ ID NO:114, and a light chain variable region having the polypeptide sequence of SEQ ID NO:117;
(4) a heavy chain variable region having the polypeptide sequence of SEQ ID NO:114, and a light chain variable region having the polypeptide sequence of SEQ ID NO:118;
(5) a heavy chain variable region having the polypeptide sequence of SEQ ID NO:115, and a light chain variable region having the polypeptide sequence of SEQ ID NO:117;
(6) a heavy chain variable region having the polypeptide sequence of SEQ ID NO:115, and a light chain variable region having the polypeptide sequence of SEQ ID NO:118;
(7) a heavy chain variable region having the polypeptide sequence of SEQ ID NO:116, and a light chain variable region having the polypeptide sequence of SEQ ID NO:117;
(8) a heavy chain variable region having the polypeptide sequence of SEQ ID NO:116, and a light chain variable region having the polypeptide sequence of SEQ ID NO:118;
(9) a heavy chain variable region having the polypeptide sequence of SEQ ID NO:119, and a light chain variable region having the polypeptide sequence of SEQ ID NO:121;
(10) a heavy chain variable region having the polypeptide sequence of SEQ ID NO:119, and a light chain variable region having the polypeptide sequence of SEQ ID NO:122;
(11) a heavy chain variable region having the polypeptide sequence of SEQ ID NO:119, and a light chain variable region having the polypeptide sequence of SEQ ID NO:123;
(12) a heavy chain variable region having the polypeptide sequence of SEQ ID NO:120, and a light chain variable region having the polypeptide sequence of SEQ ID NO:121; (13) a heavy chain variable region having the polypeptide sequence of SEQ ID NO:120, and a light chain variable region having the polypeptide sequence of SEQ ID NO:122;

(14) a heavy chain variable region having the polypeptide sequence of SEQ ID NO:120, and a light chain variable region having the polypeptide sequence of SEQ ID NO:123;
(15) a heavy chain variable region having the polypeptide sequence of SEQ ID NO:124, and a light chain variable region having the polypeptide sequence of SEQ ID NO:126;
(16) a heavy chain variable region having the polypeptide sequence of SEQ ID NO:124, and a light chain variable region having the polypeptide sequence of SEQ ID NO:127;
(17) a heavy chain variable region having the polypeptide sequence of SEQ ID NO:128, and a light chain variable region having the polypeptide sequence of SEQ ID NO:131;
(18) a heavy chain variable region having the polypeptide sequence of SEQ ID NO:128, and a light chain variable region having the polypeptide sequence of SEQ ID NO:132;
(19) a heavy chain variable region having the polypeptide sequence of SEQ ID NO:128, and a light chain variable region having the polypeptide sequence of SEQ ID NO:133;
(20) a heavy chain variable region having the polypeptide sequence of SEQ ID NO:128, and a light chain variable region having the polypeptide sequence of SEQ ID NO:134;
(21) a heavy chain variable region having the polypeptide sequence of SEQ ID NO:129, and a light chain variable region having the polypeptide sequence of SEQ ID NO:131;
(22) a heavy chain variable region having the polypeptide sequence of SEQ ID NO:129, and a light chain variable region having the polypeptide sequence of SEQ ID NO:132;
(23) a heavy chain variable region having the polypeptide sequence of SEQ ID NO:129, and a light chain variable region having the polypeptide sequence of SEQ ID NO:133;
(24) a heavy chain variable region having the polypeptide sequence of SEQ ID NO:129, and a light chain variable region having the polypeptide sequence of SEQ ID NO:134;
(25) a heavy chain variable region having the polypeptide sequence of SEQ ID NO:130, and a light chain variable region having the polypeptide sequence of SEQ ID NO:131;
(26) a heavy chain variable region having the polypeptide sequence of SEQ ID NO:130, and a light chain variable region having the polypeptide sequence of SEQ ID NO:132;
(27) a heavy chain variable region having the polypeptide sequence of SEQ ID NO:130, and a light chain variable region having the polypeptide sequence of SEQ ID NO:133;
(28) a heavy chain variable region having the polypeptide sequence of SEQ ID NO:130, and a light chain variable region having the polypeptide sequence of SEQ ID NO:134;
(29) a heavy chain variable region having the polypeptide sequence of SEQ ID NO:136, and a light chain variable region having the polypeptide sequence of SEQ ID NO:139;
(30) a heavy chain variable region having the polypeptide sequence of SEQ ID NO:136, and a light chain variable region having the polypeptide sequence of SEQ ID NO:140;
(31) a heavy chain variable region having the polypeptide sequence of SEQ ID NO:136, and a light chain variable region having the polypeptide sequence of SEQ ID NO:141;
(32) a heavy chain variable region having the polypeptide sequence of SEQ ID NO:137, and a light chain variable region having the polypeptide sequence of SEQ ID NO:139;
(33) a heavy chain variable region having the polypeptide sequence of SEQ ID NO:137, and a light chain variable region having the polypeptide sequence of SEQ ID NO:140;
(34) a heavy chain variable region having the polypeptide sequence of SEQ ID NO:137, and a light chain variable region having the polypeptide sequence of SEQ ID NO:141;
(35) a heavy chain variable region having the polypeptide sequence of SEQ ID NO:138, and a light chain variable region having the polypeptide sequence of SEQ ID NO:139;
(36) a heavy chain variable region having the polypeptide sequence of SEQ ID NO:138, and a light chain variable region having the polypeptide sequence of SEQ ID NO:140;
(37) a heavy chain variable region having the polypeptide sequence of SEQ ID NO:138, and a light chain variable region having the polypeptide sequence of SEQ ID NO:141;
(38) a heavy chain variable region having the polypeptide sequence of SEQ ID NO:142, and a light chain variable region having the polypeptide sequence of SEQ ID NO:145;
(39) a heavy chain variable region having the polypeptide sequence of SEQ ID NO:142, and a light chain variable region having the polypeptide sequence of SEQ ID NO:146;
(40) a heavy chain variable region having the polypeptide sequence of SEQ ID NO:142, and a light chain variable region having the polypeptide sequence of SEQ ID NO:147;
(41) a heavy chain variable region having the polypeptide sequence of SEQ ID NO:143, and a light chain variable region having the polypeptide sequence of SEQ ID NO:145;
(42) a heavy chain variable region having the polypeptide sequence of SEQ ID NO:143, and a light chain variable region having the polypeptide sequence of SEQ ID NO:146;
(43) a heavy chain variable region having the polypeptide sequence of SEQ ID NO:143, and a light chain variable region having the polypeptide sequence of SEQ ID NO:147;
(44) a heavy chain variable region having the polypeptide sequence of SEQ ID NO:144, and a light chain variable region having the polypeptide sequence of SEQ ID NO:145;
(45) a heavy chain variable region having the polypeptide sequence of SEQ ID NO:144, and a light chain variable region having the polypeptide sequence of SEQ ID NO:146;
(46) a heavy chain variable region having the polypeptide sequence of SEQ ID NO:144, and a light chain variable region having the polypeptide sequence of SEQ ID NO:147;
(47) a heavy chain variable region having the polypeptide sequence of SEQ ID NO:148, and a light chain variable region having the polypeptide sequence of SEQ ID NO:151;
(48) a heavy chain variable region having the polypeptide sequence of SEQ ID NO:150, and a light chain variable region having the polypeptide sequence of SEQ ID NO:153;
(49) a heavy chain variable region having the polypeptide sequence of SEQ ID NO:171, and a light chain variable region having the polypeptide sequence of SEQ ID NO:185;
(50) a heavy chain variable region having the polypeptide sequence of SEQ ID NO:171, and a light chain variable region having the polypeptide sequence of SEQ ID NO:186;
(51) a heavy chain variable region having the polypeptide sequence of SEQ ID NO:171, and a light chain variable region having the polypeptide sequence of SEQ ID NO:187;
(52) a heavy chain variable region having the polypeptide sequence of SEQ ID NO:173, and a light chain variable region having the polypeptide sequence of SEQ ID NO:185;
(53) a heavy chain variable region having the polypeptide sequence of SEQ ID NO:173, and a light chain variable region having the polypeptide sequence of SEQ ID NO:186;
(54) a heavy chain variable region having the polypeptide sequence of SEQ ID NO:173, and a light chain variable region having the polypeptide sequence of SEQ ID NO:187;
(55) a heavy chain variable region having the polypeptide sequence of SEQ ID NO:182, and a light chain variable region having the polypeptide sequence of SEQ ID NO:190;
(56) a heavy chain variable region having the polypeptide sequence of SEQ ID NO:183, and a light chain variable region having the polypeptide sequence of SEQ ID NO:195;
(57) a heavy chain variable region having the polypeptide sequence of SEQ ID NO:183, and a light chain variable region having the polypeptide sequence of SEQ ID NO:196;
(58) a heavy chain variable region having the polypeptide sequence of SEQ ID NO:183, and a light chain variable region having the polypeptide sequence of SEQ ID NO:197;
(59) a heavy chain variable region having the polypeptide sequence of SEQ ID NO:183, and a light chain variable region having the polypeptide sequence of SEQ ID NO:198;
(60) a heavy chain variable region having the polypeptide sequence of SEQ ID NO:184, and a light chain variable region having the polypeptide sequence of SEQ ID NO:195;
(61) a heavy chain variable region having the polypeptide sequence of SEQ ID NO:184, and a light chain variable region having the polypeptide sequence of SEQ ID NO:196;
(62) a heavy chain variable region having the polypeptide sequence of SEQ ID NO:184, and a light chain variable region having the polypeptide sequence of SEQ ID NO:197; or
(63) a heavy chain variable region having the polypeptide sequence of SEQ ID NO:184, and a light chain variable region having the polypeptide sequence of SEQ ID NO:198.

Embodiment 8 is the isolated polynucleotide of any one of embodiments 1-7, wherein the antigen binding domain is a single chain variable fragment (scFv) that specifically binds FOLR1, preferably human FOLR1.

Embodiment 9 is the isolated polynucleotide of embodiment 8, wherein the single chain variable fragment (scFv) is humanized.

Embodiment 10 is the isolated polynucleotide of embodiment 8 or 9, wherein the single chain variable fragment (scFv) comprises a polypeptide sequence at least 95% identical to any one of SEQ ID NOs:159-170.

Embodiment 11 is the isolated polynucleotide of any one of embodiments 1-10, wherein the chimeric antigen receptor (CAR) comprises one or more antigen binding domains.

Embodiment 12 is the isolated polynucleotide of any one of embodiments 1-11, wherein the intracellular signaling domain of the CAR comprises one or more costimulatory domains and one or more activating domains.

Embodiment 13 is a chimeric antigen receptor (CAR) encoded by the isolated polynucleotide of any one of embodiments 1-12.

Embodiment 14 is a vector comprising the isolated polynucleotide of any one of embodiments 1-12.

Embodiment 15 is a host cell comprising the vector of embodiment 14.

Embodiment 16 is the host cell of embodiment 15, wherein the cell is a CAR-T cell, preferably a human CAR-T cell.

Embodiment 17 is the host cell of embodiment 15, wherein the cell is a CAR-NK cell, preferably a human CAR-NK cell.

Embodiment 18 is a method of making a host cell expressing a chimeric antigen receptor (CAR), the method comprising transducing a T cell with the vector of embodiment 14.

Embodiment 19 is a method of producing a chimeric antigen receptor (CAR)-T cell, the method comprising culturing T cells comprising the isolated polynucleotide comprising a nucleic acid encoding a chimeric antigen receptor (CAR) of any one of embodiments 1-12 under conditions to produce the CAR-T cell and recovering the CAR-T cell.

Embodiment 20 is a method of making a host cell expressing a chimeric antigen receptor (CAR), the method comprising transducing a NK cell with the vector of embodiment 14.

Embodiment 21 is a method of producing a chimeric antigen receptor (CAR)-NK cell, the method comprising culturing NK cells comprising the isolated polynucleotide comprising a nucleic acid encoding a chimeric antigen receptor (CAR) of any one of embodiments 1-12 under conditions to produce the CAR-NK cell, and recovering the CAR-NK cell.

Embodiment 22 is a method of generating a cell comprising a chimeric antigen receptor (CAR), the method comprising contacting a cell with the isolated polynucleotide comprising a nucleic acid encoding a chimeric antigen receptor (CAR) of any one of embodiments 1-12, wherein the isolated polynucleotide is an in vitro transcribed RNA or synthetic RNA.

Embodiment 23 is a method of treating cancer in a subject in need thereof, the method comprising administering to the subject the host cell of any one of embodiments 15-17.

Embodiment 24 is the method of embodiment 23, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 25 is the method of embodiment 23 or 24, further comprising administering to the subject in need thereof an agent that increases the efficacy of a cell expressing a CAR.

Embodiment 26 is the method of embodiment 23 or 24, further comprising administering to the subject in need thereof an agent that ameliorates one or more side effects associated with administration of a cell expressing a CAR.

Embodiment 27 is the method of embodiment 23 or 24, further comprising administering to the subject in need thereof an agent that treats the disease associated with FOLR1.

Embodiment 28 is a humanized anti-FOLR1 monoclonal antibody or antigen-binding fragment thereof, wherein the antibody or antigen-binding fragment thereof comprises a heavy chain variable region having a polypeptide sequence at least 95% identical to any one of SEQ ID NO: 113, 114, 115, 116, 119, 120, 124, 125, 128, 129, 130, 136, 137, 138, 142, 143, 144, 148, 149, 150, 154, 155, 156 or 171-184, or a light chain variable region having a polypeptide sequence at least 95% identical to SEQ ID NO: 117, 118, 121, 122, 123, 126, 127, 131, 132, 133, 134, 139, 140, 141, 145, 146, 147, 151, 152, 153, 157, 158 or 185-198.

Embodiment 29 is the humanized anti-FOLR1 monoclonal antibody or antigen-binding fragment thereof of embodiment 28, wherein the antibody or antigen-binding fragment thereof comprises:
(1) a heavy chain variable region having the polypeptide sequence of SEQ ID NO:113, and a light chain variable region having the polypeptide sequence of SEQ ID NO:117;
(2) a heavy chain variable region having the polypeptide sequence of SEQ ID NO:113, and a light chain variable region having the polypeptide sequence of SEQ ID NO:118;
(3) a heavy chain variable region having the polypeptide sequence of SEQ ID NO:114, and a light chain variable region having the polypeptide sequence of SEQ ID NO:117;
(4) a heavy chain variable region having the polypeptide sequence of SEQ ID NO:114, and a light chain variable region having the polypeptide sequence of SEQ ID NO:118;
(5) a heavy chain variable region having the polypeptide sequence of SEQ ID NO:115, and a light chain variable region having the polypeptide sequence of SEQ ID NO:117;
(6) a heavy chain variable region having the polypeptide sequence of SEQ ID NO:115, and a light chain variable region having the polypeptide sequence of SEQ ID NO:118;
(7) a heavy chain variable region having the polypeptide sequence of SEQ ID NO:116, and a light chain variable region having the polypeptide sequence of SEQ ID NO:117;
(8) a heavy chain variable region having the polypeptide sequence of SEQ ID NO:116, and a light chain variable region having the polypeptide sequence of SEQ ID NO:118;
(9) a heavy chain variable region having the polypeptide sequence of SEQ ID NO:119, and a light chain variable region having the polypeptide sequence of SEQ ID NO:121;
(10) a heavy chain variable region having the polypeptide sequence of SEQ ID NO:119, and a light chain variable region having the polypeptide sequence of SEQ ID NO:122;
(11) a heavy chain variable region having the polypeptide sequence of SEQ ID NO:119, and a light chain variable region having the polypeptide sequence of SEQ ID NO:123;
(12) a heavy chain variable region having the polypeptide sequence of SEQ ID NO:120, and a light chain variable region having the polypeptide sequence of SEQ ID NO:121;
(13) a heavy chain variable region having the polypeptide sequence of SEQ ID NO:120, and a light chain variable region having the polypeptide sequence of SEQ ID NO:122;
(14) a heavy chain variable region having the polypeptide sequence of SEQ ID NO:120, and a light chain variable region having the polypeptide sequence of SEQ ID NO:123;
(15) a heavy chain variable region having the polypeptide sequence of SEQ ID NO:124, and a light chain variable region having the polypeptide sequence of SEQ ID NO:126;
(16) a heavy chain variable region having the polypeptide sequence of SEQ ID NO:124, and a light chain variable region having the polypeptide sequence of SEQ ID NO:127;
(17) a heavy chain variable region having the polypeptide sequence of SEQ ID NO:128, and a light chain variable region having the polypeptide sequence of SEQ ID NO:131;
(18) a heavy chain variable region having the polypeptide sequence of SEQ ID NO:128, and a light chain variable region having the polypeptide sequence of SEQ ID NO:132;
(19) a heavy chain variable region having the polypeptide sequence of SEQ ID NO:128, and a light chain variable region having the polypeptide sequence of SEQ ID NO:133;
(20) a heavy chain variable region having the polypeptide sequence of SEQ ID NO:128, and a light chain variable region having the polypeptide sequence of SEQ ID NO:134;
(21) a heavy chain variable region having the polypeptide sequence of SEQ ID NO:129, and a light chain variable region having the polypeptide sequence of SEQ ID NO:131;
(22) a heavy chain variable region having the polypeptide sequence of SEQ ID NO:129, and a light chain variable region having the polypeptide sequence of SEQ ID NO:132;
(23) a heavy chain variable region having the polypeptide sequence of SEQ ID NO:129, and a light chain variable region having the polypeptide sequence of SEQ ID NO:133;
(24) a heavy chain variable region having the polypeptide sequence of SEQ ID NO:129, and a light chain variable region having the polypeptide sequence of SEQ ID NO:134;
(25) a heavy chain variable region having the polypeptide sequence of SEQ ID NO:130, and a light chain variable region having the polypeptide sequence of SEQ ID NO:131;
(26) a heavy chain variable region having the polypeptide sequence of SEQ ID NO:130, and a light chain variable region having the polypeptide sequence of SEQ ID NO:132;
(27) a heavy chain variable region having the polypeptide sequence of SEQ ID NO:130, and a light chain variable region having the polypeptide sequence of SEQ ID NO:133;
(28) a heavy chain variable region having the polypeptide sequence of SEQ ID NO:130, and a light chain variable region having the polypeptide sequence of SEQ ID NO:134;
(29) a heavy chain variable region having the polypeptide sequence of SEQ ID NO:136, and a light chain variable region having the polypeptide sequence of SEQ ID NO:139;
(30) a heavy chain variable region having the polypeptide sequence of SEQ ID NO:136, and a light chain variable region having the polypeptide sequence of SEQ ID NO:140;
(31) a heavy chain variable region having the polypeptide sequence of SEQ ID NO:136, and a light chain variable region having the polypeptide sequence of SEQ ID NO:141;
(32) a heavy chain variable region having the polypeptide sequence of SEQ ID NO:137, and a light chain variable region having the polypeptide sequence of SEQ ID NO:139;
(33) a heavy chain variable region having the polypeptide sequence of SEQ ID NO:137, and a light chain variable region having the polypeptide sequence of SEQ ID NO:140;
(34) a heavy chain variable region having the polypeptide sequence of SEQ ID NO:137, and a light chain variable region having the polypeptide sequence of SEQ ID NO:141;
(35) a heavy chain variable region having the polypeptide sequence of SEQ ID NO:138, and a light chain variable region having the polypeptide sequence of SEQ ID NO:139;
(36) a heavy chain variable region having the polypeptide sequence of SEQ ID NO:138, and a light chain variable region having the polypeptide sequence of SEQ ID NO:140;
(37) a heavy chain variable region having the polypeptide sequence of SEQ ID NO:138, and a light chain variable region having the polypeptide sequence of SEQ ID NO:141;
(38) a heavy chain variable region having the polypeptide sequence of SEQ ID NO:142, and a light chain variable region having the polypeptide sequence of SEQ ID NO:145;
(39) a heavy chain variable region having the polypeptide sequence of SEQ ID NO:142, and a light chain variable region having the polypeptide sequence of SEQ ID NO:146;
(40) a heavy chain variable region having the polypeptide sequence of SEQ ID NO:142, and a light chain variable region having the polypeptide sequence of SEQ ID NO:147;
(41) a heavy chain variable region having the polypeptide sequence of SEQ ID NO:143, and a light chain variable region having the polypeptide sequence of SEQ ID NO:145;
(42) a heavy chain variable region having the polypeptide sequence of SEQ ID NO:143, and a light chain variable region having the polypeptide sequence of SEQ ID NO:146;
(43) a heavy chain variable region having the polypeptide sequence of SEQ ID NO:143, and a light chain variable region having the polypeptide sequence of SEQ ID NO:147;
(44) a heavy chain variable region having the polypeptide sequence of SEQ ID NO:144, and a light chain variable region having the polypeptide sequence of SEQ ID NO:145;
(45) a heavy chain variable region having the polypeptide sequence of SEQ ID NO:144, and a light chain variable region having the polypeptide sequence of SEQ ID NO:146;
(46) a heavy chain variable region having the polypeptide sequence of SEQ ID NO:144, and a light chain variable region having the polypeptide sequence of SEQ ID NO:147;
(47) a heavy chain variable region having the polypeptide sequence of SEQ ID NO:148, and a light chain variable region having the polypeptide sequence of SEQ ID NO:151;
(48) a heavy chain variable region having the polypeptide sequence of SEQ ID NO:150, and a light chain variable region having the polypeptide sequence of SEQ ID NO:153;
(49) a heavy chain variable region having the polypeptide sequence of SEQ ID NO:171, and a light chain variable region having the polypeptide sequence of SEQ ID NO:185;
(50) a heavy chain variable region having the polypeptide sequence of SEQ ID NO:171, and a light chain variable region having the polypeptide sequence of SEQ ID NO:186;
(51) a heavy chain variable region having the polypeptide sequence of SEQ ID NO:171, and a light chain variable region having the polypeptide sequence of SEQ ID NO:187;
(52) a heavy chain variable region having the polypeptide sequence of SEQ ID NO:173, and a light chain variable region having the polypeptide sequence of SEQ ID NO:185;
(53) a heavy chain variable region having the polypeptide sequence of SEQ ID NO:173, and a light chain variable region having the polypeptide sequence of SEQ ID NO:186;
(54) a heavy chain variable region having the polypeptide sequence of SEQ ID NO:173, and a light chain variable region having the polypeptide sequence of SEQ ID NO:187;
(55) a heavy chain variable region having the polypeptide sequence of SEQ ID NO:182, and a light chain variable region having the polypeptide sequence of SEQ ID NO:190;
(56) a heavy chain variable region having the polypeptide sequence of SEQ ID NO:183, and a light chain variable region having the polypeptide sequence of SEQ ID NO:195;
(57) a heavy chain variable region having the polypeptide sequence of SEQ ID NO:183, and a light chain variable region having the polypeptide sequence of SEQ ID NO:196;
(58) a heavy chain variable region having the polypeptide sequence of SEQ ID NO:183, and a light chain variable region having the polypeptide sequence of SEQ ID NO:197;
(59) a heavy chain variable region having the polypeptide sequence of SEQ ID NO:183, and a light chain variable region having the polypeptide sequence of SEQ ID NO:198;
(60) a heavy chain variable region having the polypeptide sequence of SEQ ID NO:184, and a light chain variable region having the polypeptide sequence of SEQ ID NO:195;
(61) a heavy chain variable region having the polypeptide sequence of SEQ ID NO:184, and a light chain variable region having the polypeptide sequence of SEQ ID NO:196;
(62) a heavy chain variable region having the polypeptide sequence of SEQ ID NO:184, and a light chain variable region having the polypeptide sequence of SEQ ID NO:197; or
(63) a heavy chain variable region having the polypeptide sequence of SEQ ID NO:184, and a light chain variable region having the polypeptide sequence of SEQ ID NO:198.

Embodiment 30 is the humanized anti-FOLR1 monoclonal antibody or antigen-binding fragment thereof of embodiment 28 or 29, wherein the monoclonal antibody or antigen-binding fragment thereof is capable of binding FOLR1, inducing effector-mediated tumor cell lysis, mediating the recruitment of conjugated drugs, and/or forming a bispecific antibody with another monoclonal antibody or antigen-binding fragment with a cancer-killing effect.

Embodiment 31 is an isolated nucleic acid encoding the anti-FOLR1 monoclonal antibody or antigen-binding fragment thereof of any one of embodiments 28-30.

Embodiment 32 is a vector comprising the isolated nucleic acid of embodiment 31.

Embodiment 33 is a host cell comprising the vector of embodiment 32.

Embodiment 34 is a pharmaceutical composition, comprising the anti-FOLR1 monoclonal antibody or antigen-binding fragment thereof of any one of embodiments 28-30 and a pharmaceutically acceptable carrier.

Embodiment 35 is a method of targeting FOLR1 on a cancer cell surface in a subject in need thereof, comprising administering to the subject in need thereof a pharmaceutical composition comprising the humanized anti-FOLR1 monoclonal antibody or antigen-binding fragment thereof of any one of embodiments 28-30.

Embodiment 36 is a method of treating cancer in a subject in need thereof, comprising administering to the subject the pharmaceutical composition of embodiment 34.

Embodiment 37 is the method of embodiment 36, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

Embodiment 38 is a method of producing the anti-FOLR1 monoclonal antibody or antigen-binding fragment thereof of any one of embodiments 28-30, comprising culturing a cell comprising a nucleic acid encoding the monoclonal antibody or antigen-binding fragment under conditions to produce the monoclonal antibody or antigen-binding fragment, and recovering the antibody or antigen-binding fragment from the cell or culture.

Embodiment 39 is a method of producing the pharmaceutical composition of embodiment 34, comprising combining the monoclonal antibody or antigen-binding fragment thereof with a pharmaceutically acceptable carrier to obtain the pharmaceutical composition.

### EXAMPLES

### Example 1: Identification of Antigen Binding Domains that Specifically Bind FOLR1

The antigen binding domains that specifically bind FOLR1 are anti-FOLR1 mAbs isolated and sequenced as described in PCT/US2019/021084, filed on Mar. 7, 2019, which is incorporated herein by reference in its entirety.

Sequences of heavy and light chain variable regions for the antigen binding domains that specifically bind FOLR1 are provided in Tables 1 and 2, and the CDR regions for the antigen binding domains that specifically bind FOLR1 are provided in Tables 3-6.

TABLE 1
Sequences of heavy chain variable regions for the antigen binding
domains that specifically bind FOLR1

| | | SEQ |
|---|---|---|
| | | ID |
| Name | VH | NO: |
| F4 | EVQLVESGGGLVKPGGSLKLSCAASGFTFSDYGMHWVRQAPEKGLEWVAFISSGSNTIY | 1 |
| | YADIVKGRFAISRDNAKNTLFLQMASLRSEDTALYYCARLAEWDVAYWGQGTLVTVS | |
| | A | |
| F5 | EVQLVESGGELVKPGGSLKLSCAVSGFTFSNYGMSWVRQTPDKRLEWVATISSGGSYT | 3 |
| | YYPDSVKGRFTISRDNDKNTLYLQMSSLKSEDTAMYYCSTQGSSGYVGYWGQGTTLTV | |
| | SS | |
| F7 | EFQLQQSGPELVKPGASVKISCKASGYSFTDYNMNWVKQSNGKSLEWIGVIDPNYGTT | 5 |
| | NYNQKFVGKATLTVDQSSITAYMQLNSLTAEDSAVYFCAIKGYGNPAAYWGQGTLVT | |
| | VSA | |
| F8 | EVMLVESGGGLVKPGGSLKLSCVASGFTLSTYAMSWVRQTPEKRLEWVATISGGGGDT | 7 |
| | YHLDTVKGRFTISRDNAKNTLYLQMSSLRSEDTALYYCARQSHYGSSYYFDNWGQGTT | |
| | LTVSS | |
| F10 | QVQLQQSGPELVKPGASVKISCKASGYAFSSSWMNWVRQRPGKGLEWIGRIYPGDGYT | 9 |
| | HYNGMFKGKATLTADKSSSTGYMQLSSLTSEDSAVYFCTRHGDFPYWYFDVWGTGTT | |
| | VTVSS | |
| F17 | DVQLVESGGGLVQPGGSRKLSCAASGFTFSDFGMHWIRQAPERGLEWVAYMSYTPGTF | 11 |
| | HYADTVKDRFFISRDNAKNTLFLQMTSLRSDDTAMYYCARVHVGTVDYWGQGTSVTV | |
| | SS | |
| F19 | EVKLDETGGGLVQPGRPMKLSCVASGFTFSDYWMNWVRQSPDKGLEWVAQIGNKFHN | 13 |
| | YETYYSDSVKGRFTISRDDSKSSVYLQMNSLRVEDTGIYYCTKLGRGYYVMDYWGQGT | |
| | SVTVSS | |
| F20 | QVQLQQSGAELVKPGASVQLSCKASGYTFASYYLYWVKQRPGQGLEWIGEINPRSGGT | 15 |
| | NFNEKFKSKATVTVDKSSSTAYMQLSSLTSEDSAVYYCSRSGRLRGFYTMDYWGQGTS | |
| | VTVSS | |

VH: heavy chain variable region

TABLE 2
Sequences of light chain variable regions for the antigen binding
domains that specifically bind FOLR1

| | | SEQ |
|---|---|---|
| | | ID |
| Name | VL | NO: |
| F4 | DIVLTQSPATLSVTPGDRISLSCRASQNINNNLHWYQQKSHESPRLLIKFASQSISGIPSRF | 2 |
| | SGSGSGTDFTLNINGVETEDFGMYFCQQIYSWPQLTFGAGTRLELK | |
| F5 | DIQMTQSPSSLSAFLGGKVTITCKASQDITNFIGWYQHKPGKGPRLLISYTSILESGIPSRF | 4 |
| | SGSGSGRDYSFSISNLEPEDIATYYCLQYYNLWTFGGGTKLEIK | |
| F7 | DIQMTQSPSSLSASLGGKVTITCKASQDINKYLAWYQHEPGKGPRLLIRYTSILESGIPSR | 6 |
| | FSGSGSGRDYSFSISNLEPEDIATYYCLQYYNLWTFGGGTKLEIK | |
| F8 | DIQMTQSPASLSASVGEIVTIICRVSENIDSYLAWYQQKQGKSPQLLVYAATNLADGVP | 8 |
| | SRFSGSGSGSQYSLKINSLQSEDVARYYCQHYYTTPPTFGGGTKLDIK | |
| F10 | DIQMTQSPASLSASVGESVTITCRASENIDSYLAWYQQKQGKSPQLLVYAATNLAVGVP | 10 |
| | SRFSGSGSGTQYTLKINSLQSEDVARYYCQHHYSTPPTFGGGTKLEIK | |
| F17 | DVVLTQSPATLSVTPGDSVSLSCRASQNINNNLHWFQQKSHESPRLLIKYASQSISGIPSR | 12 |
| | FSGSGSGTDFTLSINNVETEDFGIYFCQQSNSWPALTFGTGTKVELK | |
| F19 | DIQMTQTTSSLSASLGDRVTLNCRASQDITNHLNWFQQKPDGTFQLLIYYTSRLHSGVP | 14 |
| | SRFSGSGSGTDYSLTISNLEQEDFATYFCQQDSQHPWTFGGGTKLEIK | |
| F20 | NIVMTQSPKSMSVSVGERVTLSCKAGENVGSYVSWYQQKPEQSPELLIYGASNRYTGV | 16 |
| | PDRFTGSGSATDFTLTISSVQAEDLADYYCGQTYRFLTFGAGTKLELK | |

VL: light chain variable region

TABLE 3
CDR regions 1-3 of heavy chain for the antigen binding
domains that specifically bind FOLR1

| Name | HC CDR1 | NO | HC CDR2 | NO | HC CDR3 | NO |
|---|---|---|---|---|---|---|
| F4 | GFTFSDYG | 17 | ISSGSNTI | 18 | ARLAEWDVAY | 19 |
| F5 | GFTFSNYG | 20 | ISSGGSYT | 21 | STQGSSGYVGY | 22 |
| F7 | GYSFTDYN | 23 | IDPNYGTT | 24 | AIKGYGNPAAY | 25 |
| F8 | GFTLSTYA | 26 | ISGGGGDT | 27 | ARQSHYGSSYYFDN | 28 |
| F10 | GYAFSSSW | 29 | IYPGDGYT | 30 | TRHGDFPYWYFDV | 31 |
| F17 | GFTFSDFG | 32 | MSYTPGTF | 33 | ARVHVGTVDY | 34 |
| F19 | GFTFSDYW | 35 | IGNKFHNYET | 36 | TKLGRGYYVMDY | 37 |
| F20 | GYTFASYY | 38 | INPRSGGT | 39 | SRSGRLRGFYTMDY | 40 |

HC: heavy chain; CDR: complementarity determining region; ID: SEQ ID NO

The HC CDRs for the antigen binding domains that specifically bind FOLR1 were determined utilizing the IMGT method (Lefranc, M.-P. et al., Nucleic Acids Res. 1999; 27:209-212).

TABLE 4
CDR regions 1-3 of light chain for the antigen binding
domains that specifically bind FOLR1

| Name | LC CDR1 | NO | LC CDR2 | NO | LC CDR3 | NO |
|---|---|---|---|---|---|---|
| F4 | QNINNN | 41 | FAS | 42 | QQIYSWPQLT | 43 |
| F5 | QDITNF | 44 | YTS | 45 | LQYYNLWT | 46 |
| F7 | QDINKY | 47 | YTS | 48 | LQYYNLWT | 49 |
| F8 | ENIDSY | 50 | AAT | 51 | QHYYTTPPT | 52 |
| F10 | ENIDSY | 53 | AAT | 54 | QHHYSTPPT | 55 |
| F17 | QNINNN | 56 | YAS | 57 | QQSNSWPALT | 58 |
| F19 | QDITNH | 59 | YTS | 60 | QQDSQHPWT | 61 |
| F20 | ENVGSY | 62 | GAS | 63 | GQTYRFLT | 64 |

LC: light chain; CDR: complementarity determining region; NO: SEQ ID NO

The LC CDRs for the antigen binding domains that specifically bind FOLR1 were determined utilizing the IMGT method (Lefranc, M.-P. et al., Nucleic Acids Res. 1999; 27:209-212).

TABLE 5
CDR regions 1-3 of heavy chain for the antigen binding
domains that specifically bind FOLR1

| Name | HC CDR1 | NO | HC CDR2 | NO | HC CDR3 | NO |
|---|---|---|---|---|---|---|
| F4 | GFTFSDYGMH | 65 | FISSGSNTIYYADIVKG | 66 | ARLAEWDVAY | 67 |
| F5 | GFTFSNYGMS | 68 | TISSGGSYTYYPDSVKG | 69 | STQGSSGYVGY | 70 |
| F7 | GYSFTDYNMN | 71 | VIDPNYGTTNYNQKFVG | 72 | AIKGYGNPAAY | 73 |
| F8 | GFTLSTYAMS | 74 | TISGGGGDTYHLDTVKG | 75 | ARQSHYGSSYYFDN | 76 |
| F10 | GYAFSSSWMN | 77 | RIYPGDGYTHYNGMFKG | 78 | TRHGDFPYWYFDV | 79 |
| F17 | GFTFSDFGMH | 80 | YMSYTPGTFHYADTVKD | 81 | ARVHVGTVDY | 82 |
| F19 | GFTFSDYWMN | 83 | QIGNKFHNYETYYSDSVKG | 84 | TKLGRGYYVMDY | 85 |
| F20 | GYTFASYYLY | 86 | EINPRSGGTNFNEKFKS | 87 | SRSGRLRGFYTMDY | 88 |

HC: heavy chain; CDR: complementarity determining region; NO: SEQ ID NO

The HC CDRs for the antigen binding domains that specifically bind FOLR1 were determined utilizing a combination of IMGT (Lefranc, M.-P. et al., Nucleic Acids Res. 1999; 27:209-212) and Kabat (Elvin A. Kabat et al, Sequences of Proteins of Immunological Interest 5th ed. 1991) methods.

TABLE 6
CDR regions 1-3 of light chain for the antigen binding
domains that specifically bind FOLR1

| Name | LC CDR1 | NO | LC CDR2 | NO | LC CDR3 | NO |
|---|---|---|---|---|---|---|
| F4 | RASQNINNNLH | 89 | FASQSIS | 90 | QQIYSWPQLT | 91 |
| F5 | KASQDITNFIG | 92 | YTSILES | 93 | LQYYNLWT | 94 |
| F7 | KASQDINKYLA | 95 | YTSILES | 96 | LQYYNLWT | 97 |
| F8 | RVSENIDSYLA | 98 | AATNLAD | 99 | QHYYTTPPT | 100 |
| F10 | RASENIDSYLA | 101 | AATNLAV | 102 | QHHYSTPPT | 103 |
| F17 | RASQNINNNLH | 104 | YASQSIS | 105 | QQSNSWPALT | 106 |
| F19 | RASQDITNHLN | 107 | YTSRLHS | 108 | QQDSQHPWT | 109 |
| F20 | KAGENVGSYVS | 110 | GASNRYT | 111 | GQTYRFLT | 112 |

LC: light chain; CDR: complementarity determining region; NO: SEQ ID NO

The LC CDRs for the antigen binding domains that specifically bind FOLR1 were determined utilizing a combination of IMGT (Lefranc, M.-P. et al., Nucleic Acids Res. 1999; 27:209-212) and Kabat (Elvin A. Kabat et al, Sequences of Proteins of Immunological Interest 5th ed. 1991) methods.

### Example 2: Humanization of Mouse Anti-FOLR1 mAbs

The mouse anti-FOLR1 mAbs were humanized to reduce the potential of immunogenicity when used in human patients as described in PCT/US2019/021084, filed on Mar. 7, 2019, which is incorporated herein by reference in its entirety. The sequences of the humanized VH and VL regions are shown in Table 7. The humanized VH and VL were named as follows: F5-H1 refers to the H1 sequence of humanized VH for mouse mAb F5; F5-L1 refers to the L1 sequence of humanized VL for mouse mAb F5. All the other humanized VH and VL regions adopt the same naming rule.

TABLE 7
Sequences of heavy chain and light chain variable regions of humanized
antigen binding domains that specifically bind FOLR1

| | | SEQ |
|---|---|---|
| | | ID |
| VH/VL | SEQUENCE | NO: |
| F5-H1 | EVQLLESGGGLVQPGGSLRLSCAVSGFTFSNYGMSWVRQAPGKGLEWVATISSGGSYTY | 113 |
| | YPDSVKGRFTISRDNSKNTLYLQMNSLRAEDTAVYYCSTQGSSGYVGYWGQGTLVTVSS | |
| F5-H2 | EVQLVESGGGLVQPGGSLRLSCAVSGFTFSNYGMSWVRQAPGKGLEWVATISSGGSYTY | 114 |
| | YPDSVKGRFTISRDNSKNTLYLQMNSLRAEDTAVYYCSTQGSSGYVGYWGQGTLVTVSS | |
| F5-H3 | EVQLVESGGGLVQPGGSLRLSCAVSGFTFSNYGMSWVRQAPGKGLEWVATISSGGSYTY | 115 |
| | YPDSVKGRFTISRDNSKNTLYLQMSSLRAEDTAVYYCSTQGSSGYVGYWGQGTLVTVSS | |
| F5-H4 | EVQLVESGGGLVQPGGSLRLSCAVSGFTFSNYGMSWVRQAPGKGLEWVATISSGGSYTY | 116 |
| | YPDSVKGRFTISRDNDKNTLYLQMSSLRAEDTAVYYCSTQGSSGYVGYWGQGTLVTVSS | |
| F5-L1 | DIQMTQSPSSVSASVGDRVTITCKASQDITNFIGWYQHKPGKAPKLLISYTSILESGVPSR | 117 |
| | FSGSGSGRDYTLTISSLQPEDFATYYCLQYYNLWTFGGGTKVEIK | |
| F5-L2 | DIQMTQSPSSVSASVGDRVTITCKASQDITNFIGWYQHKPGKAPKLLISYTSILESGVPSR | 118 |
| | FSGSGSGTDYTLTISSLQPEDFATYYCLQYYNLWTFGGGTKVEIK | |
| F10- | EVQLVQSGAEVKKPGSSVKVSCKASGYAFSSSWMNWVRQAPGQGLEWIGRIYPGDGYT | 119 |
| H1 | HYNGMFKGRASLTADKSTSTGYMELSSLRSEDTAVFFCTRHGDFPYWYFDVWGRGTLV | |
| | TVSP | |
| F10- | EVQLVQSGAEVKKPGSSVKVSCKASGYAFSSSWMNWVRQAPGQGLEWIGRIYPGDGYT | 120 |
| H2 | HYNGMFKGRASLTADKSTSTGYMELSSLRSEDTAVFFCTRHGDFPYWYFDVWGRGTLV | |
| | TVSS | |
| F10- | DIQMTQSPSTLSASVGDRVTITCRASENIDSYLAWYQQKPGRAPKLLVYAATNLAVGVPS | 121 |
| L1 | RFSGSGSGTEYTLTISSLQSDDFATYYCQHHYSTPPTFGQGTKLEIK | |
| F10- | DIQMTQSPSTLSASVGDRVTITCRASENIDSYLAWYQQKPGRAPKLLVYAATNLAVGVPS | 122 |
| L2 | RFSGSGSGTEYTLTISSLQPDDFATYYCQHHYSTPPTFGQGTKLEIK | |
| F10- | DIQMTQSPSTLSASVGDRVTITCRASENIDSYLAWYQQKPGRAPKLLVYAATNLAVGVPS | 123 |
| L3 | RFSGSGSGTEYTLTISSLQSEDFATYYCQHHYSTPPTFGQGTKLEIK | |
| F17- | EVQLVETGGGLIQPGGSLRLSCAASGFTFSDFGMHWIRQAPGKGLEWVAYMSYTPGTFH | 124 |
| H1 | YADTVKDRFTISRDNSKNTLYLQMNSLRAEDTAVYYCARVHVGTVDYWGQGTLVTVSS | |
| F17- | QVQLVESGGGVVQPGRSLRLSCAASGFTFSDFGMHWIRQAPGKGLEWVAYMSYTPGTF | 125 |
| H2 | HYADTVKDRFTISRDNSKNTLYLQMNSLRAEDTAVYYCARVHVGTVDYWGQGTLVTVSS | |
| F17- | EVVLTQSPATLSLSPGERATLSCRASQNINNNLHWFQQKPGQAPRLLIKYASQSISGIPARF | 126 |
| L1 | SGSGSGTDFTLTISSLEPEDFAVYFCQQSNSWPALTFGQGTKVEIK | |
| F17- | EVVLTQSPATLSLSPGERATLSCRASQNINNNLHWFQQKPGQAPRLLIKYASQSISGIPARF | 127 |
| L2 | SGSGSGTDFTLTISSLETEDFAVYFCQQSNSWPALTFGQGTKVEIK | |
| F20- | QVQLVQSGAEVKKPGSSVKVSCKASGYTFASYYLYWVRQAPGQGLEWIGEINPRSGGTN | 128 |
| H1 | FNEKFKSRATVTVDKSTSTAYMELSSLRSEDTAVYYCSRSGRLRGFYTMDYWGQGTLVT | |
| | VSS | |
| F20- | QVQLVQSGAEVKKPGSSVKVSCKASGYTFASYYLYWVRQAPGQGLEWIGEINPRSGGTN | 129 |
| H2 | FNEKFKSRATVTVDASTSTAYMELSSLRSEDTAVYYCSRSGRLRGFYTMDYWGQGTLVT | |
| | VSS | |
| F20- | QVQLVQSGAEVKKPGSSVKVSCKASGYTFASYYLYWVRQAPGQGLEWIGEINPRSGGTN | 130 |
| H3 | FNEKFKSKATVTVDKSTNTAYMELSSLRSEDTAVYYCSRSGRLRGFYTMDYWGQGTLV | |
| | TVSS | |
| F20- | DIVMTQSPDSLAVSLGERATINCKAGENVGSYVSWYQQKPGQPPKLLIYGASNRYTGVP | 131 |
| L1 | DRFSGSGSATDFTLTISSLQAEDVAVYYCGQTYRFLTFGQGTKVEIK | |
| F20- | DIVMTQSPDSLAVSLGERATINCKAGENVGSYVSWYQQKPGQSPKLLIYGASNRYTGVP | 132 |
| L2 | DRFSGSGSATDFTLTISSLQAEDVAVYYCGQTYRFLTFGQGTKVEIK | |
| F20- | DIQMTQSPSTLSASVGDRVTITCKAGENVGSYVSWYQQKPGKAPKLLIYGASNRYTGVP | 133 |
| L3 | ARFSGSGSATEFTLTISSLQPDDFATYYCGQTYRFLTFGQGTKVEVK | |
| F20- | DIQMTQSPSTLSASVGDRVTITCKAGENVGSYVSWYQQKPGKAPKLLIYGASNRYTGVP | 134 |
| L4 | ARFSGSGSATEFTLTISSLQPEDFATYYCGQTYRFLTFGQGTKVEVK | |
| F4-H1 | QVQLVQSGAEVRKPGASVKVSCKASGFTFSDYGMHWVRQAPGQGLEWVAFISSGSNTI | 136 |
| | YYADIVKGRFTITRNNSTSTLYMELSSLRSEDTAIYYCARLAEWDVAYWGAGTLVTVSS | |
| F4-H2 | QVQLVQSGAEVKKPGASVKVSCKASGFTFSDYGMHWVRQAPGQGLEWVAFISSGSNTI | 137 |
| | YYADIVKGRVTITRNNSTSTLYMELSSLRSEDTAIYYCARLAEWDVAYWGAGTLVTVSS | |
| F4-H3 | EVQLVESGGGLVQPGRSLRLSCAASGFTFSDYGMHWVRQAPGKGLEWVAGISSGSNTIG | 138 |
| | YADS\VQGRFTISRDNGKNSLYLQMNSLRAEDTALYYCARLAEWDVAYWGQGTMVTVSS | |
| F4-L1 | DIQLTQSPSTLSASIGDRVTITCRASQNINNNLHWYQQKPGKAPKLLIKFASQSISGAPSRF | 139 |
| | SGSGSGTDFTLTISSLQPDDFATYFCQQIYSWPQLTFGGGTKLEIK | |
| F4-L2 | DIQLTQSPSSLSASVGDRVTITCRASQNINNNLHWYQQKPGKAPKLLIKFASQSISGVPSRF | 140 |
| | SGSGSGTDFTLTISSLQPDDFATYFCQQIYSWPQLTFGGGTKLEIK | |
| F4-L3 | EIVLTQSPGTLSLSPGERATLSCRASQNINNNLHWYQQKPGQAPRLLIKFASTRATGIPARF | 141 |
| | SGSGSGTDFTLTISRLEPEDLAVYFCQQIYSWPQLTFGQGTKVEIK | |
| F7-H1 | QFQLVQSGAEVKKPGASVKVSCKASGYSFTDYNMHWVRQAPGQGLEWIGWIDPNYGTT | 142 |
| | NYAQKFQGRATLTVDQSISTAYMELSRLRSDDTAVYFCAIKGYGNPAAYWGQGTLVTVSS | |
| F7-H2 | EFQLVESGAEVKKPGSSVKVSCKASGYSFTDYNFTWVRQAPGQGLEWIGRIDPNYGTTH | 143 |
| | YAPHLQGRATLTVDQSTSTAYLELRNLRSDDTAVYFCAIKGYGNPAAYWGQGTLVTVSS | |
| F7-H3 | QVQLVQSGAEVKKPGASVKVSCKASGYSFTDYNFTWVRQAPGQGLEWIGRIDPNYGTT | 144 |
| | HYAPHLQGRATLTVDQSTSTAYLELRSLRSDDTAVYFCAIKGYGNPAAYWGQGTLVTVSS | |
| F7-L1 | DIVMTQSPDSLAVSLGERATINCKASQDINKYLAWYQHKPGQPPKLLIRYTSTRESGVPD | 145 |
| | RFSGSGSGRDYTLTISSLQAEDVAVYYCLQYYNLWTFGGGTKVEIK | |
| F7-L2 | DIVMTQSPATLSVSPGERATLSCRASQDINKYLAWYQHKPGQAPRLLIRYTSTRATGVPA | 146 |
| | RFSGSGSGREYTLTISSLQSEDFAVYYCLQYYNLWTFGQGTRLEIK | |
| F7-L3 | DIVMTQSPATLSVSPGERATLSCRASQDINKYLAWYQHKPGQAPRLLIRYTSTRATGIPAR | 147 |
| | FSGSGSGTEFTLTISSLQSEDFAVYYCLQYYNLWTFGQGTRLEIK | |
| F8-H1 | LVNLVESGGGVVQPGRSLRLSCAASGFTLSTYAMHWVRQAPGKGLEWVAVISGGGGDT | 148 |
| | YYADSVKGRFTISRDNSKNTLYLQMNSLRAEDTAVFYCARQSHYGSSYYFDNWGQGTL | |
| | VTVSS | |
| F8-H2 | QVQLVESGGGVVQPGRSLRLSCAASGFTLSTYAMNWVRQAPAKGLEWVAIISGGGGDT | 149 |
| | YYADSVKGRFTISRDNSKNTLYLQMNGLRAEDTAVYYCARQSHYGSSYYFDNWGQGTL | |
| | VTVSS | |
| F8-H3 | QVQLVESGGGVVQPGRSLRLSCAASGFTLSTYAMSWVRQAPAKGLEWVAIISGGGGDT | 150 |
| | YYADSVKGRFTISRDNSKNTLYLQMNGLRAEDTAVYYCARQSHYGSSYYFDNWGQGTL | |
| | VTVSS | |
| F8-L1 | EIQMTQSPATLSLSPGERATLSCRVSENIDSYLAWYQQKPGQAPRLLVYAATNRATGIPA | 151 |
| | RFSGSGSGSDYTLTISSLEPEDFAVYYCQHYYTTPPTFGQGTKVEIK | |
| F8-L2 | EIVMTQSPDFQSVTPKEKVTITCRVSENIDSYLAWYQQKPDQSPKLLVYAATQSFSGVPSR | 152 |
| | FSGSGSGSDYTLTINSLEAEDAAAYYCQHYYTTPPTFGPGTKVDIK | |
| F8-L3 | EIVLTQSPDFQSVTPKEKVTITCRVSENIDSYLAWYQQKPDQSPKLLVYAATQSFSGVPSR | 153 |
| | FSGSGSGTDFTLTINSLEAEDAAAYYCQHYYTTPPTFGPGTKVDIK | |
| F19- | QVQLQESGPGLVRPSQTLSLTCTASGFTFSDVRQPPGRGLEWVAFIGNKFHNY | 154 |
| H1 | ETEYNPSVKGRFTILRDDSKNQVSLRLSSVTAADTAVYYCTKLGRGYYVMDYWGQGS | |
| | LVTVSS | |
| F19- | QVQLQESGPGLVKPSQTLSLTCTASGFTFSDVRQPPGKGLEWVAFIGNKFHNY | 155 |
| H2 | ETEYNPSVKGRFTILRDDSKNQVSLKLSSVTAADTAVYYCTKLGRGYYVMDYWGQGS | |
| | LVTVSS | |
| F19- | QVQLVESGGGVVQPGRSLRLSCAASGFTFSDYWMNWVRQAPGKGLEWVAIIGNKFHNY | 156 |
| H3 | ETYYADSVKGRFTISRDDSKNTVYLQMNGLRAEDTAVYYCTKLGRGYYVMDYWGQGT | |
| | LVTVSS | |
| F19- | DIQMTQSPSSLSASVGDRVTITCKASQDITNHLNWFQQKPGKAFKLLIYYTSNLQTGVPSR | 157 |
| L1 | FSGSGSGTDYTFTISSLQPEDIATYFCQQDSQHPWTFGQGTKVEIK | |
| F19- | EIVMTQSPDFQSVTPKEKVTITCRASQDITNHLNWFQQKPDQSFKLLIYYTSQSFSGVPSR | 158 |
| L2 | FSGSGSGTDYTLTINSLEAEDAAAYFCQQDSQHPWTFGPGTKVDIK | |
| F8-H4 | QVQLVQSGGGLVKPGGSLRLSCAASGFTLSTYAMSWVRQAPGKGLEWVAAISGGGGD | 171 |
| | TYYADSVKGRFTISRDNSKNTLYLQMSSLRAEDTAVYYCARQSHYGSSYYFDNWGQGT | |
| | MVTVSS | |
| F8-H5 | QVQLVQSGGGLVKPGGSLRLSCAASGFTLSTYAMSWVRQAPGKGLEWVAAISGGGGD | 172 |
| | TYHLDSVKGRFTISRDNSKNTLYLQMSSLRAEDTAVYYCARQSHYGSSYYFDNWGQGT | |
| | MVTVSS | |
| F8-H6 | EVQLVVSGGGLIQPGGSLRLSCAASGFTLSTYAMTWVRQAPGKGLEWVAVISGGGGD | 173 |
| | TYYADSVVGRFTISRDNSKNTLYLQMNSLRAEDTAVYYCARQSHYGSSYYFDNWGQ | |
| | GTLVTVSS | |
| F19- | EVVLVESGGGLVQPGGSLRLSCAASGFTFSDYWISWVRQAPGKGLEWVAHIGNKFHN | 174 |
| H4 | YETDYADSVKGRFTISRDDSKNTVYLQMNSLRAEDTAVYYCTKLGRGYYVMDYWG | |
| | QGTLVTVSS | |
| F19- | EVQLVESGGGLVQPGGSLRLSCAASGFTFSDYWISWVRQAPGKGLEWVAHIGNKFHN | 175 |
| H5 | YETDYADSVKGRFTISKDDSKNTVYLQMNSLRAEDTAVYYCTKLGRGYYVMDYWG | |
| | QGTLVTVSS | |
| F19- | QVQLVESGGGVVQPGRSLRLSCAASGFTFSDYWMHWVRQAPGKGLEWVAVIGNKFH | 176 |
| H6 | NYETYYADSVKGRFTISRDDSKNTVYLQMNSLRAEDTAVYYCTKLGRGYYVMDYWG | |
| | QGTTVTVSS | |
| F19- | EVQLVESGGGLVQPGGSLRLSCAASGFTFSDVRQAPGKGLEWVAHIGNKFHN | 177 |
| H7 | YETDYADSVKGRFTISKDDSKNTVYLQMNSLRAEDTAVYYCTKLGRGYYVMDYWGQ | |
| | GTLVTVSS | |
| F19- | QVQLVESGGGVVQPGRSLRLSCAASGFTFSDYWMHWVRQAPGKGLEWVAVIGNKFHN | 178 |
| H8 | YETYYTDSVKGRFTISRDDSKNTVYLQMNTLRAEDTAVYYCTKLGRGYYVMDYWGKG | |
| | TTVTVSS | |
| F19- | QVQLVESGGGVVQPGRSLRLSCAASGFTFSDYWMHWVRQAPGKGLEWVAVIGNKFHN | 179 |
| H9 | YETYYTDSVKGRFTISRDNSKNTLYLQMNSLRAEDTAVYYCTKLGRGYYVMDYWGKG | |
| | TTVTVSS | |
| F19- | QVVLVESGGGLVQPGGSLRLSCAASGFTFSDYWMHWVRQAPGKGLEWVASIGNKFHN | 180 |
| H10 | YETYYADSVKGRFTISRDDSKNTVYLQMNSLFAEDTAVYYCTKLGRGYYVMDYWGQG | |
| | TLVTVSS | |
| F19- | QVQLVESGGGVVQPGGSLRLSCAASGFTFSDYWMHWVRQAPGKGLEWVAFIGNKFH | 181 |
| H11 | NYETYYADSVKGRFTISRDNSKNTLYLQMNSLRAEDTAVYYCTKLGRGYYVMDYWG | |
| | QGTLVTVSS | |
| F19- | EVQLVESGGGLVQPGGSLRLSCVASGFTFSDVRQAPGKGLEWVAQIGNKFH | 182 |
| H12 | NYETYYSDSVKGRFTISKDDSKNTVYLQMNSLRAEDTAVYYCTKLGRGYYVMDYWG | |
| | QGTLVTVSS | |
| F5-H5 | QVELVESGGGVVQPGRSLRLDCKVSGFTFSNYGMHWVRQAPGKGLEWVAVISSGGS | 183 |
| | YTYYADSVKGRFTISRDNSKNTLFLQMNSLRAEDTAVYYCSTQGSSGYVGYWGQGT | |
| | LVTVSS | |
| F5-H6 | QVQLVESGGGVVQPGRSLRLSCKVSGFTFSNYGMHWVRQAPGKGLEWVAVISSGGS | 184 |
| | YTYYADSVKGRFTISRDNSKNTLFLQMNSLRAEDTAVYYCSTQGSSGYVGYWGQG | |
| | TLVTVSS | |
| F8-L4 | DIQMTQSPSSLSASVGDRVTITCRVSENIDSYLAWYQQKPGKAPKLLVYAATSLQSGV | 185 |
| | PSRFSGSGSGSDYTLTISSLQPEDFAVYYCQHYYTTPPTFGDGTKVEIK | |
| F8-L5 | DIQMTQSPSSLSASVGDRVTITCRVSENIDSYLAWYQQKPGKAPKLLVYAATNLASG | 186 |
| | VPSRFSGSGSGSDYTLTISSLQPEDFAVYYCQHYYFIPPTFGDGTKVEIK | |
| F8-L6 | DIQMTQSPSTLSASVGDRVTITCRVSENIDSYLAWYQQKPGKAPKLLVYAATSLESG | 187 |
| | VPSRFSGSGSGSEYTLTISSLQPDDFATYYCQHYYTTPPTFGQGTKVDIK | |
| F19- | DIQMTQSPSSLSASVGDRVTITCRASQDITNHLNWFQQKPGKAFKLLIYYTSSRASG | 188 |
| L3 | VPSRFSGSGSGTDYTLTISSLQPEDFATYFCQQDSQHPWTFGQGTKVEIK | |
| F19- | DIQMTQSPSSLSASVGDRVTITCRASQDITNHLNWFQQKPGKAFKLLIYYTSSLQSG | 189 |
| L4 | VPSRFSGSGSGTDYTLTISSLQPEDFATYFCQQDSQHPWTFGPGTKVEIK | |
| F19- | DIQMTQSPSSLSASVGDRVTITCRASQDITNHLNWFQQKPGKAFKLLIYYTSRLHSG | 190 |
| L5 | VPSRFSGSGSGTDYTLTISSLQPEDFATYFCQQDSQHPWTFGGGTKVEIK | |
| F19- | DIQMTQSPSSLSASVGDRVTITCRASQDITNHLNWFQQKPGKAFKLLIYYTSSLQSG | 191 |
| L8 | VPSRFSGSGSGTDYTLTISSLQPEDFATYFCQQDSQHPWTFGGGTKVEIK | |
| F19- | DIQMTQSPSSLSASVGDRVTITCRASQDITNHLNWYQQKPGKAPKLLIYYTSSLQSG | 192 |
| L9 | VPSRFSGSGSGTDFTLTISSLQPEDFATYYCQQDSQHPWTFGGGTKVEIK | |
| F19- | EIVMTQSPATLSLSPGERATLSCRASQDITNHLNWFQQKPGQAFRLLIYYTSSRATG | 193 |
| L10 | VPARFSGSGSGTDFTLTISSLEPEDFAVYFCQQDSQHPWTFGQGTKVEIK | |
| F19- | EIVLTQSPATLSLSPGERATLSCRASQDITNHLAWYQQKPGQAPRLLIYYTSNRATG | 194 |
| L11 | IPARFSGSGSGTDFTLTISSLEPEDFAVYYCQQDSQHPWTFGQGTKVEIK | |
| F5-L3 | AIQMTQSPSSLSASVGDRVTITCRASQDITNFLGWYQHKPGEAPKLLISYTSVLQSG | 195 |
| | VPSRFSGSGSGRDYTLTISSLQPEDFATYYCLQYYNLWTFGQGTKVEIK | |
| F5-L4 | EIVMTQSPDFQSVTPKEKVTITCRASQDITNFIGWYQHKPDQSPKLLISYTSQSFSGV | 196 |
| | PSRFSGSGSGRDYTLTINSLEAEDAAAYYCLQYYNLWTFGPGTKVDIK | |
| F5-L5 | EIVMTQSPATLSLSPGERATLSCRASQDITNFLGWYQHKPGQAPRLLISYTSNRATG | 197 |
| | IPARFSGSGSGRDYTLTISSLEPEDFAVYYCLQYYNLWTFGQGTKVEIK | |
| F5-L6 | EIVMTQSPATLSLSPGERATLSCRASQDITNFLGWYQQKPGQAPRLLISYTSIRATGI | 198 |
| | PARFSGSGSGTDYTLTISSLEPEDFAVYYCLQYYNLWTFGQGTKVEIK | |

The humanized VH and VL regions were fused to the constant regions of human IgG1 heavy chain and kappa light chain, respectively. The humanized mAbs were named as follows: F5-H1L1 refers to the mAb with the F5-H1 heavy chain variable region and the F5-L1 light chain variable region; all the other humanized mAbs adopt the same naming rule.

### Example 3: Conversion of Humanized mAbs to Single Chain Variable Fragments (scFvs)

The humanized mAbs were converted to scFvs, each of which consists of one VH and one VL with a (G₄S)ₙ linker in between (where “n” represents the number of the G₄S repeats). Either the VH or the VL region was placed at the N-terminus of the fusion protein to identify the most effective scFv designs. The sequences of the designed scFvs are shown in Table 8. The scFvs were named as following: F5-H2(G₄S)₃L2 refers to the scFv with F5-H2 heavy chain variable region, the (G₄S)₃ linker and F5-L2 light chain variable region; all the other scFvs adopt the same naming rule.

TABLE 9
Sequences of humanized scFvs that specifically bind FOLR1

| | | SEQ |
|---|---|---|
| | | ID |
| Name | SEQUENCE | NO: |
| F5- | EVQLVESGGGLVQPGGSLRLSCAVSGFTFSNYGMSWVRQAPGKGLEWVATISSG | 159 |
| H2(G₄S)₃L2 | GSYTYYPDSVKGRFTISRDNSKNTLYLQMNSLRAEDTAVYYCSTQGSSGYVGY | |
| | WGQGTLVTVSSGGGGSGGGGSGGGGSDIQMTQSPSSVSASVGDRVTITCKASQD | |
| | ITNFIGWYQHKPGKAPKLLISYTSILESGVPSRFSGSGSGTDYTLTISSLQPEDFAT | |
| | YYCLQYYNLWTFGGGTKVEIK | |
| F5- | EVQLVESGGGLVQPGGSLRLSCAVSGFTFSNYGMSWVRQAPGKGLEWVATISSG | 160 |
| H2(G₄S)₄L2 | GSYTYYPDSVKGRFTISRDNSKNTLYLQMNSLRAEDTAVYYCSTQGSSGYVGY | |
| | WGQGTLVTVSSGGGGSGGGGSGGGGSGGGGSDIQMTQSPSSVSASVGDRVTITC | |
| | KASQDITNFIGWYQHKPGKAPKLLISYTSILESGVPSRFSGSGSGTDYTLTISSLQP | |
| | EDFATYYCLQYYNLWTFGGGTKVEIK | |
| F5- | DIQMTQSPSSVSASVGDRVTITCKASQDITNFIGWYQHKPGKAPKLLISYTSILESG | 161 |
| L2(G₄S)₃H2 | VPSRFSGSGSGTDYTLTISSLQPEDFATYYCLQYYNLWTFGGGTKVEIKGGGGSG | |
| | GGGSGGGGSEVQLVESGGGLVQPGGSLRLSCAVSGFTFSNYGMSWVRQAPGKG | |
| | LEWVATISSGGSYTYYPDSVKGRFTISRDNSKNTLYLQMNSLRAEDTAVYYCST | |
| | QGSSGYVGYWGQGTLVTVSS | |
| F10- | EVQLVQSGAEVKKPGSSVKVSCKASGYAFSSSWMNWVRQAPGQGLEWIGRIYP | 162 |
| H1(G₄S)₃L2 | GDGYTHYNGMFKGRASLTADKSTSTGYMELSSLRSEDTAVFFCTRHGDFPYWY | |
| | FDVWGRGTLVTVSPGGGGSGGGGSGGGGSDIQMTQSPSTLSASVGDRVTITCRA | |
| | SENIDSYLAWYQQKPGRAPKLLVYAATNLAVGVPSRFSGSGSGTEYTLTISSLQP | |
| | DDFATYYCQHHYSTPPTFGQGTKLEIK | |
| F10- | EVQLVQSGAEVKKPGSSVKVSCKASGYAFSSSWMNWVRQAPGQGLEWIGRIYP | 163 |
| H1(G₄S)₄L2 | GDGYTHYNGMFKGRASLTADKSTSTGYMELSSLRSEDTAVFFCTRHGDFPYWY | |
| | FDVWGRGTLVTVSPGGGGSGGGGSGGGGSGGGGSDIQMTQSPSTLSASVGDRV | |
| | TITCRASENIDSYLAWYQQKPGRAPKLLVYAATNLAVGVPSRFSGSGSGTEYTLT | |
| | ISSLQPDDFATYYCQHHYSTPPTFGQGTKLEIK | |
| F10- | DIQMTQSPSTLSASVGDRVTITCRASENIDSYLAWYQQKPGRAPKLLVYAATNL | 164 |
| L2(G₄S)₃H1 | AVGVPSRFSGSGSGTEYTLTISSLQPDDFATYYCQHHYSTPPTFGQGTKLEIKGGG | |
| | GSGGGGSGGGGSEVQLVQSGAEVKKPGSSVKVSCKASGYAFSSSWMNWVRQA | |
| | PGQGLEWIGRIYPGDGYTHYNGMFKGRASLTADKSTSTGYMELSSLRSEDTAVF | |
| | FCTRHGDFPYWYFDVWGRGTLVTVSP | |
| F17- | EVQLVETGGGLIQPGGSLRLSCAASGFTFSDFGMHWIRQAPGKGLEWVAYMSYT | 165 |
| H1(G₄S)₃L2 | PGTFHYADTVKDRFTISRDNSKNTLYLQMNSLRAEDTAVYYCARVHVGTVDYW | |
| | GQGTLVTVSSGGGGSGGGGSGGGGSEVVLTQSPATLSLSPGERATLSCRASQNIN | |
| | NNLHWFQQKPGQAPRLLIKYASQSISGIPARFSGSGSGTDFTLTISSLETEDFAVYF | |
| | CQQSNSWPALTFGQGTKVEIK | |
| F17- | EVQLVETGGGLIQPGGSLRLSCAASGFTFSDFGMHWIRQAPGKGLEWVAYMSYT | 166 |
| H1(G₄S)₄L2 | PGTFHYADTVKDRFTISRDNSKNTLYLQMNSLRAEDTAVYYCARVHVGTVDYW | |
| | GQGTLVTVSSGGGGSGGGGSGGGGSGGGGSEVVLTQSPATLSLSPGERATLSCR | |
| | ASQNINNNLHWFQQKPGQAPRLLIKYASQSISGIPARFSGSGSGTDFTLTISSLETE | |
| | DFAVYFCQQSNSWPALTFGQGTKVEIK | |
| F17- | EVVLTQSPATLSLSPGERATLSCRASQNINNNLHWFQQKPGQAPRLLIKYASQSIS | 167 |
| L2(G₄S)₃H1 | GIPARFSGSGSGTDFTLTISSLETEDFAVYFCQQSNSWPALTFGQGTKVEIKGGGG | |
| | SGGGGSGGGGSEVQLVETGGGLIQPGGSLRLSCAASGFTFSDFGMHWIRQAPGK | |
| | GLEWVAYMSYTPGTFHYADTVKDRFTISRDNSKNTLYLQMNSLRAEDTAVYYC | |
| | ARVHVGTVDYWGQGTLVTVSS | |
| F20- | QVQLVQSGAEVKKPGSSVKVSCKASGYTFASYYLYWVRQAPGQGLEWIGEINP | 168 |
| H1(G₄S)₃L3 | RSGGTNFNEKFKSRATVTVDKSTSTAYMELSSLRSEDTAVYYCSRSGRLRGFYT | |
| | MDYWGQGTLVTVSSGGGGSGGGGSGGGGSDIQMTQSPSTLSASVGDRVTITCK | |
| | AGENVGSYVSWYQQKPGKAPKLLIYGASNRYTGVPARFSGSGSATEFTLTISSLQ | |
| | PDDFATYYCGQTYRFLTFGQGTKVEVK | |
| F20- | QVQLVQSGAEVKKPGSSVKVSCKASGYTFASYYLYWVRQAPGQGLEWIGEINP | 169 |
| H1(G₄S)₄L3 | RSGGTNFNEKFKSRATVTVDKSTSTAYMELSSLRSEDTAVYYCSRSGRLRGFYT | |
| | MDYWGQGTLVTVSSGGGGSGGGGSGGGGSGGGGSDIQMTQSPSTLSASVGDRV | |
| | TITCKAGENVGSYVSWYQQKPGKAPKWYGASNRYTGVPARFSGSGSATEFTLT | |
| | ISSLQPDDFATYYCGQTYRFLTFGQGTKVEVK | |
| F20- | DIQMTQSPSTLSASVGDRVTITCKAGENVGSYVSWYQQKPGKAPKLLIYGASNR | 170 |
| L3(G₄S)₃H1 | YTGVPARFSGSGSATEFTLTISSLQPDDFATYYCGQTYRFLTFGQGTKVEVKGGG | |
| | GSGGGGSGGGGSQVQLVQSGAEVKKPGSSVKVSCKASGYTFASYYLYWVRQAP | |
| | GQGLEWIGEINPRSGGTNFNEKFKSRATVTVDKSTSTAYMELSSLRSEDTAVYYC | |
| | SRSGRLRGFYTMDYWGQGTLVTVSS | |

### Example 4: ELISA Binding Analysis of Humanized mAbs and scFvs

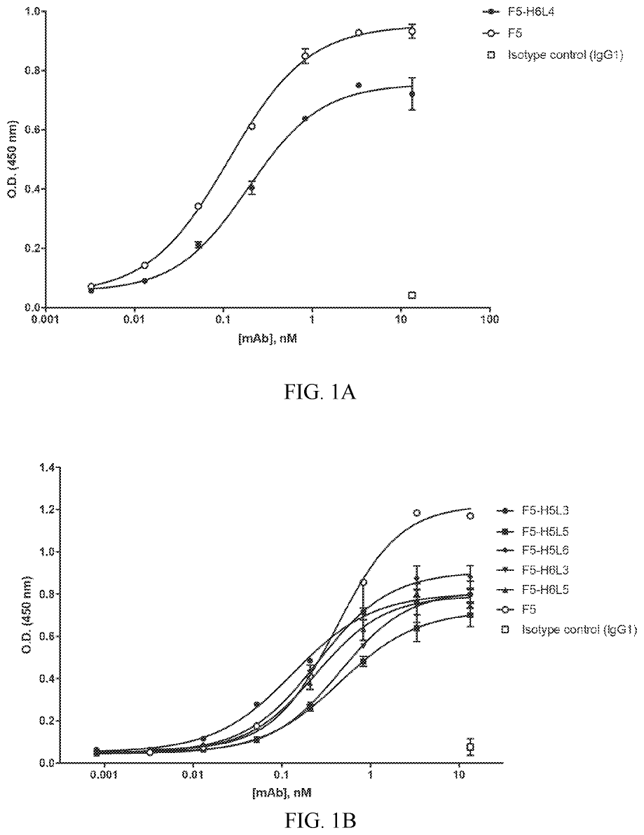

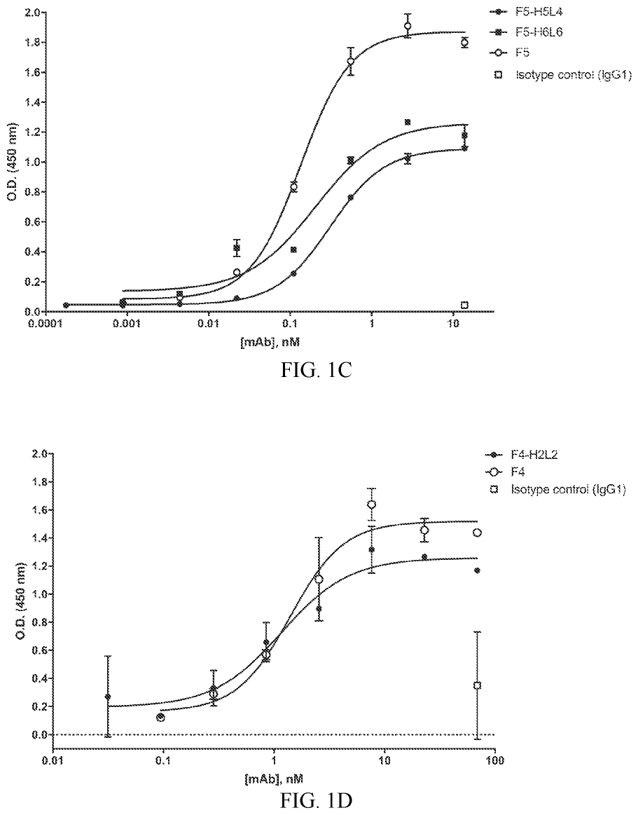

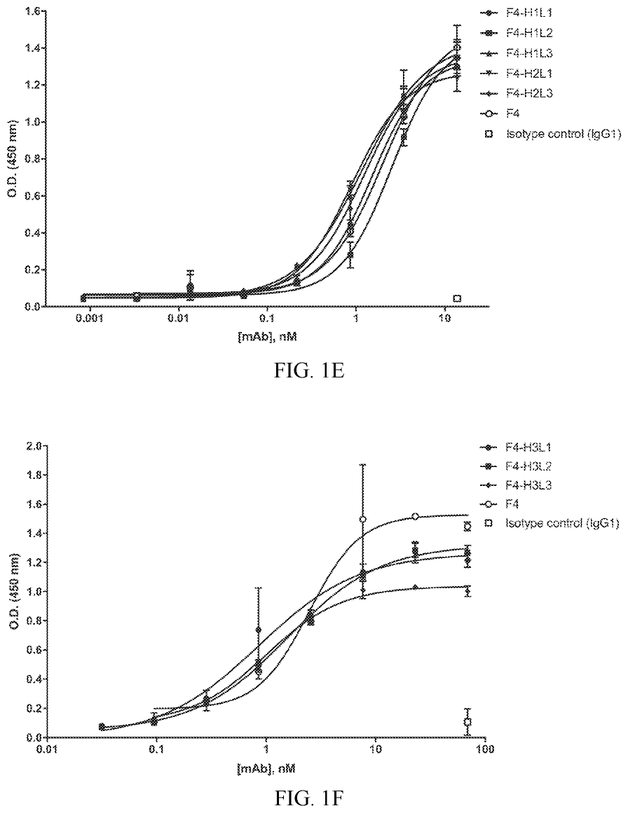

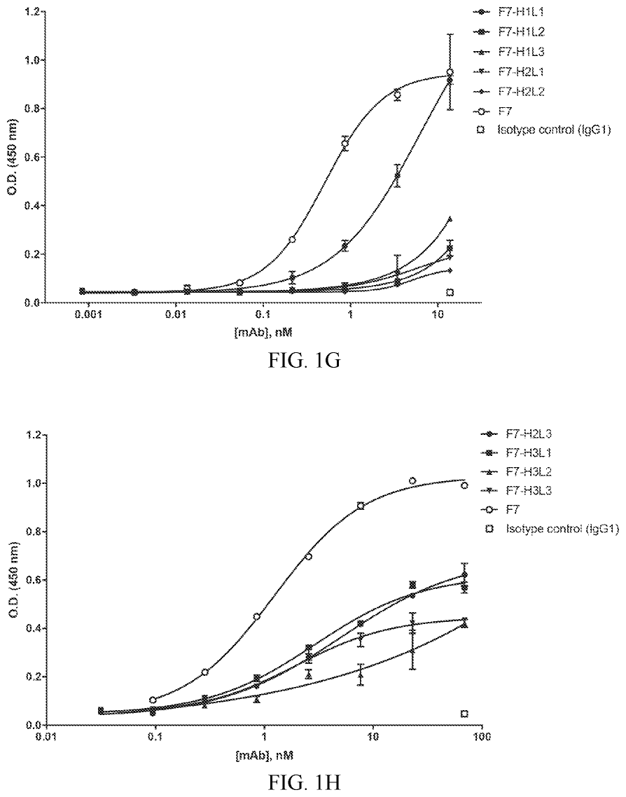

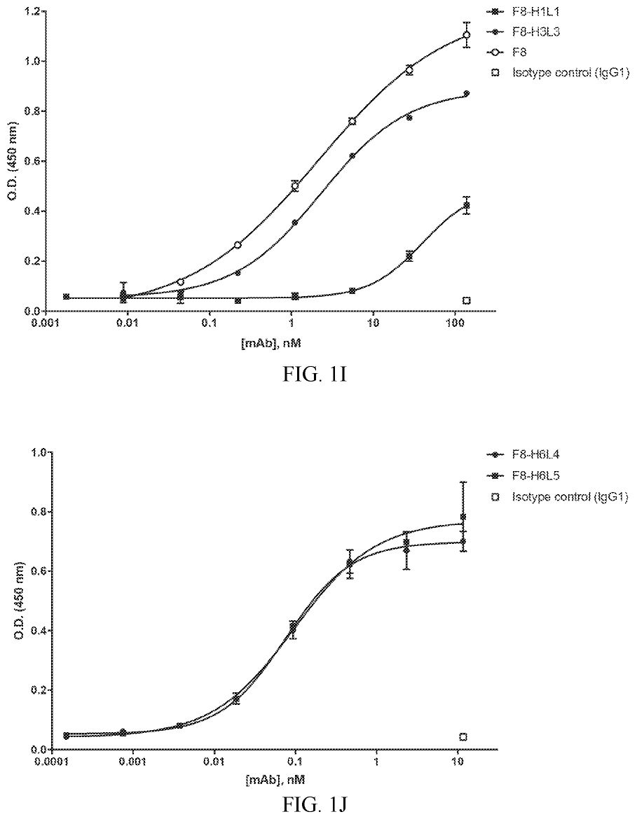

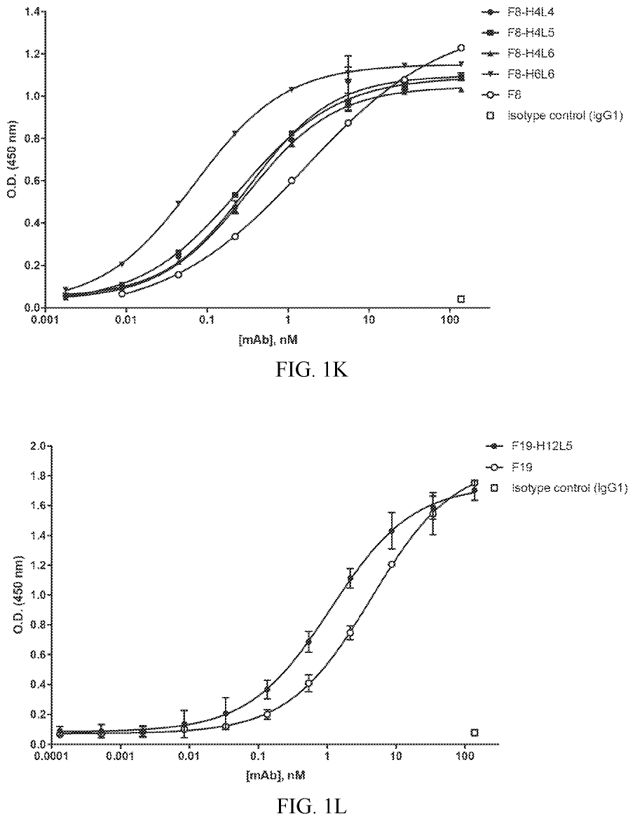

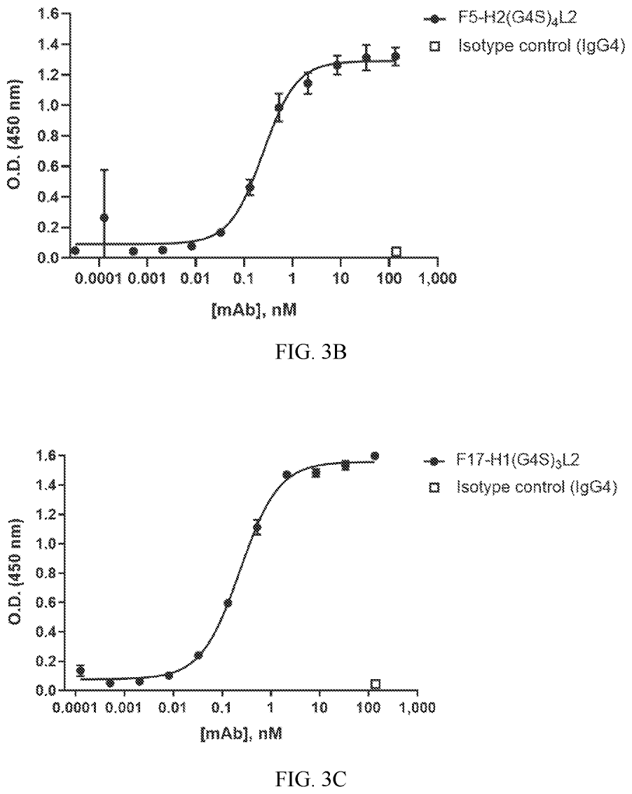

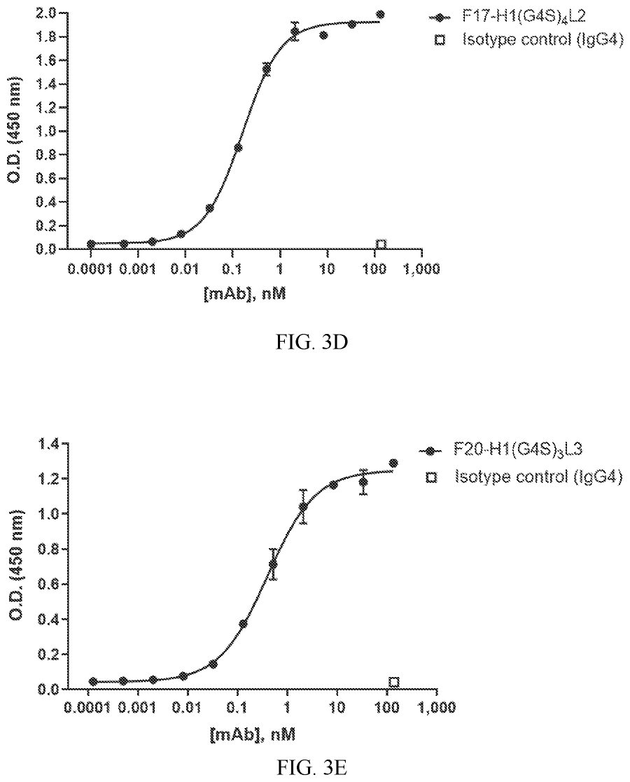

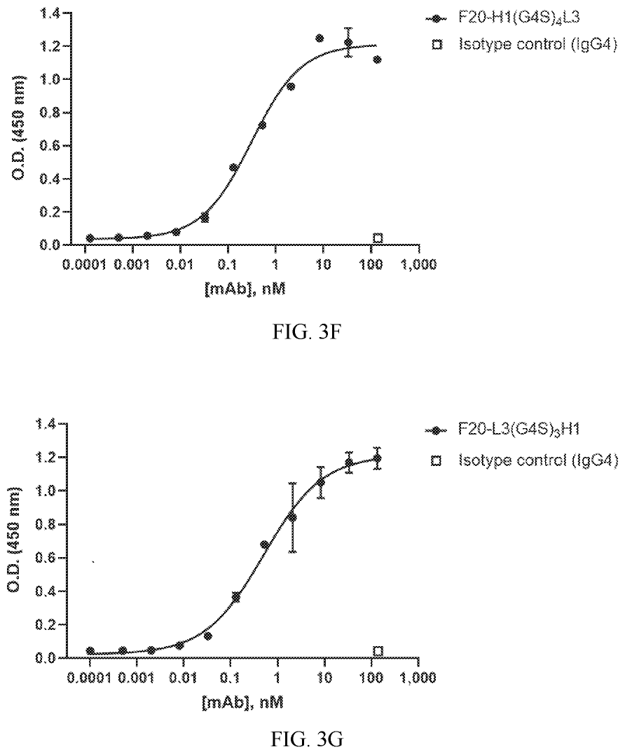

To test the binding of mAbs and scFvs to human FOLR1, mAbs and fusion proteins of scFvs fused to one G₄S linker and human IgG4 Fc (with the order of scFv, G₄S linker and Fc from the N-terminus to the C-terminus) were produced by transient expression in either ExpiCHO or Expi293 cells. The method for the ELISA binding analysis of the mAbs or humanized scFvs is described in PCT/US2019/021084, filed on Mar. 7, 2019. The results of the ELISA assay are shown in FIGS. 1A-1L and FIGS. 3A-3G

### Example 5: FACS Analysis of mAbs and Humanized scFvs

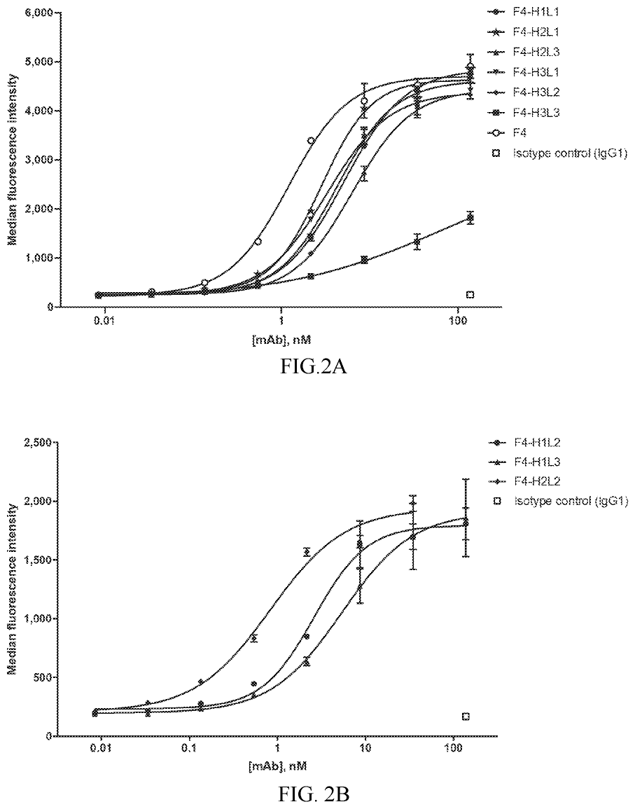

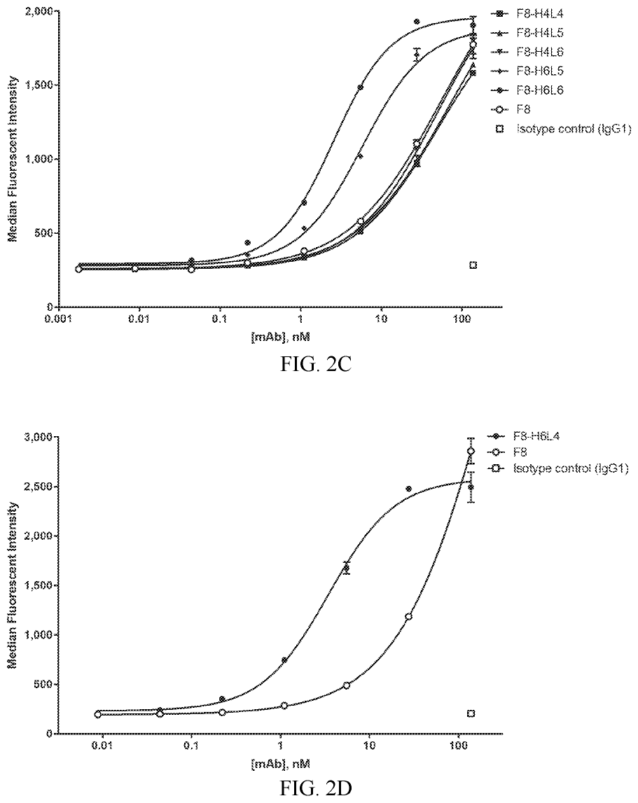

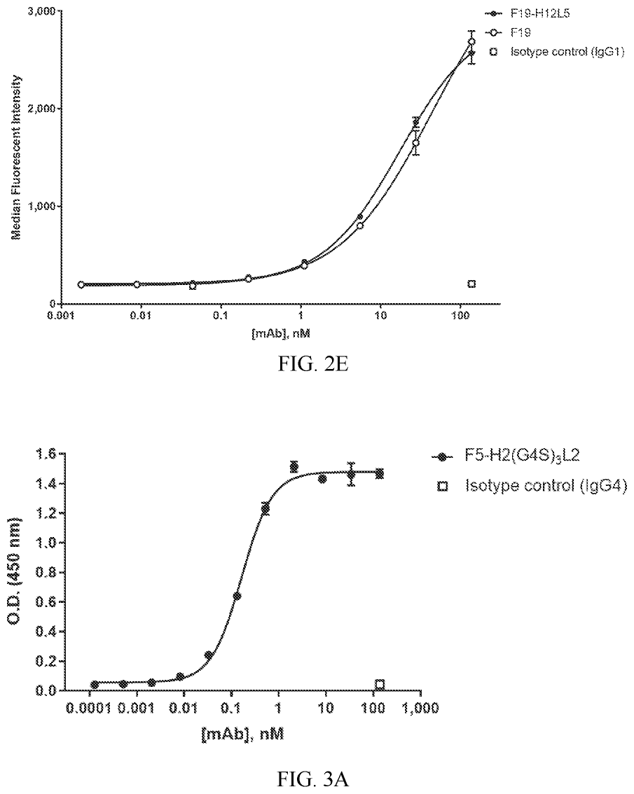

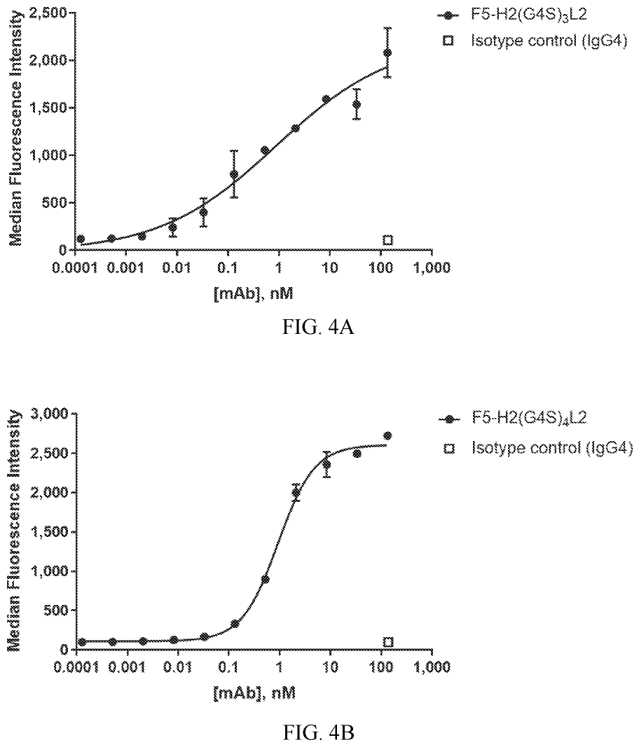

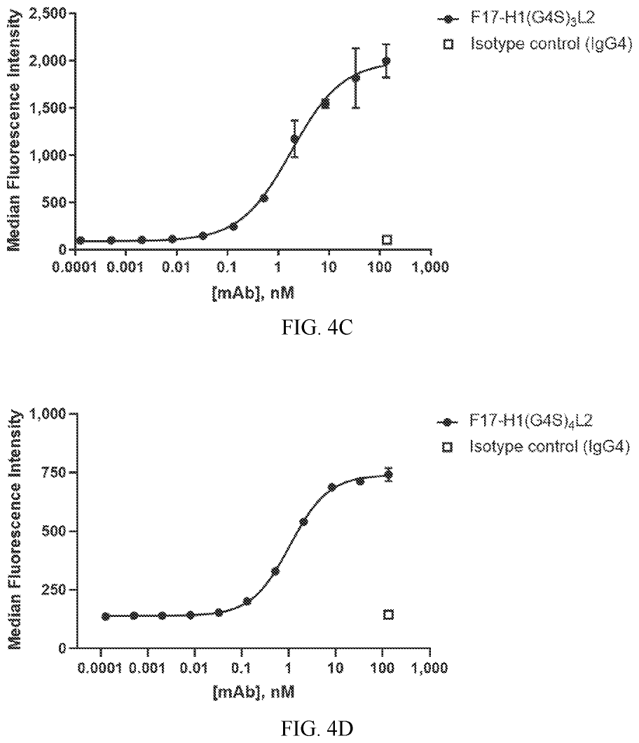

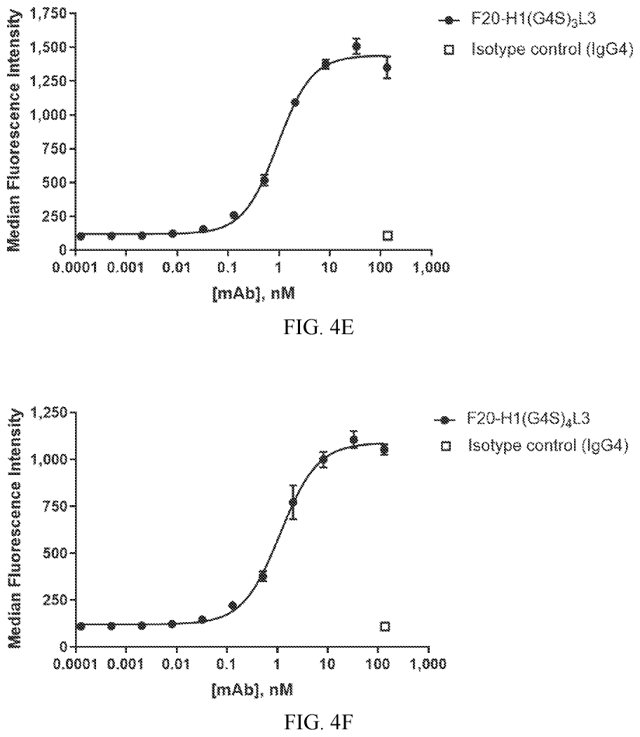

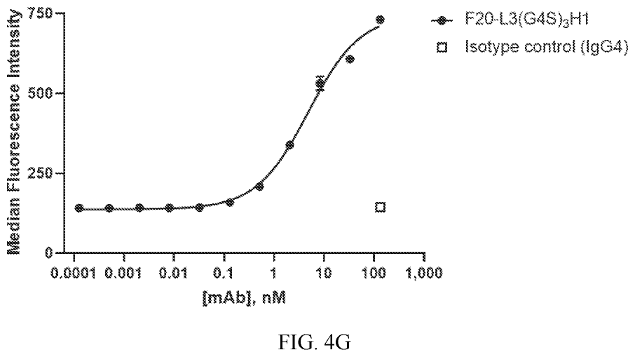

The mAbs and the scFv fusion proteins were also tested for their ability to bind SK-OV-3 cells that express endogenous FOLR1. The method for the FACS analysis of the mAbs or the humanized scFvs is described in PCT/US2019/021084, filed on Mar. 7, 2019, with minor modifications. SK-OV-3 cells were plated at 100,000 cells per well. In each well of the plate, propidium iodide was incubated together with the secondary antibody to label dead cells. The binding results are shown in FIGS. 2A-2E and FIGS. 4A-4G

### Example 6: Construction of Chimeric Antigen Receptor Constructs Comprising Anti-FOLR1 Monoclonal Antibodies or Antigen-Binding Fragments Thereof

To construct a CAR, each mAb is converted into a scFv, using the VH, VL and a (G₄S)n linker, and the scFv is fused to the N-terminus of the hinge and transmembrane domains derived from human CD8a (aa 114-188, Boursier J P et al., J Biol Chem. 1993; 268(3):2013-20). The C-terminal intracellular signaling domain of the CAR is constructed by fusing the intracellular costimulatory domain of CD28 (aa 162-202, Aruffo A and Seed B, Proc Natl Acad Sci USA. 1987; 84(23):8573-7) followed by the activation domain from CD3 zeta chain (aa 52-162, Letourneur F and Klausner R D, Proc Natl Acad Sci USA. 1991; 88(20):8905-9). The DNA sequence encoding the CAR is assembled and cloned into a retroviral vector to generate the CAR construct using standard molecular biology cloning techniques.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated polynucleotide comprising a nucleic acid sequence encoding a chimeric antigen receptor (CAR), wherein the CAR comprises:
(a) an extracellular domain comprising at least one antigen binding domain that specifically binds folate receptor 1 (FOLR1);
(b) a hinge region;
(c) a transmembrane region; and
(d) an intracellular signaling domain.

2. The isolated polynucleotide of claim 1, wherein the antigen binding domain comprises a heavy chain complementarity determining region 1 (HCDR1), HCDR2, HCDR3, a light chain complementarity determining region 1 (LCDR1), LCDR2, and LCDR3, having the polypeptide sequences of:
(1) SEQ ID NOs: 17, 18, 19, 41, 42 and 43, respectively, or SEQ ID NOs: 65, 66, 67, 89, 90 and 91, respectively;
(2) SEQ ID NOs: 20, 21, 22, 44, 45 and 46, respectively, or SEQ ID NOs: 68, 69, 70, 92, 93 and 94, respectively;
(3) SEQ ID NOs: 23, 24, 25, 47, 48 and 49, respectively, or SEQ ID NOs: 71, 72, 73, 95, 96 and 97, respectively;
(4) SEQ ID NOs: 26, 27, 28, 50, 51 and 52, respectively, or SEQ ID NOs: 74, 75, 76, 98, 99 and 100, respectively;
(5) SEQ ID NOs: 29, 30, 31, 53, 54 and 55, respectively, or SEQ ID NOs: 77, 78, 79, 101, 102 and 103, respectively;
(6) SEQ ID NOs: 32, 33, 34, 56, 57 and 58, respectively, or SEQ ID NOs: 80, 81, 82, 104, 105 and 106, respectively;
(7) SEQ ID NOs: 35, 36, 37, 59, 60 and 61, respectively, or SEQ ID NOs: 83, 84, 85, 107, 108 and 109, respectively; or
(8) SEQ ID NOs: 38, 39, 40, 62, 63 and 64, respectively, or SEQ ID NOs: 86, 87, 88, 110, 111 and 112, respectively.

3. (canceled)

4. The isolated polynucleotide of claim 1, wherein the antigen binding domain comprises a heavy chain variable region having a polypeptide sequence at least 95% identical to SEQ ID NO: 1, 3, 5, 7, 9, 11, 13, 15, 113, 114, 115, 116, 119, 120, 124, 125, 128, 129, 130, 136, 137, 138, 142, 143, 144, 148, 149, 150, 154, 155, 156, or 171-184, or a light chain variable region having a polypeptide sequence at least 95% identical to SEQ ID NO: 2, 4, 6, 8, 10, 12, 14, Of 16, 117, 118, 121, 122, 123, 126, 127, 131, 132, 133, 134, 139, 140, 141, 145, 146, 147, 151, 152, 153, 157, 158, or 185-198.

5. The isolated polynucleotide of claim 1, wherein the antigen binding domain comprises:
(1) a heavy chain variable region having the polypeptide sequence of SEQ ID NO:1, and a light chain variable region having the polypeptide sequence of SEQ ID NO:2;
(2) a heavy chain variable region having the polypeptide sequence of SEQ ID NO:3, and a light chain variable region having the polypeptide sequence of SEQ ID NO:4;
(3) a heavy chain variable region having the polypeptide sequence of SEQ ID NO:5, and a light chain variable region having the polypeptide sequence of SEQ ID NO:6;
(4) a heavy chain variable region having the polypeptide sequence of SEQ ID NO:7, and a light chain variable region having the polypeptide sequence of SEQ ID NO:8;
(5) a heavy chain variable region having the polypeptide sequence of SEQ ID NO:9, and a light chain variable region having the polypeptide sequence of SEQ ID NO:10;
(6) a heavy chain variable region having the polypeptide sequence of SEQ ID NO:11, and a light chain variable region having the polypeptide sequence of SEQ ID NO:12;
(7) a heavy chain variable region having the polypeptide sequence of SEQ ID NO:13, and a light chain variable region having the polypeptide sequence of SEQ ID NO:14;
(8) a heavy chain variable region having the polypeptide sequence of SEQ ID NO:15, and a light chain variable region having the polypeptide sequence of SEQ ID NO:16;
(9) a heavy chain variable region having the polypeptide sequence of SEQ ID NO:113, and a light chain variable region having the polypeptide sequence of SEQ ID NO:117;
(10) a heavy chain variable region having the polypeptide sequence of SEQ ID NO:113, and a light chain variable region having the polypeptide sequence of SEQ ID NO:118;
(11) a heavy chain variable region having the polypeptide sequence of SEQ ID NO:114, and a light chain variable region having the polypeptide sequence of SEQ ID NO:117;
(12) a heavy chain variable region having the polypeptide sequence of SEQ ID NO:114, and a light chain variable region having the polypeptide sequence of SEQ ID NO:118;
(13) a heavy chain variable region having the polypeptide sequence of SEQ ID NO:115, and a light chain variable region having the polypeptide sequence of SEQ ID NO:117;
(14) a heavy chain variable region having the polypeptide sequence of SEQ ID NO:115, and a light chain variable region having the polypeptide sequence of SEQ ID NO:118;
(15) a heavy chain variable region having the polypeptide sequence of SEQ ID NO:116, and a light chain variable region having the polypeptide sequence of SEQ ID NO:117;
(16) a heavy chain variable region having the polypeptide sequence of SEQ ID NO:116, and a light chain variable region having the polypeptide sequence of SEQ ID NO:118;
(17) a heavy chain variable region having the polypeptide sequence of SEQ ID NO:119, and a light chain variable region having the polypeptide sequence of SEQ ID NO:121;
(18) a heavy chain variable region having the polypeptide sequence of SEQ ID NO:119, and a light chain variable region having the polypeptide sequence of SEQ ID NO:122;
(19) a heavy chain variable region having the polypeptide sequence of SEQ ID NO:119, and a light chain variable region having the polypeptide sequence of SEQ ID NO:123;
(20) a heavy chain variable region having the polypeptide sequence of SEQ ID NO:120, and a light chain variable region having the polypeptide sequence of SEQ ID NO:121;
(21) a heavy chain variable region having the polypeptide sequence of SEQ ID NO:120, and a light chain variable region having the polypeptide sequence of SEQ ID NO:122;
(22) a heavy chain variable region having the polypeptide sequence of SEQ ID NO:120, and a light chain variable region having the polypeptide sequence of SEQ ID NO:123;
(23) a heavy chain variable region having the polypeptide sequence of SEQ ID NO:124, and a light chain variable region having the polypeptide sequence of SEQ ID NO:126;
(24) a heavy chain variable region having the polypeptide sequence of SEQ ID NO:124, and a light chain variable region having the polypeptide sequence of SEQ ID NO:127;
(25) a heavy chain variable region having the polypeptide sequence of SEQ ID NO:128, and a light chain variable region having the polypeptide sequence of SEQ ID NO:131;
(26) a heavy chain variable region having the polypeptide sequence of SEQ ID NO:128, and a light chain variable region having the polypeptide sequence of SEQ ID NO:132;
(27) a heavy chain variable region having the polypeptide sequence of SEQ ID NO:128, and a light chain variable region having the polypeptide sequence of SEQ ID NO:133;
(28) a heavy chain variable region having the polypeptide sequence of SEQ ID NO:128, and a light chain variable region having the polypeptide sequence of SEQ ID NO:134;
(29) a heavy chain variable region having the polypeptide sequence of SEQ ID NO:129, and a light chain variable region having the polypeptide sequence of SEQ ID NO:131;
(30) a heavy chain variable region having the polypeptide sequence of SEQ ID NO:129, and a light chain variable region having the polypeptide sequence of SEQ ID NO:132;
(31) a heavy chain variable region having the polypeptide sequence of SEQ ID NO:129, and a light chain variable region having the polypeptide sequence of SEQ ID NO:133;
(32) a heavy chain variable region having the polypeptide sequence of SEQ ID NO:129, and a light chain variable region having the polypeptide sequence of SEQ ID NO:134;
(33) a heavy chain variable region having the polypeptide sequence of SEQ ID NO:130, and a light chain variable region having the polypeptide sequence of SEQ ID NO:131;
(34) a heavy chain variable region having the polypeptide sequence of SEQ ID NO:130, and a light chain variable region having the polypeptide sequence of SEQ ID NO:132;
(35) a heavy chain variable region having the polypeptide sequence of SEQ ID NO:130, and a light chain variable region having the polypeptide sequence of SEQ ID NO:133;
(36) a heavy chain variable region having the polypeptide sequence of SEQ ID NO:130, and a light chain variable region having the polypeptide sequence of SEQ ID NO:134;
(37) a heavy chain variable region having the polypeptide sequence of SEQ ID NO:136, and a light chain variable region having the polypeptide sequence of SEQ ID NO:139;
(38) a heavy chain variable region having the polypeptide sequence of SEQ ID NO:136, and a light chain variable region having the polypeptide sequence of SEQ ID NO:140;
(39) a heavy chain variable region having the polypeptide sequence of SEQ ID NO:136, and a light chain variable region having the polypeptide sequence of SEQ ID NO:141;
(40) a heavy chain variable region having the polypeptide sequence of SEQ ID NO:137, and a light chain variable region having the polypeptide sequence of SEQ ID NO:139;
(41) a heavy chain variable region having the polypeptide sequence of SEQ ID NO:137, and a light chain variable region having the polypeptide sequence of SEQ ID NO:140;
(42) a heavy chain variable region having the polypeptide sequence of SEQ ID NO:137, and a light chain variable region having the polypeptide sequence of SEQ ID NO:141;
(43) a heavy chain variable region having the polypeptide sequence of SEQ ID NO:138, and a light chain variable region having the polypeptide sequence of SEQ ID NO:139;
(44) a heavy chain variable region having the polypeptide sequence of SEQ ID NO:138, and a light chain variable region having the polypeptide sequence of SEQ ID NO:140;
(45) a heavy chain variable region having the polypeptide sequence of SEQ ID NO:138, and a light chain variable region having the polypeptide sequence of SEQ ID NO:141;
(46) a heavy chain variable region having the polypeptide sequence of SEQ ID NO:142, and a light chain variable region having the polypeptide sequence of SEQ ID NO:145;
(47) a heavy chain variable region having the polypeptide sequence of SEQ ID NO:142, and a light chain variable region having the polypeptide sequence of SEQ ID NO:146;
(48) a heavy chain variable region having the polypeptide sequence of SEQ ID NO:142, and a light chain variable region having the polypeptide sequence of SEQ ID NO:147;
(49) a heavy chain variable region having the polypeptide sequence of SEQ ID NO:143, and a light chain variable region having the polypeptide sequence of SEQ ID NO:145;
(50) a heavy chain variable region having the polypeptide sequence of SEQ ID NO:143, and a light chain variable region having the polypeptide sequence of SEQ ID NO:146;
(51) a heavy chain variable region having the polypeptide sequence of SEQ ID NO:143, and a light chain variable region having the polypeptide sequence of SEQ ID NO:147;
(52) a heavy chain variable region having the polypeptide sequence of SEQ ID NO:144, and a light chain variable region having the polypeptide sequence of SEQ ID NO:145;
(53) a heavy chain variable region having the polypeptide sequence of SEQ ID NO:144, and a light chain variable region having the polypeptide sequence of SEQ ID NO:146;
(54) a heavy chain variable region having the polypeptide sequence of SEQ ID NO:144, and a light chain variable region having the polypeptide sequence of SEQ ID NO:147;
(55) a heavy chain variable region having the polypeptide sequence of SEQ ID NO:148, and a light chain variable region having the polypeptide sequence of SEQ ID NO:151;
(56) a heavy chain variable region having the polypeptide sequence of SEQ ID NO:150, and a light chain variable region having the polypeptide sequence of SEQ ID NO:153;
(57) a heavy chain variable region having the polypeptide sequence of SEQ ID NO:171, and a light chain variable region having the polypeptide sequence of SEQ ID NO:185;
(58) a heavy chain variable region having the polypeptide sequence of SEQ ID NO:171, and a light chain variable region having the polypeptide sequence of SEQ ID NO:186;
(59) a heavy chain variable region having the polypeptide sequence of SEQ ID NO:171, and a light chain variable region having the polypeptide sequence of SEQ ID NO:187;
(60) a heavy chain variable region having the polypeptide sequence of SEQ ID NO:173, and a light chain variable region having the polypeptide sequence of SEQ ID NO:185;
(61) a heavy chain variable region having the polypeptide sequence of SEQ ID NO:173, and a light chain variable region having the polypeptide sequence of SEQ ID NO:186;
(62) a heavy chain variable region having the polypeptide sequence of SEQ ID NO:173, and a light chain variable region having the polypeptide sequence of SEQ ID NO:187;
(63) a heavy chain variable region having the polypeptide sequence of SEQ ID NO:182, and a light chain variable region having the polypeptide sequence of SEQ ID NO:190;
(64) a heavy chain variable region having the polypeptide sequence of SEQ ID NO:183, and a light chain variable region having the polypeptide sequence of SEQ ID NO:195;
(65) a heavy chain variable region having the polypeptide sequence of SEQ ID NO:183, and a light chain variable region having the polypeptide sequence of SEQ ID NO:196;
(66) a heavy chain variable region having the polypeptide sequence of SEQ ID NO:183, and a light chain variable region having the polypeptide sequence of SEQ ID NO:197;
(67) a heavy chain variable region having the polypeptide sequence of SEQ ID NO:183, and a light chain variable region having the polypeptide sequence of SEQ ID NO:198;
(68) a heavy chain variable region having the polypeptide sequence of SEQ ID NO:184, and a light chain variable region having the polypeptide sequence of SEQ ID NO:195;
(69) a heavy chain variable region having the polypeptide sequence of SEQ ID NO:184, and a light chain variable region having the polypeptide sequence of SEQ ID NO:196;
(70) a heavy chain variable region having the polypeptide sequence of SEQ ID NO:184, and a light chain variable region having the polypeptide sequence of SEQ ID NO:197; or
(71) a heavy chain variable region having the polypeptide sequence of SEQ ID NO:184, and a light chain variable region having the polypeptide sequence of SEQ ID NO:198.

6-7. (canceled)

8. The isolated polynucleotide of claim 1, wherein the antigen binding domain is a single chain variable fragment (scFv).

9. The isolated polynucleotide of claim 8, wherein the single chain variable fragment (scFv) is humanized.

10. The isolated polynucleotide of claim 8 or 9, wherein the single chain variable fragment (scFv) comprises a polypeptide sequence at least 95% identical to any one of SEQ ID NOs: 159-170.

11. The isolated polynucleotide of claim 1, wherein the chimeric antigen receptor (CAR) comprises one or more antigen binding domains, and/or wherein the intracellular signaling domain comprises one or more costimulatory domains and one or more activating domains.

12. (canceled)

13. A chimeric antigen receptor (CAR) encoded by the isolated polynucleotide of claim 1.

14. A vector comprising the isolated polynucleotide of claim 1.

15. A host cell comprising the vector of claim 14.

16. The host cell of claim 15, wherein the host cell is a T cell or a NK cell.

17. (canceled)

18. A method of making a host cell expressing a chimeric antigen receptor (CAR), the method comprising transducing a T cell or a NK cell with the vector of claim 14.

19. A method of producing a chimeric antigen receptor (CAR)-T cell or a chimeric antigen receptor (CAR)-NK cell, the method comprising culturing T cells or NK cells comprising the isolated polynucleotide comprising a nucleic acid encoding a chimeric antigen receptor (CAR) of claim 1 under conditions to produce the CAR-T cell or CAR-NK cell and recovering the CAR-T cell or CAR-NK cell.

20-21. (canceled)

22. A method of generating a cell comprising a chimeric antigen receptor (CAR), the method comprising contacting a cell with the isolated polynucleotide comprising a nucleic acid encoding a chimeric antigen receptor (CAR) of claim 1, wherein the isolated polynucleotide is an in vitro transcribed RNA or synthetic RNA.

23. A method of treating cancer in a subject in need thereof, the method comprising administering to the subject in need thereof the host cell of claim 15.

24. The method of claim 23, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CIVIL), a multiple myeloma (MM), an acute myeloid leukemia (AML), and other liquid tumors.

25. The method of claim 23, further comprising administering to the subject in need thereof an agent that increases the efficacy of a cell expressing a CAR, an agent that ameliorates one or more side effects associated with administration of a cell expressing a CAR, or an agent that treats the disease associated with FOLR1.

26-27. (canceled)

28. A humanized anti-FOLR1 monoclonal antibody or antigen-binding fragment thereof, wherein the antibody or antigen-binding fragment thereof comprises a heavy chain variable region having a polypeptide sequence at least 95% identical to any one of SEQ ID NO: 113, 114, 115, 116, 119, 120, 124, 125, 128, 129, 130, 136, 137, 138, 142, 143, 144, 148, 149, 150, 154, 155, 156 or 171-184, or a light chain variable region having a polypeptide sequence at least 95% identical to SEQ ID NO: 117, 118, 121, 122, 123, 126, 127, 131, 132, 133, 134, 139, 140, 141, 145, 146, 147, 151, 152, 153, 157, 158 or 185-198.

29. The humanized anti-FOLR1 monoclonal antibody or antigen-binding fragment thereof of claim 28, wherein the antibody or antigen-binding fragment thereof comprises:
(1) a heavy chain variable region having the polypeptide sequence of SEQ ID NO:113, and a light chain variable region having the polypeptide sequence of SEQ ID NO:117;
(2) a heavy chain variable region having the polypeptide sequence of SEQ ID NO:113, and a light chain variable region having the polypeptide sequence of SEQ ID NO:118;
(3) a heavy chain variable region having the polypeptide sequence of SEQ ID NO:114, and a light chain variable region having the polypeptide sequence of SEQ ID NO:117;
(4) a heavy chain variable region having the polypeptide sequence of SEQ ID NO:114, and a light chain variable region having the polypeptide sequence of SEQ ID NO:118;
(5) a heavy chain variable region having the polypeptide sequence of SEQ ID NO:115, and a light chain variable region having the polypeptide sequence of SEQ ID NO:117;
(6) a heavy chain variable region having the polypeptide sequence of SEQ ID NO:115, and a light chain variable region having the polypeptide sequence of SEQ ID NO:118;
(7) a heavy chain variable region having the polypeptide sequence of SEQ ID NO:116, and a light chain variable region having the polypeptide sequence of SEQ ID NO:117;
(8) a heavy chain variable region having the polypeptide sequence of SEQ ID NO:116, and a light chain variable region having the polypeptide sequence of SEQ ID NO:118;
(9) a heavy chain variable region having the polypeptide sequence of SEQ ID NO:119, and a light chain variable region having the polypeptide sequence of SEQ ID NO:121;
(10) a heavy chain variable region having the polypeptide sequence of SEQ ID NO:119, and a light chain variable region having the polypeptide sequence of SEQ ID NO:122;
(11) a heavy chain variable region having the polypeptide sequence of SEQ ID NO:119, and a light chain variable region having the polypeptide sequence of SEQ ID NO:123;
(12) a heavy chain variable region having the polypeptide sequence of SEQ ID NO:120, and a light chain variable region having the polypeptide sequence of SEQ ID NO:121;
(13) a heavy chain variable region having the polypeptide sequence of SEQ ID NO:120, and a light chain variable region having the polypeptide sequence of SEQ ID NO:122;
(14) a heavy chain variable region having the polypeptide sequence of SEQ ID NO:120, and a light chain variable region having the polypeptide sequence of SEQ ID NO:123;
(15) a heavy chain variable region having the polypeptide sequence of SEQ ID NO:124, and a light chain variable region having the polypeptide sequence of SEQ ID NO:126;
(16) a heavy chain variable region having the polypeptide sequence of SEQ ID NO:124, and a light chain variable region having the polypeptide sequence of SEQ ID NO:127;
(17) a heavy chain variable region having the polypeptide sequence of SEQ ID NO:128, and a light chain variable region having the polypeptide sequence of SEQ ID NO:131;
(18) a heavy chain variable region having the polypeptide sequence of SEQ ID NO:128, and a light chain variable region having the polypeptide sequence of SEQ ID NO:132;
(19) a heavy chain variable region having the polypeptide sequence of SEQ ID NO:128, and a light chain variable region having the polypeptide sequence of SEQ ID NO:133;
(20) a heavy chain variable region having the polypeptide sequence of SEQ ID NO:128, and a light chain variable region having the polypeptide sequence of SEQ ID NO:134;
(21) a heavy chain variable region having the polypeptide sequence of SEQ ID NO:129, and a light chain variable region having the polypeptide sequence of SEQ ID NO:131;
(22) a heavy chain variable region having the polypeptide sequence of SEQ ID NO:129, and a light chain variable region having the polypeptide sequence of SEQ ID NO:132;
(23) a heavy chain variable region having the polypeptide sequence of SEQ ID NO:129, and a light chain variable region having the polypeptide sequence of SEQ ID NO:133;
(24) a heavy chain variable region having the polypeptide sequence of SEQ ID NO:129, and a light chain variable region having the polypeptide sequence of SEQ ID NO:134;
(25) a heavy chain variable region having the polypeptide sequence of SEQ ID NO:130, and a light chain variable region having the polypeptide sequence of SEQ ID NO:131;
(26) a heavy chain variable region having the polypeptide sequence of SEQ ID NO:130, and a light chain variable region having the polypeptide sequence of SEQ ID NO:132;
(27) a heavy chain variable region having the polypeptide sequence of SEQ ID NO:130, and a light chain variable region having the polypeptide sequence of SEQ ID NO:133;
(28) a heavy chain variable region having the polypeptide sequence of SEQ ID NO:130, and a light chain variable region having the polypeptide sequence of SEQ ID NO:134;
(29) a heavy chain variable region having the polypeptide sequence of SEQ ID NO:136, and a light chain variable region having the polypeptide sequence of SEQ ID NO:139;
(30) a heavy chain variable region having the polypeptide sequence of SEQ ID NO:136, and a light chain variable region having the polypeptide sequence of SEQ ID NO:140;
(31) a heavy chain variable region having the polypeptide sequence of SEQ ID NO:136, and a light chain variable region having the polypeptide sequence of SEQ ID NO:141;
(32) a heavy chain variable region having the polypeptide sequence of SEQ ID NO:137, and a light chain variable region having the polypeptide sequence of SEQ ID NO:139;
(33) a heavy chain variable region having the polypeptide sequence of SEQ ID NO:137, and a light chain variable region having the polypeptide sequence of SEQ ID NO:140;
(34) a heavy chain variable region having the polypeptide sequence of SEQ ID NO:137, and a light chain variable region having the polypeptide sequence of SEQ ID NO:141;
(35) a heavy chain variable region having the polypeptide sequence of SEQ ID NO:138, and a light chain variable region having the polypeptide sequence of SEQ ID NO:139;
(36) a heavy chain variable region having the polypeptide sequence of SEQ ID NO:138, and a light chain variable region having the polypeptide sequence of SEQ ID NO:140;
(37) a heavy chain variable region having the polypeptide sequence of SEQ ID NO:138, and a light chain variable region having the polypeptide sequence of SEQ ID NO:141;
(38) a heavy chain variable region having the polypeptide sequence of SEQ ID NO:142, and a light chain variable region having the polypeptide sequence of SEQ ID NO:145;
(39) a heavy chain variable region having the polypeptide sequence of SEQ ID NO:142, and a light chain variable region having the polypeptide sequence of SEQ ID NO:146;
(40) a heavy chain variable region having the polypeptide sequence of SEQ ID NO:142, and a light chain variable region having the polypeptide sequence of SEQ ID NO:147;
(41) a heavy chain variable region having the polypeptide sequence of SEQ ID NO:143, and a light chain variable region having the polypeptide sequence of SEQ ID NO:145;
(42) a heavy chain variable region having the polypeptide sequence of SEQ ID NO:143, and a light chain variable region having the polypeptide sequence of SEQ ID NO:146;
(43) a heavy chain variable region having the polypeptide sequence of SEQ ID NO:143, and a light chain variable region having the polypeptide sequence of SEQ ID NO:147;
(44) a heavy chain variable region having the polypeptide sequence of SEQ ID NO:144, and a light chain variable region having the polypeptide sequence of SEQ ID NO:145;
(45) a heavy chain variable region having the polypeptide sequence of SEQ ID NO:144, and a light chain variable region having the polypeptide sequence of SEQ ID NO:146;
(46) a heavy chain variable region having the polypeptide sequence of SEQ ID NO:144, and a light chain variable region having the polypeptide sequence of SEQ ID NO:147;
(47) a heavy chain variable region having the polypeptide sequence of SEQ ID NO:148, and a light chain variable region having the polypeptide sequence of SEQ ID NO:151;
(48) a heavy chain variable region having the polypeptide sequence of SEQ ID NO:150, and a light chain variable region having the polypeptide sequence of SEQ ID NO:153;
(49) a heavy chain variable region having the polypeptide sequence of SEQ ID NO:171, and a light chain variable region having the polypeptide sequence of SEQ ID NO:185;
(50) a heavy chain variable region having the polypeptide sequence of SEQ ID NO:171, and a light chain variable region having the polypeptide sequence of SEQ ID NO:186;
(51) a heavy chain variable region having the polypeptide sequence of SEQ ID NO:171, and a light chain variable region having the polypeptide sequence of SEQ ID NO:187;
(52) a heavy chain variable region having the polypeptide sequence of SEQ ID NO:173, and a light chain variable region having the polypeptide sequence of SEQ ID NO:185;
(53) a heavy chain variable region having the polypeptide sequence of SEQ ID NO:173, and a light chain variable region having the polypeptide sequence of SEQ ID NO:186;
(54) a heavy chain variable region having the polypeptide sequence of SEQ ID NO:173, and a light chain variable region having the polypeptide sequence of SEQ ID NO:187;
(55) a heavy chain variable region having the polypeptide sequence of SEQ ID NO:182, and a light chain variable region having the polypeptide sequence of SEQ ID NO:190;
(56) a heavy chain variable region having the polypeptide sequence of SEQ ID NO:183, and a light chain variable region having the polypeptide sequence of SEQ ID NO:195;
(57) a heavy chain variable region having the polypeptide sequence of SEQ ID NO:183, and a light chain variable region having the polypeptide sequence of SEQ ID NO:196;
(58) a heavy chain variable region having the polypeptide sequence of SEQ ID NO:183, and a light chain variable region having the polypeptide sequence of SEQ ID NO:197;
(59) a heavy chain variable region having the polypeptide sequence of SEQ ID NO:183, and a light chain variable region having the polypeptide sequence of SEQ ID NO:198;
(60) a heavy chain variable region having the polypeptide sequence of SEQ ID NO:184, and a light chain variable region having the polypeptide sequence of SEQ ID NO:195;
(61) a heavy chain variable region having the polypeptide sequence of SEQ ID NO:184, and a light chain variable region having the polypeptide sequence of SEQ ID NO:196;
(62) a heavy chain variable region having the polypeptide sequence of SEQ ID NO:184, and a light chain variable region having the polypeptide sequence of SEQ ID NO:197; or
(63) a heavy chain variable region having the polypeptide sequence of SEQ ID NO:184, and a light chain variable region having the polypeptide sequence of SEQ ID NO:198.

30. The humanized anti-FOLR1 monoclonal antibody or antigen-binding fragment thereof of claim 28, wherein the monoclonal antibody or antigen-binding fragment thereof is capable of binding FOLR1, inducing effector-mediated tumor cell lysis, mediating the recruitment of conjugated drugs, and/or forming a bispecific antibody with another monoclonal antibody or antigen-binding fragment with a cancer-killing effect.

31. An isolated nucleic acid encoding the anti-FOLR1 monoclonal antibody or antigen-binding fragment thereof of claim 1.

32. A vector comprising the isolated nucleic acid of claim 31.

33. A host cell comprising the vector of claim 32.

34. A pharmaceutical composition, comprising the anti-FOLR1 monoclonal antibody or antigen-binding fragment thereof of claim 28 and a pharmaceutically acceptable carrier.

35. A method of targeting FOLR1 on a cancer cell surface or a method of treating cancer in a subject in need thereof, the method comprising administering to the subject in need thereof the pharmaceutical composition of claim 34.

36. (canceled)

37. The method of claim 35, wherein the cancer is selected from a lung cancer, a gastric cancer, a colon cancer, a hepatocellular carcinoma, a renal cell carcinoma, a bladder urothelial carcinoma, a metastatic melanoma, a breast cancer, an ovarian cancer, a cervical cancer, a head and neck cancer, a pancreatic cancer, a glioma, a glioblastoma, and other solid tumors, and a non-Hodgkin's lymphoma (NHL), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors.

38. A method of producing the anti-FOLR1 monoclonal antibody or antigen-binding fragment thereof of claim 28, comprising culturing a cell comprising a nucleic acid encoding the monoclonal antibody or antigen-binding fragment under conditions to produce the monoclonal antibody or antigen-binding fragment, and recovering the antibody or antigen-binding fragment from the cell or culture.

39. A method of producing the pharmaceutical composition of claim 34, comprising combining the monoclonal antibody or antigen-binding fragment thereof with a pharmaceutically acceptable carrier to obtain the pharmaceutical composition.
